SEQUENCE LISTING (4 sequences)

SEQ ID NO: 1 - DNA, 321 bp, Homo sapiens
gggtccctgagactctcctgtgcagcctctggattcacctttagcagctatgccatgagctgggtccgccaggctccagggaaggggctggagtgggtctcagctattagtggtagtggtggtagtacatactacgcagactccgtgaagggccggttcaccatctccagagacaattccaagaacacgctgtatctgcaaatgaacagcctgagagccgaggacacggccgtatattactgtgcgaaaggtggggccgaaggctggtacgagtactactactactacggtatggacgtctggggccaagggaccctggtc

SEQ ID NO: 2 - DNA, 348 bp, Homo sapiens
cagtctgccctgactcagcctgcttccctctctgcatctcctggagcatcaggcagtctcacctgcaccttgcgcagtggcatcaatgttggtacctacaggatatactggtaccagcagaagccagggagtcctccccagtatctcctgaggtacaaatcagactcagataagcagaagggctctggagtccccagccgcttctctggatccaaagatgcttcggccaatgcagggattttactcatctctgggctccagtctgaggatgaggctgactattactgtatgatttggcacagcagcgcttgggtgttcggcggagggaccaagctgaccgtcctaggt

SEQ ID NO: 3 - protein, 107 aa, Homo sapiens
GSLRLSCAASGFTFSSYAMSWVRQAPGKGLEWVSAISGSGGSTYYADSVKGRFTISRDNSKNTLYLQMNSLRAEDTAVYYCAKGGAEGWYEYYYYYGMDVWGQGTLV

SEQ ID NO: 4 - protein, 116 aa, Homo sapiens
QSALTQPASLSASPGASASLTCTLRSGINVGTYRIYWYQQKPGSPPQYLLRYKSDSDKQKGSGVPSRFSGSKDASANAGILLISGLQSEDEADYYCMIWHSSAWVFGGGTKLTVLG

(19) United States
(12) Patent Application Publication
(10) Pub. No.: US 2009/0202570 A1
(43) Pub. Date: Aug. 13, 2009

(54) PM-2 ANTIBODIES, FUNCTIONAL FRAGMENTS AND METHODS FOR TREATING METASTASIS

(75) Inventors: Hans-Konrad Mueller-Hermelink, Wurzburg (DE); Heinz Vollmers, Wurzburg (DE); Bertram Illert, Wurzburg (DE)

Correspondence Address: PILLSBURY WINTHROP SHAW PITTMAN LLP, ATTENTION: DOCKETING DEPARTMENT, P.O BOX 10500, McLean, VA, 22102, US

(73) Assignee: PATRYS LIMITED, Melbourne (AU)

(21) Appl. No.: 12/339,737

(22) Filed: Dec. 19, 2008

Related U.S. Application Data
(60) Provisional application No. 61/016,219, filed on Dec. 21, 2007.

(51) Int. Cl. A61K 39/395; A61P 35/00

(52) U.S. Cl. 424/174.1

(57) ABSTRACT

The invention provides PM-2 antibodies, functional fragments, modified and variant forms, nucleic acid and other compositions. Antibodies, functional fragments, modified and variant forms, nucleic acid and other compositions are useful in treatment and diagnostic methods. One method includes treating metastasis of a neoplasia, tumor or cancer in a subject in need of treatment by administering to the subject an amount of a PM-2 antibody or functional fragment thereof effective to treat metastasis of the neoplasia, tumor or cancer in the subject.

### RELATED APPLICATIONS

This application claims priority to application Ser. No. 61/016,219, filed Dec. 21, 2007, which is expressly incorporated by reference in its entirety.

### FIELD OF THE INVENTION

The invention relates to an antibody, known as PM-2 (WO 2004/005351). The antibody denoted PM-2 is an IgM and binds to different types of neoplasia, cancer, tumor and metastasis. PM-2 inhibits growth of various types of cancer cells and stimulates or induces apoptosis of various types of cancer cells. PM-2 also reduces formation or establishment of metastases at one or more sites arising from a primary neoplasia, tumor or cancer, or growth or proliferation of a metastasis that has formed or been established at one or more other sites.

### INTRODUCTION

Disseminated tumor cells (DTC) in various body compartments potentially contribute to cancer progression and relapse. DTC may be related to the eventual development of metastatic disease at sites peripheral to the primary cancer. Consequently, a reduction of DTC is likely to reduce or inhibit establishment or formation of metastatic tumors, or growth or proliferation of established metastatic tumors. The invention addresses this need and provides related benefits.

### SUMMARY

The invention provides isolated and purified antibodies and functional fragments that compete for binding to a cell, antigen or epitope that PM-2 antibody, produced by a cell line DSMZ Deposit No. DSM ACC 2600, or represented by heavy and light chain sequences set forth as SEQ ID NOs:1 and 2, binds. In one embodiment, an antibody or functional fragment competes with PM-2 antibody, produced by a cell line DSMZ Deposit No. DSM ACC 2600, or represented by heavy and light chain sequences set forth as SEQ ID NOs:1 and 2, for binding to an neoplastic, tumor or cancer or a metastatic cell.

In particular aspects, an antibody or functional fragment competes with PM-2 antibody, produced by a cell line DSMZ Deposit No. DSM ACC 2600, or represented by heavy and light chain sequences set forth as SEQ ID NOs:1 and 2, for binding to an antigen on one or more of a stomach adenocarcinoma, colorectal adenocarcinoma, squamous cell lung carcinoma, lung adenocarcinoma, squamous cell carcinoma of the esophagus, adenocarcinoma of the pancreas, urothel carcinoma of the urinary bladder, renal cell carcinoma of the kidney, adenocarcinoma of the prostate, ductal carcinoma of the breast, lobular carcinoma of the breast, adenocarcinoma of the ovary, adenocarcinoma of the adrenal gland, adenocarcinoma of the endometrium or an adenocarcinoma of a uterus cell. In another embodiment, an antibody or functional fragment competes with PM-2 antibody, produced by a cell line DSMZ Deposit No. DSM ACC 2600, or represented by heavy and light chain sequences set forth as SEQ ID NOs:1 and 2, for binding to a stomach adenocarcinoma, colorectal adenocarcinoma, squamous cell lung carcinoma, lung adenocarcinoma, squamous cell carcinoma of the esophagus, adenocarcinoma of the pancreas, urothel carcinoma of the urinary bladder, renal cell carcinoma of the kidney, adenocarcinoma of the prostate, ductal carcinoma of the breast, lobular carcinoma of the breast, adenocarcinoma of the ovary, adenocarcinoma of the adrenal gland, adenocarcinoma of the endometrium or adenocarcinoma of the uterus cell. In an additional embodiment, an antibody or functional fragment competes with PM-2 antibody, produced by a cell line DSMZ Deposit No. DSM ACC 2600, or represented by heavy and light chain sequences set forth as SEQ ID NOs:1 and 2, for binding to HT-29 (ATCC Accession No. HTB-38; DSMZ Accession No. ACC 299), CACO-2 (ATCC Accession No. HBT-37; DSMZ Accession No. ACC 169), COLO-320 (DSMZ Accession No. ACC 144), COLO-206F (DSMZ Accession No. ACC 21), ASPC-1 (ATCC Accession No. CRL-1682), BXPC-3 (ATCC Accession No. CRL-1687) or A549 (ATCC Accession No. CCL-185) cells. In a further embodiment, an antibody or functional fragment thereof inhibits or reduces proliferation, or stimulates or induces apoptosis, of one or more of a stomach adenocarcinoma, colorectal adenocarcinoma, squamous cell lung carcinoma, lung adenocarcinoma, squamous cell carcinoma of the esophagus, adenocarcinoma of the pancreas, urothel carcinoma of the urinary bladder, renal cell carcinoma of the kidney, adenocarcinoma of the prostate, ductal carcinoma of the breast, lobular carcinoma of the breast, adenocarcinoma of the ovary, adenocarcinoma of the adrenal gland, adenocarcinoma of the endometrium a uterus cell adenocarcinoma, or an HT-29 (ATCC Accession No. HTB-38; DSMZ Accession No. ACC 299), CACO-2 (ATCC Accession No. HBT-37; DSMZ Accession No. ACC 169), COLO-320 (DSMZ Accession No. ACC 144), COLO-206F (DSMZ Accession No. ACC 21), ASPC-1 (ATCC Accession No. CRL-1682), BXPC-3 (ATCC Accession No. CRL-1687) or A549 (ATCC Accession No. CCL-185) cells.

The invention also provides isolated and purified antibodies and functional fragments thereof that bind to cells, antigen or epitope that PM-2 antibody, produced by a cell line DSMZ Deposit No. DSM ACC 2600, or represented by heavy and light chain sequences set forth as SEQ ID NOs:1 and 2, binds. In one embodiment, an antibody or functional fragment binds to an adenocarcinoma cell or a squamous cell carcinoma to which PM-2 antibody, produced by a cell line DSMZ Deposit No. DSM ACC 2600, or represented by heavy and light chain sequences set forth as SEQ ID NOs:1 and 2, binds. In another embodiment, an antibody or functional fragment thereof binds to an epitope or an antigen to which PM-2 antibody, produced by a cell line DSMZ Deposit No. DSM ACC 2600, or represented by heavy and light chain sequences set forth as SEQ ID NOs:1 and 2 binds (e.g., a protein antigen of about 115 kDa expressed on BXPC-3, MKN or CRL cells). In a further embodiment, an antibody or functional fragment thereof competes with PM-2 antibody, produced by a cell line DSMZ Deposit No. DSM ACC 2600, or represented by heavy and light chain sequences set forth as SEQ ID NOs:1 and 2, for binding to HT-29, CACO-2, COLO-320, COLO-206F, ASPC-1, BXPC-3 or A549 cells. In yet another embodiment, an antibody or functional fragment thereof binds to an epitope to which PM-2 antibody, produced by a cell line DSMZ Deposit No. DSM ACC 2600, or represented by heavy and light chain sequences set forth as SEQ ID NOs:1 and 2 binds, wherein the epitope is present on a protein antigen of about 115 kDa expressed on BXPC-3, MKN or CRL cells. In particular aspects, an antibody or functional fragment binds to one or more of a stomach adenocarcinoma, colorectal adenocarcinoma, squamous cell lung carcinoma, lung adenocarcinoma, squamous cell carcinoma of the esophagus, adenocarcinoma of the pancreas, urothel carcinoma of the urinary bladder, renal cell carcinoma of the kidney, adenocarcinoma of the prostate, ductal carcinoma of the breast, lobular carcinoma of the breast, adenocarcinoma of the ovary, adenocarcinoma of the adrenal gland, adenocarcinoma of the endometrium or a uterus cell adenocarcinoma, to which PM-2 antibody, produced by a cell line DSMZ Deposit No. DSM ACC 2600, or represented by heavy and light chain sequences set forth as SEQ ID NOs:1 and 2, binds. In another embodiment, an antibody or functional fragment binds to a stomach adenocarcinoma, colorectal adenocarcinoma, squamous cell lung carcinoma, lung adenocarcinoma, squamous cell carcinoma of the esophagus, adenocarcinoma of the pancreas, urothel carcinoma of the urinary bladder, renal cell carcinoma of the kidney, adenocarcinoma of the prostate, ductal carcinoma of the breast, lobular carcinoma of the breast, adenocarcinoma of the ovary, adenocarcinoma of the adrenal gland, adenocarcinoma of the endometrium or a uterus cell adenocarcinoma to which PM-2 antibody, produced by a cell line DSMZ Deposit No. DSM ACC 2600, or represented by heavy and light chain sequences set forth as SEQ ID NOs:1 and 2, binds. In an additional embodiment, an antibody or functional fragment binds to an HT-29 (ATCC Accession No. HTB-38; DSMZ Accession No. ACC 299), CACO-2 (ATCC Accession No. HBT-37; DSMZ Accession No. ACC 169), COLO-320 (DSMZ Accession No. ACC 144), COLO-206F (DSMZ Accession No. ACC 21), ASPC-1 (ATCC Accession No. CRL-1682), BXPC-3 (ATCC Accession No. CRL-1687) or A549 (ATCC Accession No. CCL-185) cells that PM-2 antibody, produced by a cell line DSMZ Deposit No. DSM ACC 2600, or represented by heavy and light chain sequences set forth as SEQ ID NOs:1 and 2, binds.

The invention further provides isolated and purified antibodies and functional fragments that include a heavy or light chain variable region sequence with about 60% or more identity to a heavy or light chain sequence variable regions of PM-2 antibody, produced by a cell line DSMZ Deposit No. DSM ACC 2600, or represented by heavy and light chain sequences set forth as SEQ ID NOs:1 and 2. In one embodiment, an antibody or subsequence thereof includes a sequence at least 60% or more (e.g., 65%, 70%, 75%, 80%, 85%, 90%, 95%, etc.) identical to a heavy chain variable region sequence set forth as SEQ ID NO:1, or a sequence at least 60% or more (e.g., 65%, 70%, 75%, 80%, 85%, 90%, 95%, etc.) identical to a light chain variable region sequence set forth as SEQ ID NO:2. In another embodiment, an antibody or subsequence includes a sequence at least 60% or more (e.g., 65%, 70%, 75%, 80%, 85%, 90%, 95%, etc.) identical to a heavy chain variable region sequence set forth as SEQ ID NO:1, and a sequence at least 60% or more (e.g., 65%, 70%, 75%, 80%, 85%, 90%, 95%, etc.) identical to a light chain variable region sequence set forth as SEQ ID NO:2. In a further embodiment, an antibody or subsequence includes a sequence at least 80-85%, 85-90%, 90-95%, or 95-100% identical to one or more CDRs in heavy chain variable region sequence set forth as SEQ ID NO:1, or a sequence at least 80-85%, 85-90%, 90-95%, or 95-100% identical to one or more CDRs in a light chain variable region sequence set forth as SEQ ID NO:2 (e.g., amino acids 11-18, 36-43, or 82-100 of SEQ ID NO:1 or amino acids 26-34, 52-58, or 97-103 of SEQ ID NO:2). In various aspects, the antibody or functional fragment thereof binds to an epitope or an antigen to which PM-2 antibody, produced by a cell line DSMZ Deposit No. DSM ACC 2600, or represented by heavy and light chain sequences set forth as SEQ ID NOs:1 and 2 binds (e.g., a protein antigen of about 115 kDa expressed on BXPC-3, MKN or CRL cells); the antibody or functional fragment thereof competes with PM-2 antibody, produced by a cell line DSMZ Deposit No. DSM ACC 2600, or represented by heavy and light chain sequences set forth as SEQ ID NOs:1 and 2, for binding to HT-29, CACO-2, COLO-320, COLO-206F, ASPC-1, BXPC-3 or A549 cells; the antibody or functional fragment thereof binds to an epitope to which PM-2 antibody, produced by a cell line DSMZ Deposit No. DSM ACC 2600, or represented by heavy and light chain sequences set forth as SEQ ID NOs:1 and 2 binds, wherein the epitope is present on a protein antigen of about 115 kDa expressed on BXPC-3, MKN or CRL cells; and the antibody or functional fragment thereof competes with PM-2 antibody, produced by a cell line DSMZ Deposit No. DSM ACC 2600, or represented by heavy and light chain sequences set forth as SEQ ID NOs:1 and 2, for binding to an epitope present on a protein antigen of about 115 kDa expressed on BXPC-3, MKN or CRL cells.

The invention further provides isolated and purified antibodies and functional fragments thereof that have one or more amino acid additions, deletions or substitutions of PM-2 antibody, produced by a cell line DSMZ Deposit No. DSM ACC 2600, or represented by heavy and light chain sequences set forth as SEQ ID NOs:1 and 2. In particular aspects, an antibody or functional fragment has sequence at least 80-85%, 85-90%, 90-95%, or 95-100% identical to a heavy chain variable region sequence set forth as SEQ ID NO:1, or a sequence at least 80-85%, 85-90%, 90-95%, or 95-100% identical to a light chain variable region sequence set forth as SEQ ID NO:2. In further aspects, an antibody or functional fragment has a heavy or light chain sequence with 100% identity to one or more CDRs in a heavy or light chain variable region sequence set forth as SEQ ID NOs:1 and 2 (e.g., amino acids 11-18, 36-43, or 82-100 of SEQ ID NO:1 or amino acids 26-34, 52-58, or 97-103 of SEQ ID NO:2), and has less than 100% identity to a region outside of the CDRs in a heavy or light chain variable region sequence set forth as SEQ ID NOs:1 and 2.

The invention also provides antibodies and functional fragments thereof that have a binding affinity within about 1-5000 fold of the binding affinity of PM-2 antibody, produced by a cell line DSMZ Deposit No. DSM ACC 2600, or represented by heavy and light chain sequences set forth as SEQ ID NOs:1 and 2, for binding to an antigen or a cell (e.g., a neoplastic, cancer, tumor or metastatic cell). In various embodiments, antibodies and functional fragments have a binding affinity within about 1-5000 fold of the binding affinity of PM-2 antibody, produced by a cell line DSMZ Deposit No. DSM ACC 2600, or represented by heavy and light chain sequences set forth as SEQ ID NOs:1 and 2, for binding to a stomach adenocarcinoma, colorectal adenocarcinoma, squamous cell lung carcinoma, lung adenocarcinoma, squamous cell carcinoma of the esophagus, adenocarcinoma of the pancreas, urothel carcinoma of the urinary bladder, renal cell carcinoma of the kidney, adenocarcinoma of the prostate, ductal carcinoma of the breast, lobular carcinoma of the breast, adenocarcinoma of the ovary, adenocarcinoma of the adrenal gland, adenocarcinoma of the endometrium or a uterus cell adenocarcinoma. In additional embodiments, an antibody or functional fragment has a binding affinity within about 1-5000 fold of the binding affinity of PM-2 antibody, produced by a cell line DSMZ Deposit No. DSM ACC 2600, or represented by heavy and light chain sequences set forth as SEQ ID NOs:1 and 2, for binding to HT-29 (ATCC Accession No. HTB-38; DSMZ Accession No. ACC 299), CACO-2 (ATCC Accession No. HBT-37; DSMZ Accession No. ACC 169), COLO-320 (DSMZ Accession No. ACC 144), COLO-206F (DSMZ Accession No. ACC 21), ASPC-1 (ATCC Accession No. CRL-1682), BXPC-3 (ATCC Accession No. CRL-1687) or A549 (ATCC Accession No. CCL-185) cells. In further embodiments, an antibody or functional fragment has a binding affinity within about KD 10⁻⁵ M to about KD 10⁻¹³ M for binding to one or more cells or cell lines set forth herein (e.g., a stomach adenocarcinoma, colorectal adenocarcinoma, squamous cell lung carcinoma, lung adenocarcinoma, squamous cell carcinoma of the esophagus, adenocarcinoma of the pancreas, urothel carcinoma of the urinary bladder, renal cell carcinoma of the kidney, adenocarcinoma of the prostate, ductal carcinoma of the breast, lobular carcinoma of the breast, adenocarcinoma of the ovary, adenocarcinoma of the adrenal gland, adenocarcinoma of the endometrium or a uterus cell adenocarcinoma, etc., or a HT-29 (ATCC Accession No. HTB-38; DSMZ Accession No. ACC 299), CACO-2 (ATCC Accession No. HBT-37; DSMZ Accession No. ACC 169), COLO-320 (DSMZ Accession No. ACC 144), COLO-206F (DSMZ Accession No. ACC 21), ASPC-1 (ATCC Accession No. CRL-1682), BXPC-3 (ATCC Accession No. CRL-1687) or A549 (ATCC Accession No. CCL-185) cells. Such antibodies include, for example, antibody or functional fragment thereof that binds to an epitope or an antigen to which PM-2 antibody, produced by a cell line DSMZ Deposit No. DSM ACC 2600, or represented by heavy and light chain sequences set forth as SEQ ID NOs:1 and 2 binds (e.g., a protein antigen of about 115 kDa expressed on BXPC-3, MKN or CRL cells); antibody or functional fragment thereof competes with PM-2 antibody, produced by a cell line DSMZ Deposit No. DSM ACC 2600, or represented by heavy and light chain sequences set forth as SEQ ID NOs:1 and 2, for binding to HT-29, CACO-2, COLO-320, COLO-206F, ASPC-1, BXPC-3 or A549 cells; antibody or functional fragment thereof that binds to an epitope to which PM-2 antibody, produced by a cell line DSMZ Deposit No. DSM ACC 2600, or represented by heavy and light chain sequences set forth as SEQ ID NOs:1 and 2 binds, wherein the epitope is present on a protein antigen of about 115 kDa expressed on BXPC-3, MKN or CRL cells; and antibody or functional fragment thereof competes with PM-2 antibody, produced by a cell line DSMZ Deposit No. DSM ACC 2600, or represented by heavy and light chain sequences set forth as SEQ ID NOs:1 and 2, for binding to an epitope present on a protein antigen of about 115 kDa expressed on BXPC-3, MKN or CRL cells.

Antibodies of the invention include IgG, IgA, IgM, IgE and IgD. In various aspects, an IgG is an IgG1, IgG2, IgG3, or IgG4.

Antibody functional fragments and subsequences of the invention include functional fragments and subsequences of the various antibodies set forth herein. In a particular embodiment, a functional fragment of PM-2 antibody, produced by a cell line DSMZ Deposit No. DSM ACC 2600, or represented by heavy and light chain sequences set forth as SEQ ID NOs:1 and 2, that competes with PM-2 antibody, produced by a cell line DSMZ Deposit No. DSM ACC 2600, or represented by heavy and light chain sequences set forth as SEQ ID NOs:1 and 2, for binding to a cell, antigen or epitope, or that retains at least partial binding to a cell, antigen or epitope to which PM-2 antibody, produced by a cell line DSMZ Deposit No. DSM ACC 2600, or represented by heavy and light chain sequences set forth as SEQ ID NOs:1 and 2, binds, is provided. In particular aspects, a functional fragment or a subsequence is an Fab, Fab′, F(ab′)₂, Fv, Fd, single-chain Fv (scFv), disulfide-linked Fvs (sdFv), V_{L}, V_{H}, trispecific (Fab₃), bispecific (Fab₂), diabody ((V_{L}-V_{H})₂ or (V_{H}-V_{L})₂), triabody (trivalent), tetrabody (tetravalent), minibody ((scF_{V}-C_{H}3)₂), bispecific single-chain Fv (Bis-scFv), IgGdeltaCH2, scFv-Fc and (scFv)₂-Fc. In additional aspects, a functional fragment or a subsequence of a full length antibody heavy or light chain, or a heavy or light chain variable region, includes one or more CDRs of a heavy or light chain sequence of PM-2 antibody, produced by a cell line DSMZ Deposit No. DSM ACC 2600, or represented by heavy and light chain sequences set forth as SEQ ID NOs:1 and 2 (e.g., amino acids 11-18, 36-43, or 82-100 of SEQ ID NO:1 or amino acids 26-34, 52-58, or 97-103 of SEQ ID NO:2). In further aspects, a functional fragment or a subsequence of a full length antibody heavy or light chain, or a heavy or light chain variable region, has a length from about 20-30, 30-50, 50-100, 100-150, 150-200, 200-250, 250-300, 300-400, 400-500, amino acid residues.

The invention also provides antibodies and subsequences that include a heterologous domain. In one embodiment, a heterologous domain includes a detectable label, tag or cytotoxic agent. In particular aspects, a detectable label or tag is an enzyme, enzyme substrate, ligand, receptor, radionuclide, a T7-, His-, myc-, HA- or FLAG-tag, electron-dense reagent, energy transfer molecule, paramagnetic label, fluorophore, chromophore, chemi-luminescent agent, or a bio-luminescent agent.

The invention moreover provides nucleic acid sequences that encode antibodies and functional fragments thereof. In one embodiment, a nucleic acid sequence is at least 75-100% complementary or identical to a nucleic acid sequence that encodes a heavy or a light chain variable region sequence of PM-2 antibody, produced by a cell line DSMZ Deposit No. DSM ACC 2600, or represented by heavy and light chain sequences set forth as SEQ ID NOs:1 and 2, or a subsequence thereof (e.g., amino acids 11-18, 36-43, or 82-100 of SEQ ID NO:1, or amino acids 26-34, 52-58, or 97-103 of SEQ ID NO:2, such as nucleotides 31-54, 106-129, and 244-300 of SEQ ID NO:3 and nucleotides 76-102, 154-174, or 289-309 of SEQ ID NO:4). In another embodiment, a nucleic acid encodes a subsequence of PM-2 antibody, produced by a cell line DSMZ Deposit No. DSM ACC 2600, or represented by heavy and light chain sequences set forth as SEQ ID NOs:1 and 2 (e.g., amino acids 11-18, 36-43, or 82-100 of SEQ ID NO:1, or amino acids 26-34, 52-58, or 97-103 of SEQ ID NO:2, such as nucleotides 31-54, 106-129, and 244-300 of SEQ ID NO:3 and nucleotides 76-102, 154-174, or 289-309 of SEQ ID NO:4). In particular aspects, a nucleic acid sequence has a length from about 10-20, 20-30, 30-50, 50-100, 100-150, 150-200, 200-250, 250-300, 300-400, 400-500, or 500-1000 nucleotides. In additional aspects, a nucleic acid sequence specifically hybridizes to a nucleic acid that encodes SEQ ID NO:1 or 2, or a subsequence thereof, or specifically hybridizes to a nucleic acid sequence complementary to a nucleic acid that encodes SEQ ID NO:1 or 2, or a subsequence SEQ ID NO:1 or 2 (e.g., nucleotides 31-54, 106-129, and 244-300 of SEQ ID NO:3 and nucleotides 76-102, 154-174, or 289-309 of SEQ ID NO:4). In further aspects, a nucleic acid is an antisense polynucleotide, a small interfering RNA, or a ribozyme nucleic acid that specifically hybridizes to a nucleic acid sequence encoding or complementary to SEQ ID NO:1 or 2 or a subsequence thereof. Antisense polynucleotides, small interfering RNA, and ribozyme polynucleotides can have a length from about 10-20, 20-30, 30-50, 50-100, 100-150, 150-200, 200-250, 250-300, 300-400, 400-500, 500-1000, 1000-2000 nucleotides, and be at least 90% complementary or identical to a nucleic acid sequence that encodes SEQ ID NOs:1 or 2, or a subsequence thereof (e.g., amino acids 11-18, 36-43, or 82-100 of SEQ ID NO:1, or amino acids 26-34, 52-58, or 97-103 of SEQ ID NO:2, such as nucleotides 31-54, 106-129, and 244-300 of SEQ ID NO:3 and nucleotides 76-102, 154-174, or 289-309 of SEQ ID NO:4). In still further aspects, nucleic acid sequence can include an expression control sequence or a vector (e.g., a viral, bacterial, fungal or mammalian vector).

The invention additionally provides isolated and purified cells as well as transformed host cells that express an antibody or subsequence thereof that includes a sequence at least 60% or more (e.g., 65%, 70%, 75%, 80%, 85%, 90%, 95%, etc.) identical to a heavy or light chain variable region sequence set forth as SEQ ID NO:1 or 2, or a sequence at least 60% or more (e.g., 65%, 70%, 75%, 80%, 85%, 90%, 95%, etc.) identical to a heavy or light chain variable region sequence of PM-2 antibody, produced by a cell line DSMZ Deposit No. DSM ACC 2600, or represented by heavy and light chain sequences set forth as SEQ ID NOs:1 and 2. Such cells include eukaryotic and non-eukaryotic cells, which can stably or transiently express antibody or subsequence thereof, or be stably or transiently transformed with the nucleic acid or vector that encodes antibody or subsequence thereof or.

The invention further provides kits. In various embodiments, a kit includes an antibody or functional fragment thereof that competes with PM-2 antibody, produced by a cell line DSMZ Deposit No. DSM ACC 2600, or represented by heavy and light chain sequences set forth as SEQ ID NOs:1 and 2, for binding to an antigen, epitope or to a cell (e.g., a neoplastic, cancer, tumor or metastatic cell). In particular aspects, a kit includes an antibody or functional fragment thereof that competes with PM-2 antibody, produced by a cell line DSMZ Deposit No. DSM ACC 2600, or represented by heavy and light chain sequences set forth as SEQ ID NOs:1 and 2, for binding to a stomach adenocarcinoma, colorectal adenocarcinoma, squamous cell lung carcinoma, lung adenocarcinoma, squamous cell carcinoma of the esophagus, adenocarcinoma of the pancreas, urothel carcinoma of the urinary bladder, renal cell carcinoma of the kidney, adenocarcinoma of the prostate, ductal carcinoma of the breast, lobular carcinoma of the breast, adenocarcinoma of the ovary, adenocarcinoma of the adrenal gland, adenocarcinoma of the endometrium or a uterus cell adenocarcinoma. In an additional embodiment, a kit includes an antibody or functional fragment thereof that competes with PM-2 antibody, produced by a cell line DSMZ Deposit No. DSM ACC 2600, or represented by heavy and light chain sequences set forth as SEQ ID NOs:1 and 2, for binding to HT-29 (ATCC Accession No. HTB-38; DSMZ Accession No. ACC 299), CACO-2 (ATCC Accession No. HBT-37; DSMZ Accession No. ACC 169), COLO-320 (DSMZ Accession No. ACC 144), COLO-206F (DSMZ Accession No. ACC 21), ASPC-1 (ATCC Accession No. CRL-1682), BXPC-3 (ATCC Accession No. CRL-1687) or A549 (ATCC Accession No. CCL-185) cells.

Kits of the invention also include antibodies and functional fragments that bind to cells, antigen or an epitope that PM-2 antibody, produced by a cell line DSMZ Deposit No. DSM ACC 2600, or represented by heavy and light chain sequences set forth as SEQ ID NOs:1 and 2, binds. In one embodiment, a kit includes an antibody or functional fragment that binds to an adenocarcinoma cell or a squamous cell carcinoma to which PM-2 antibody, produced by a cell line DSMZ Deposit No. DSM ACC 2600, or represented by heavy and light chain sequences set forth as SEQ ID NOs:1 and 2, binds, such as a stomach adenocarcinoma cell, a lung adenocarcinoma cell, a pancreas adenocarcinoma cell, a colon adenocarcinoma cell, a breast adenocarcinoma cell, an esophagus squamous cell carcinoma, to which PM-2 antibody, produced by a cell line DSMZ Deposit No. DSM ACC 2600, or represented by heavy and light chain sequences set forth as SEQ ID NOs:1 and 2, binds. In another embodiment, a kit includes an antibody or functional fragment binds to a stomach adenocarcinoma, colorectal adenocarcinoma, squamous cell lung carcinoma, lung adenocarcinoma, squamous cell carcinoma of the esophagus, adenocarcinoma of the pancreas, urothel carcinoma of the urinary bladder, renal cell carcinoma of the kidney, adenocarcinoma of the prostate, ductal carcinoma of the breast, lobular carcinoma of the breast, adenocarcinoma of the ovary, adenocarcinoma of the adrenal gland, adenocarcinoma of the endometrium or a uterus cell adenocarcinoma to which PM-2 antibody, produced by a cell line DSMZ Deposit No. DSM ACC 2600, or represented by heavy and light chain sequences set forth as SEQ ID NOs:1 and 2, binds. In an additional embodiment, a kit includes an antibody or functional fragment that binds to a HT-29 (ATCC Accession No. HTB-38; DSMZ Accession No. ACC 299), CACO-2 (ATCC Accession No. HBT-37; DSMZ Accession No. ACC 169), COLO-320 (DSMZ Accession No. ACC 144), COLO-206F (DSMZ Accession No. ACC 21), ASPC-1 (ATCC Accession No. CRL-1682), BXPC-3 (ATCC Accession No. CRL-1687) or A549 (ATCC Accession No. CCL-185) cells that PM-2 antibody, produced by a cell line DSMZ Deposit No. DSM ACC 2600, or represented by heavy and light chain sequences set forth as SEQ ID NOs:1 and 2, binds.

Kits of the invention further include antibodies and functional fragments that include a heavy or light chain variable region sequence with about 60% or more identity to a heavy or light chain sequence variable regions of PM-2 antibody, produced by a cell line DSMZ Deposit No. DSM ACC 2600, or represented by heavy and light chain sequences set forth as SEQ ID NOs:1 and 2. In one embodiment, a kit includes an antibody or subsequence thereof with a sequence at least 60% or more (e.g., 65%, 70%, 75%, 80%, 85%, 90%, 95%, etc.) identical to a heavy chain variable region sequence set forth as SEQ ID NO:1, or to a sequence at least 60% or more (e.g., 65%, 70%, 75%, 80%, 85%, 90%, 95%, etc.) identical to a light chain variable region sequence set forth as SEQ ID NO:2. In another embodiment, a kit includes an antibody or subsequence with a sequence at least 60% or more (e.g., 65%, 70%, 75%, 80%, 85%, 90%, 95%, etc.) identical to a heavy chain variable region sequence set forth as SEQ ID NO:1, and to a sequence at least 60% or more (e.g., 65%, 70%, 75%, 80%, 85%, 90%, 95%, etc.) identical to a light chain variable region sequence set forth as SEQ ID NO:2. In further embodiments, a kit includes an antibody or subsequence with a sequence at least 80-85%, 85-90%, 90-95%, 95-100% identical to one or more CDRs in heavy chain variable region sequence set forth as SEQ ID NO:1 (e.g., amino acids 11-18, 36-43, or 82-100 of SEQ ID NO:1), or a sequence at least 80-85%, 85-90%, 90-95%, 95-100% identical to one or more CDRs in a light chain variable region sequence set forth as SEQ ID NO:2 (e.g., amino acids 26-34, 52-58, or 97-103 of SEQ ID NO:2).

In additional embodiments, a kit also includes an anti-cell proliferative or immune enhancing treatment or therapeutic agent, or an anti-neoplastic, anti-cancer or anti-tumor or anti-metastatic agent, or an article of manufacture (e.g., for delivering the antibody, anti-cell proliferative or immune enhancing treatment or therapy into a subject locally, regionally or systemically). In particular aspects, the instructions are for treating undesirable cell proliferation or a cell proliferative disorder (e.g., a neoplasia, tumor cancer or metastasis).

The invention yet additionally provides pharmaceutical compositions. In one embodiment, a composition includes an antibody or functional fragment and a pharmaceutically acceptable carrier or excipient. In another embodiment, a composition includes an antibody that competes with PM-2 antibody, produced by a cell line DSMZ Deposit No. DSM ACC 2600, or represented by heavy and light chain sequences set forth as SEQ ID NOs:1 and 2, for binding to a cell, antigen or epitope, or that binds to a cell, antigen or epitope to which PM-2 antibody, produced by a cell line DSMZ Deposit No. DSM ACC 2600, or represented by heavy and light chain sequences set forth as SEQ ID NOs:1 and 2, binds, or that includes a heavy or light chain variable region sequence with about 60% or more identity to a heavy or light chain sequence variable regions as set forth in SEQ ID NOs:1 or 2 or a sequence at least 80-85%, 85-90%, 90-95%, 95-100% identical to one or more CDRs in a heavy chain or light chain variable region sequence set forth as SEQ ID NO:1 or 2 (e.g., amino acids 11-18, 36-43, or 82-100 of SEQ ID NO:1, or amino acids 26-34, 52-58, or 97-103 of SEQ ID NO:2), and a pharmaceutically acceptable carrier or excipient.

Antibodies, functional fragments and modified forms are useful for treating a subject in need of treatment. The invention therefore provides methods of using antibodies and functional fragments in treatment (e.g., therapeutic or prophylactic) of a subject having or at risk of having undesirable cell proliferation, such as a cell proliferative or hyperproliferative disorder. In one embodiment, a method includes administering an antibody or functional fragment (e.g., a PM-2 antibody, produced by a cell line DSMZ Deposit No. DSM ACC 2600, or represented by heavy and light chain sequences set forth as SEQ ID NOs:1 and 2) to a subject having or at risk of having undesirable cell proliferation (e.g., a cell proliferative disorder) an amount effective to treat undesirable cell proliferation. In particular aspects, a cell proliferative disorder is a metastatic or non-metastatic, solid or liquid neoplasia, malignancy, tumor or cancer. In various aspects, undesirable cell proliferation (e.g., a cell proliferative disorder) affects or is present at least in part in brain, head or neck, breast, esophagus, mouth, nasopharynx, nose or sinuses, stomach, duodenum, ileum, jejunum, lung, liver, pancreas, kidney, adrenal gland, thyroid, bladder, colon, rectum, prostate, uterus, endometrium, cervix, ovary, bone marrow, lymph, blood, bone, testes, skin or muscle, or hematopoetic system. In additional aspects, undesirable cell proliferation (e.g., a cell proliferative disorder) includes a neoplasia, tumor, cancer or metastasis that affects or is at least in part present in breast, lung, thyroid, head and neck, nasopharynx, nose or sinuses, brain, spine, adrenal gland, lymph, gastrointestinal tract, mouth, esophagus, stomach, duodenum, ileum, jejunum, small intestine, colon, rectum, genito-urinary tract, uterus, endometrium, ovary, cervix, bladder, testicle, penis, prostate, kidney, pancreas, adrenal gland, liver, bone, bone marrow, lymph, blood, muscle, skin or is hematopoetic. In further particular aspects, a neoplasia, tumor, cancer or metastasis is a sarcoma, carcinoma, adenocarcinoma, melanoma, myeloma, blastoma, glioma, lymphoma leukemia. In additional particular aspects, a neoplasia, tumor or cancer is a stomach adenocarcinoma, colorectal adenocarcinoma, squamous cell lung carcinoma, lung adenocarcinoma, squamous cell carcinoma of the esophagus, adenocarcinoma of the pancreas, urothel carcinoma of the urinary bladder, renal cell carcinoma of the kidney, adenocarcinoma of the prostate, ductal carcinoma of the breast, lobular carcinoma of the breast, adenocarcinoma of the ovary, adenocarcinoma of the adrenal gland, adenocarcinoma of the endometrium or a uterine adenocarcinoma, or a metastasis thereof.

In another embodiment, a method includes administering an antibody or functional fragment (e.g., a PM-2 antibody, produced by a cell line DSMZ Deposit No. DSM ACC 2600, or represented by heavy and light chain sequences set forth as SEQ ID NOs:1 and 2) to a subject having or at risk of having a metastasis an amount effective to reduce or inhibit spread or dissemination of a tumor, cancer or neoplasia to other sites, locations or regions within the subject. In various aspects, a method reduces or inhibits metastasis of a primary tumor or cancer to one or more other sites, the formation or establishment of a metastasis at one or more other sites, thereby inhibiting or reducing tumor or cancer relapse or tumor or cancer progression. In further aspects, a method reduces or inhibits growth, proliferation, mobility or invasiveness of tumor or cancer cells that potentially or do develop metastases (e.g., disseminated tumor cells); reduces or inhibits formation or establishment of metastases arising from a primary tumor or cancer to one or more other sites, locations or regions distinct from the primary tumor or cancer; reduces or inhibits growth or proliferation of a metastasis at one or more other sites, locations or regions distinct from the primary tumor or cancer after the metastasis has formed or has been established; or reduces or inhibits formation or establishment of additional metastasis after the metastasis has been formed or established.

In further particular aspects, a neoplasia, tumor or cancer, or metastasis is progressively worsening or is in remission. In still additional aspects, treatment results in alleviating or ameliorating one or more adverse physical symptoms associated with a cell proliferative disorder, or a neoplasia, tumor or cancer, or reduces or decreases neoplasia, tumor or cancer volume, inhibits or prevents an increase in neoplasia, tumor or cancer volume, inhibits neoplasia, tumor or cancer progression or worsening, stimulates neoplasia, tumor or cancer cell lysis or apoptosis, or inhibits, reduces or decreases neoplasia, tumor or cancer proliferation or metastasis, or prolongs or extends lifespan of the subject, or improves the quality of life of the subject.

Methods include administration to a subject locally, regionally, or systemically. Exemplary subjects (e.g., mammals such as humans) include candidates for, and those undergoing, or having undergone an anti-cell proliferative or anti-hyperproliferative disorder (e.g., anti-neoplastic, anti-tumor, anti-cancer or anti-metastasis) or immune-enhancing treatment or therapy.

The invention yet also provides combined methods for treating a disorder in a subject in need of treatment. In one embodiment, a method includes administering to a subject an antibody that competes with PM-2 antibody, produced by a cell line DSMZ Deposit No. DSM ACC 2600, or represented by heavy and light chain sequences set forth as SEQ ID NOs:1 and 2, for binding of to a cell, or binds to a cell to which PM-2 antibody, produced by a cell line DSMZ Deposit No. DSM ACC 2600, or represented by heavy and light chain sequences set forth as SEQ ID NOs:1 and 2, binds and an anti-cell proliferative or immune-enhancing treatment or therapy to a subject (e.g., prior to, substantially contemporaneously with or following each other). In various aspects, an anti-cell proliferative or immune-enhancing treatment or therapy includes surgical resection, radiotherapy, radiation therapy, chemotherapy, immunotherapy, hyperthermia, an alkylating agent, anti-metabolite, plant extract, plant alkaloid, nitrosourea, hormone, nucleoside or nucleotide analogue, a lymphocyte, plasma cell, macrophage, dendritic cell, NK cell or B-cell, an antibody, a cell growth factor, a cell survival factor, a cell differentiative factor, a cytokine, an interferon or a chemokine.

Antibodies and functional fragments thereof are useful for detecting, screening for and identifying the presence of cells that bind to PM-2 antibody, produced by a cell line DSMZ Deposit No. DSM ACC 2600, or represented by heavy and light chain sequences set forth as SEQ ID NOs:1 and 2, or antigen that binds to PM-2 antibody, produced by a cell line DSMZ Deposit No. DSM ACC 2600, or represented by heavy and light chain sequences set forth as SEQ ID NOs:1 and 2. The invention therefore provides methods for detecting or screening for cells, antigens and epitopes that bind to PM-2 antibody, produced by a cell line DSMZ Deposit No. DSM ACC 2600, or represented by heavy and light chain sequences set forth as SEQ ID NOs:1 and 2, methods for identifying a subject that is amenable to treatment in accordance with the methods of the invention. In one embodiment, a method includes contacting a biological material or sample with an antibody or functional fragment under conditions allowing binding between antibody or functional fragment and cell, antigen or epitope that binds to PM-2 antibody, produced by a cell line DSMZ Deposit No. DSM ACC 2600, or represented by heavy and light chain sequences set forth as SEQ ID NOs:1 and 2, and assaying for binding of the antibody or functional fragment to a cell or antigen that binds to PM-2 antibody, produced by a cell line DSMZ Deposit No. DSM ACC 2600, or represented by heavy and light chain sequences set forth as SEQ ID NOs:1 and 2. The binding of the antibody or functional fragment to a cell, antigen or epitope that binds to PM-2 antibody, produced by a cell line DSMZ Deposit No. DSM ACC 2600, or represented by heavy and light chain sequences set forth as SEQ ID NOs:1 and 2, indicates that the biological material contains the cell, antigen or epitope that binds to PM-2 antibody, produced by a cell line DSMZ Deposit No. DSM ACC 2600, or represented by heavy and light chain sequences set forth as SEQ ID NOs:1 and 2. In one aspect, the biological material or sample is obtained from a mammalian (e.g., primate, such as a human) subject.

The invention moreover provides methods for diagnosing a subject having or at increased risk of having undesirable cell proliferation or a cell proliferative disorder (e.g., neoplasia, tumor or cancer, or metastasis). In various embodiments, a method includes providing a biological material or sample from a subject, contacting the biological material or sample with an antibody or functional fragment that competes with PM-2 antibody, produced by a cell line DSMZ Deposit No. DSM ACC 2600, or represented by heavy and light chain sequences set forth as SEQ ID NOs:1 and 2, for binding or an antibody or functional fragment that binds to a cell, antigen or epitope to which PM-2 antibody, produced by a cell line DSMZ Deposit No. DSM ACC 2600, or represented by heavy and light chain sequences set forth as SEQ ID NOs:1 and 2, binds, or an antibody or functional fragment that includes a heavy or light chain variable region sequence with about 60% or more identity to a heavy or light chain sequence variable regions of PM-2 antibody, produced by a cell line DSMZ Deposit No. DSM ACC 2600, or represented by heavy and light chain sequences set forth as SEQ ID NOs:1 and 2, under conditions allowing binding of the antibody or functional fragment, and assaying for binding of the antibody to a cell, antigen or epitope that binds to PM-2 antibody, produced by a cell line DSMZ Deposit No. DSM ACC 2600, or represented by heavy and light chain sequences set forth as SEQ ID NOs:1 and 2. In particular aspects, the methods for diagnosing a subject identify those that have or are at increased risk of having undesirable cell proliferation or a cell proliferative disorder (e.g., neoplasia, tumor or cancer, or metastasis). In one aspect, the biological material or sample is obtained from a mammalian (e.g., primate, such as a human) subject. In additional aspects, the biological material or sample comprises a biopsy, such as a lung, pancreas, stomach, breast, esophageal, ovarian or uterine biopsy.

### DESCRIPTION OF DRAWING

FIG. 1 shows that PM-2 monoclonal antibody induces apoptosis in BXPC-3 human pancreatic carcinoma cells after a 24 hour incubation period when compared to a negative control. The Y-axis is the difference between the absorbance at 415 nm and at the 490 nm reference wavelength (A₄₁₅-A₄₉₀). The concentration of PM-2 antibody was either 6 μg or 12 μg/ml in supernatant and the negative control is RPMI 1460 medium.

### DETAILED DESCRIPTION

The invention is based, at least in part, on antibodies that bind to various neoplastic, cancer, tumor and metastatic cells. A non-limiting exemplary antibody is designated PM-2 antibody, produced by a cell line DSMZ Deposit No. DSM ACC 2600, deposited on Jul. 2, 2003 at the German Collection of Microorganisms and Cell Cultures (“DSMZ”—Deutsche Sammlung von Mikroorganismen und Zellkulturen GmbH, Mascheroder Weg lb, 38124 Braunschweig, Germany) under the terms of the Budapest Treaty, or represented by heavy and light chain sequences set forth as SEQ ID NOs:1 and 2. PM-2 antibody, produced by a cell line DSMZ Deposit No. DSM ACC 2600, or represented by heavy and light chain sequences set forth as SEQ ID NOs:1 and 2, is a human IgM antibody that specifically binds to various neoplastic, cancer, tumor and metastatic cells. PM-2 therefore binds to an antigen expressed on various neoplastic, cancer, tumor and metastatic cells. PM-2 is able to inhibit or reduce proliferation of various neoplastic, cancer, tumor and metastatic cells. PM-2 is also able to stimulate or induce apoptosis of various neoplastic, cancer, tumor and metastatic cells.

Antibodies of the invention include polyclonal and monoclonal antibodies. Antibodies are proteins which include amino acids, or “residues,” covalently linked by an amide bond or equivalent. The term “monoclonal,” when used in reference to an antibody refers to an antibody that is based upon, obtained from or derived from a single clone, including any eukaryotic, prokaryotic, or phage clone. A “monoclonal” antibody is therefore defined herein structurally, and not the method by which it is produced.

Antibodies of the invention can belong to any antibody class, IgM, IgG, IgE, IgA, IgD, or subclass. Exemplary subclasses for IgG are IgG₁, IgG₂, IgG₃ and IgG₄.

Antibodies of the invention can have kappa or lambda light chain sequences, either full length as in naturally occurring antibodies, mixtures thereof (i.e., fusions of kappa and lambda chain sequences), and subsequences/fragments thereof. Naturally occurring antibody molecules contain two kappa or two lambda light chains. The primary difference between kappa and lambda light chains is in the sequences of the constant region.

The amino acid and nucleic acid sequences of PM-2 antibody, represented by heavy and light chain sequences, SEQ ID NOs:1-4, are as follows:

| PM-2 Heavy chain variable region amino acid and nucleic acid sequences, | |
|---|---|
| SEQ ID NOs:1 and 3 | |
| CDR1 | |
| ggg tcc ctg aga ctc tcc tgt gca gcc tct g̅g̅a̅ t̅t̅c̅ a̅c̅c̅ t̅t̅t̅ a̅g̅c̅ a̅g̅c̅ t̅a̅t̅ g̅c̅c̅ atg agc | 60 |
| Gly Ser Leu Arg Leu Ser Cys Ala Ala Ser Gly Phe Thr Phe Ser Ser Tyr Ala Met Ser | |
| 1 5 10 15 20 | |
| CDR2 | |
| tgg gtc cgc cag gct cca ggg aag ggg ctg gag tgg gtc tca gct a̅t̅t̅ a̅g̅t̅ g̅g̅t̅ a̅g̅t̅ g̅g̅t̅ | 120 |
| Trp val Arg Gln Ala Pro Gly Lys Gly Leu Gln Trp val Ser Ala Ile Aer Gly Ser Gly | |
| 25 30 35 40 | |
| g̅t̅t̅ a̅g̅t̅ a̅c̅a̅ tac tac gca gac tcc gtg aag ggc cgg ttc acc atc tcc aga gac aat tcc | 180 |
| Gly Ser Thr Tyr Tyr Ala Asp Ser Val Lys Gly Arg Phe Thr Ile Ser Arg Asp Asn Ser | |
| 45 50 55 60 | |
| aag aac acg ctg tat ctg caa atg aac agc ctg aga gcc gag gac acg gcc gta tat tac | 240 |
| Lys Asn Thr Leu Tyr Leu Gln Met Asn Ser Leu Arg Ala Glu Asp Thr Ala Val Tyr Tyr | |
| 65 70 75 80 | |
| CDR3 | |
| tgt g̅c̅g̅ a̅a̅a̅ g̅g̅t̅ g̅g̅g̅ g̅c̅c̅ g̅a̅a̅ g̅g̅c̅ t̅g̅g̅ t̅a̅c̅ g̅a̅g̅ t̅a̅c̅ t̅a̅c̅ t̅a̅c̅ t̅a̅c̅ t̅a̅c̅ g̅g̅t̅ a̅t̅g̅ g̅a̅c̅ g̅t̅c̅ | 300 |
| Cys Ala Lys Gly Gly Ala Glu Gly Trp Tyr Glu Tyr Tyr Tyr Tyr Tyr Gly Met Asp Val | |
| 85 90 95 100 | |
| tgg ggc caa ggg acc ctg gtc | 321 |
| Trp Gly Gln Gly Thr Leu Val | |
| 105 | |
| PM-2 Light chain variable region amino acid and nucleic acid sequences, | |
| SEQ ID NOs:2 and 4 | |
| cag tct gcc ctg act cag cct gct tcc ctc tct gca tct cct gga gca tca ggc agt ctc | 60 |
| Gln Ser Ala Leu Thr Gln Pro Ala Ser Leu Ser Ala Ser Pro Gly Ala Ser Ala Ser Leu | |
| 1 5 10 15 20 | |
| CDR1 | |
| acc tgc acc ttg cgc a̅g̅t̅ g̅g̅c̅ a̅t̅c̅ a̅a̅t̅ g̅t̅t̅ g̅g̅t̅ a̅c̅c̅ t̅a̅c̅ a̅g̅g̅ ata tac tgg tac cag cag | 120 |
| Thr Cys Thr Leu Arg Ser Gly Ile Asn Val Gly Thr Tyr Arg Ile Tyr Trp Tyr Gln Gln | |
| 25 30 35 40 | |
| CDR2 | |
| aag cca ggg agt cct ccc cag tat ctc ctg agg t̅a̅c̅ a̅a̅a̅ t̅c̅a̅ g̅a̅c̅ t̅c̅a̅ g̅a̅t̅ a̅a̅g̅ cag aag | 180 |
| Lys Pro Gly Ser Pro Pro Gln Tyr Leu Leu Arg Tyr Lys Ser Asp Ser Asp Lys Gln Lys | |
| 45 50 55 60 | |
| ggc tct gga gtc ccc agc cgc ttc tct gga tcc aaa gat gct tcg gcc aat gca ggg att | 240 |
| Gly Ser Gly Val Pro Ser Arg Phe Ser Gly Ser Lys Asp Ala Ser Ala Asn Ala Gly Ile | |
| 65 70 75 80 | |
| CDR3 | |
| tta ctc atc tct ggg ctc cag tct gag gat gag gct gac tat tac tgt a̅t̅g̅ a̅t̅t̅ t̅g̅g̅ c̅a̅c̅ | 300 |
| Leu Leu Ile Ser Gly Leu Gln Ser Gln Asp Glu Ala Asp Tyr Tyr Cys Met Ile Trp His | |
| 85 90 95 100 | |
| a̅g̅c̅ a̅g̅c̅ g̅c̅t̅ tgg gtg ttc ggc gga ggg acc aag ctg acc gtc cta ggt | 348 |
| Ser Ser Ala Trp Val Phe Gly Gly Gly Thr Lys Leu Thr Val Leu Gly | |
| 105 110 115 | |

Predicted CDRs, of which there are three in each of heavy and light chain sequence set forth as SEQ ID NOs:1 and 2, are conveniently denoted as LC-CDR1, LC-CDR2 and LC-CDR3; and HC-CDR1, HC-CDR2 and HC-CDR3. The CDRs of heavy and light chains are predicted to be located at amino acids 11-18, 36-43, or 82-100 of SEQ ID NO:1, and amino acids 26-34, 52-58, or 97-103 of SEQ ID NO:2, respectively, which correspond to nucleotides 31-54, 106-129, and 244-300 of SEQ ID NO:3, and nucleotides 76-102, 154-174, or 289-309 of SEQ ID NO:4.

In accordance with the invention, there are provided isolated and purified antibodies and functional (e.g., cell, antigen or epitope binding) fragments structurally and/or functionally related to PM-2 antibody, produced by a cell line DSMZ Deposit No. DSM ACC 2600, or represented by heavy and light chain sequences set forth as SEQ ID NOs:1 and 2, respectively. In various embodiments, antibodies and functional fragments compete with PM-2 antibody, produced by a cell line DSMZ Deposit No. DSM ACC 2600, or represented by heavy and light chain sequences set forth as SEQ ID NOs:1 and 2, for binding to a cell, antigen or epitope. In additional embodiments, antibodies and functional fragments compete with PM-2 antibody, produced by a cell line DSMZ Deposit No. DSM ACC 2600, or represented by heavy and light chain sequences set forth as SEQ ID NOs:1 and 2, for binding of to an adenocarcinoma cell or a squamous cell carcinoma. In further embodiments, antibodies and functional fragments compete with PM-2 antibody, produced by a cell line DSMZ Deposit No. DSM ACC 2600, or represented by heavy and light chain sequences set forth as SEQ ID NOs:1 and 2, for binding of to one or more of a stomach adenocarcinoma, colorectal adenocarcinoma, squamous cell lung carcinoma, lung adenocarcinoma, squamous cell carcinoma of the esophagus, adenocarcinoma of the pancreas, urothel carcinoma of the urinary bladder, renal cell carcinoma of the kidney, adenocarcinoma of the prostate, ductal carcinoma of the breast, lobular carcinoma of the breast, adenocarcinoma of the ovary, adenocarcinoma of the adrenal gland, adenocarcinoma of the endometrium or a uterus cell adenocarcinoma. In yet additional embodiments, antibodies and functional fragments compete with PM-2 antibody, produced by a cell line DSMZ Deposit No. DSM ACC 2600, or represented by heavy and light chain sequences set forth as SEQ ID NOs:1 and 2, for binding to a stomach adenocarcinoma, colorectal adenocarcinoma, squamous cell lung carcinoma, lung adenocarcinoma, squamous cell carcinoma of the esophagus, adenocarcinoma of the pancreas, urothel carcinoma of the urinary bladder, renal cell carcinoma of the kidney, adenocarcinoma of the prostate, ductal carcinoma of the breast, lobular carcinoma of the breast, adenocarcinoma of the ovary, adenocarcinoma of the adrenal gland, adenocarcinoma of the endometrium or a uterus cell adenocarcinoma. In still further embodiments, antibodies and functional fragments compete with PM-2 antibody, produced by a cell line DSMZ Deposit No. DSM ACC 2600, or represented by heavy and light chain sequences set forth as SEQ ID NOs:1 and 2, for binding to HT-29 (ATCC Accession No. HTB-38; DSMZ Accession No. ACC 299), CACO-2 (ATCC Accession No. HBT-37; DSMZ Accession No. ACC 169), COLO-320 (DSMZ Accession No. ACC 144), COLO-206F (DSMZ Accession No. ACC 21), ASPC-1 (ATCC Accession No. CRL-1682), BXPC-3 (ATCC Accession No. CRL-1687) or A549 (ATCC Accession No. CCL-185) cells. In particular aspects, antibodies and functional fragments competitively inhibit binding of PM-2 antibody, produced by a cell line DSMZ Deposit No. DSM ACC 2600, or represented by heavy and light chain sequences set forth as SEQ ID NOs:1 and 2, to a cell or antigen by at least 30%, 40%, 50%, 60%, 70%, 80%, 90% or more.

In accordance with the invention, there are also provided antibodies and functional fragments that bind to a cell, antigen or epitope that PM-2 antibody, produced by a cell line DSMZ Deposit No. DSM ACC 2600, or represented by heavy and light chain sequences set forth as SEQ ID NOs:1 and 2, binds. In one embodiment, an isolated or purified antibody or functional fragment thereof binds to a cell, antigen or epitope that PM-2 antibody, produced by a cell line DSMZ Deposit No. DSM ACC 2600, or represented by heavy and light chain sequences set forth as SEQ ID NOs:1 and 2, binds. In particular aspects, the antibody or functional fragment thereof binds to a cell, antigen or epitope present on an adenocarcinoma cell or a squamous cell carcinoma to which PM-2 antibody, produced by a cell line DSMZ Deposit No. DSM ACC 2600, or represented by heavy and light chain sequences set forth as SEQ ID NOs:1 and 2, binds. In additional particular aspects, the antibody or functional fragment thereof binds to a cell, antigen or epitope present on one or more of a stomach adenocarcinoma, colorectal adenocarcinoma, squamous cell lung carcinoma, lung adenocarcinoma, squamous cell carcinoma of the esophagus, adenocarcinoma of the pancreas, urothel carcinoma of the urinary bladder, renal cell carcinoma of the kidney, adenocarcinoma of the prostate, ductal carcinoma of the breast, lobular carcinoma of the breast, adenocarcinoma of the ovary, adenocarcinoma of the adrenal gland, adenocarcinoma of the endometrium or a uterus cell adenocarcinoma, to which PM-2 antibody, produced by a cell line DSMZ Deposit No. DSM ACC 2600, or represented by heavy and light chain sequences set forth as SEQ ID NOs:1 and 2, binds. In further particular aspects, the antibody or functional fragment thereof binds to a cell, antigen or epitope present on a HT-29 (ATCC Accession No. HTB-38; DSMZ Accession No. ACC 299), CACO-2 (ATCC Accession No. HBT-37; DSMZ Accession No. ACC 169), COLO-320 (DSMZ Accession No. ACC 144), COLO-206F (DSMZ Accession No. ACC 21), ASPC-1 (ATCC Accession No. CRL-1682), BXPC-3 (ATCC Accession No. CRL-1687) or A549 (ATCC Accession No. CCL-185) cell, to which PM-2 antibody, produced by a cell line DSMZ Deposit No. DSM ACC 2600, or represented by heavy and light chain sequences set forth as SEQ ID NOs:1 and 2, binds.

The term “bind,” or “binding,” when used in reference to an antibody or functional fragment, means that the antibody or functional fragment interacts at the molecular level with a corresponding epitope (antigenic determinant) present on a cell or an antigen. Epitopes of antigens that comprise amino acids typically include relatively short sequences, e.g. about five to 15 amino acids in length. Epitopes can be contiguous or non-contiguous. A non-contiguous amino acid sequence epitope forms due to protein folding. Techniques for identifying epitopes are known to the skilled artisan and include screening overlapping oligopeptides for binding to antibody (for example, U.S. Pat. No. 4,708,871), phage display peptide library kits, which are commercially available for epitope mapping (New England BioLabs). Epitopes may also be identified by inference when epitope length peptide sequences are used to immunize animals from which antibodies that bind to the peptide sequence are obtained and can be predicted using computer programs, such as BEPITOPE (Odorico et al., J. Mol. Recognit. 16:20 (2003)).

The invention further provides antibodies and functional fragments that inhibit, decrease or reduce cell growth or proliferation, or stimulate or induce cell death, lysis or apoptosis. In particular embodiments, binding of PM-2 antibody, produced by a cell line DSMZ Deposit No. DSM ACC 2600, or represented by heavy and light chain sequences set forth as SEQ ID NOs:1 and 2, to a neoplastic, tumor or cancer, or metastasis cell inhibits, decreases or reduces cell growth or proliferation, or stimulates or induces cell death, lysis or apoptosis. In another embodiment, binding of PM-2 antibody, produced by a cell line DSMZ Deposit No. DSM ACC 2600, or represented by heavy and light chain sequences set forth as SEQ ID NOs:1 and 2, to a stomach adenocarcinoma, colorectal adenocarcinoma, squamous cell lung carcinoma, lung adenocarcinoma, squamous cell carcinoma of the esophagus, adenocarcinoma of the pancreas, urothel carcinoma of the urinary bladder, renal cell carcinoma of the kidney, adenocarcinoma of the prostate, ductal carcinoma of the breast, lobular carcinoma of the breast, adenocarcinoma of the ovary, adenocarcinoma of the adrenal gland, adenocarcinoma of the endometrium or a uterus cell adenocarcinoma inhibits, decreases or reduces cell growth or proliferation, or stimulates or induces cell death, lysis or apoptosis. In a further embodiment, binding of PM-2 antibody, produced by a cell line DSMZ Deposit No. DSM ACC 2600, or represented by heavy and light chain sequences set forth as SEQ ID NOs:1 and 2, to HT-29 (ATCC Accession No. HTB-38; DSMZ Accession No. ACC 299), CACO-2 (ATCC Accession No. HBT-37; DSMZ Accession No. ACC 169), COLO-320 (DSMZ Accession No. ACC 144), COLO-206F (DSMZ Accession No. ACC 21), ASPC-1 (ATCC Accession No. CRL-1682), BXPC-3 (ATCC Accession No. CRL-1687) or A549 (ATCC Accession No. CCL-185) cells.

The invention moreover provides of antibodies and functional fragments that are structurally and/or functionally related to PM-2 antibody, produced by a cell line DSMZ Deposit No. DSM ACC 2600, or represented by heavy and light chain sequences set forth as SEQ ID NOs:1 and 2, which includes a heavy or light chain variable region sequence that exhibits a degree of identity to SEQ ID NOs:1 or 2, or that exhibits a degree of identity to a sequence within SEQ ID NOs:1 or 2 (e.g., one or more CDRs, such as amino acids 11-18, 36-43, or 82-100 of SEQ ID NO:1, or amino acids 26-34, 52-58, or 97-103 of SEQ ID NO:2). In particular embodiments, antibodies and functional fragments include a heavy or a light chain variable region sequence with about 60% or more identity to a heavy or light chain sequence variable region of PM-2 antibody, produced by a cell line DSMZ Deposit No. DSM ACC 2600, or represented by heavy and light chain sequences set forth as SEQ ID NOs:1 and 2, or a sequence within PM-2 antibody, produced by a cell line DSMZ Deposit No. DSM ACC 2600, or represented by heavy and light chain sequences set forth as SEQ ID NOs:1 and 2 (e.g., one or more CDRs, such as amino acids 11-18, 36-43, or 82-100 of SEQ ID NO:1, or amino acids 26-34, 52-58, or 97-103 of SEQ ID NO:2). In other particular embodiments, antibodies or functional fragments include a heavy or a light chain with at least 65%, 70%, 75%, 80%, 85%, 90%, 95%, or more identity to a heavy chain variable region sequence of PM-2 antibody, produced by a cell line DSMZ Deposit No. DSM ACC 2600, or represented by heavy and light chain sequences set forth as SEQ ID NOs:1 and 2, or a sequence within PM-2 antibody, produced by a cell line DSMZ Deposit No. DSM ACC 2600, or represented by heavy and light chain sequences set forth as SEQ ID NOs:1 and 2 (e.g., one or more CDRs, such as amino acids 11-18, 36-43, or 82-100 of SEQ ID NO:1, or amino acids 26-34, 52-58, or 97-103 of SEQ ID NO:2). In additional particular embodiments, antibodies or functional fragments include a heavy or a light chain variable region sequence with at least 80-85%, 85-90%, 90-95%, 95-100% identity to one or more CDRs in PM-2 antibody, produced by a cell line DSMZ Deposit No. DSM ACC 2600, or represented by heavy and light chain sequences set forth as SEQ ID NOs:1 and 2 (e.g., amino acids 11-18, 36-43, or 82-100 of SEQ ID NO:1, or amino acids 26-34, 52-58, or 97-103 of SEQ ID NO:2). In a particular aspect, an antibody or a functional fragment thereof includes a heavy or a light chain variable region sequence with 95-100% identity to one, two or three CDRs in each heavy or light chain variable region sequences in PM-2 antibody, produced by a cell line DSMZ Deposit No. DSM ACC 2600, or represented by heavy and light chain sequences set forth as SEQ ID NOs:1 and 2 (e.g., amino acids 11-18, 36-43, or 82-100 of SEQ ID NO:1 or amino acids 26-34, 52-58, or 97-103 of SEQ ID NO:2).

Antibodies and functional fragments of the invention therefore include those with at least partial sequence identity to PM-2 antibody, produced by a cell line DSMZ Deposit No. DSM ACC 2600, or represented by heavy and light chain sequences set forth as SEQ ID NOs:1 and 2. The percent identity of such antibodies and functional fragments can be as little as 60%, or can be more (e.g., 65%, 70%, 75%, 80%, 85%, 90%, 95%, etc.).

The percent identity can extend over the entire sequence length of PM-2 antibody, produced by a cell line DSMZ Deposit No. DSM ACC 2600, or represented by heavy and light chain sequences set forth as SEQ ID NOs:1 and 2, or a contiguous region or area within PM-2 antibody, produced by a cell line DSMZ Deposit No. DSM ACC 2600, or represented by heavy and light chain sequences set forth as SEQ ID NOs:1 and 2. In particular aspects, the length of the sequence sharing the percent identity is 5 or more contiguous amino acids, e.g., 5, 6, 7, 8, 9, 10, 11, 12, 13, 14, 15, 16, 17, 18, 19, 20, 21, 22, 23, 24, 25, etc. contiguous amino acids. In additional particular aspects, the length of the sequence sharing the percent identity is 25 or more contiguous amino acids, e.g., 26, 27, 28, 29, 30, 31, 32, 33, 34, 35, etc. contiguous amino acids. In further particular aspects, the length of the sequence sharing the percent identity is 35 or more contiguous amino acids, e.g., 35, 36, 37, 38, 39, 40, 41, 42, 43, 44, 45, 45, 47, 48, 49, 50, etc., contiguous amino acids. In yet additional particular aspects, the length of the sequence sharing the percent identity is 50 or more contiguous amino acids, e.g., 50-55, 55-60, 60-65, 65-70, 70-75, 75-80, 80-85, 85-90, 90-95, 95-100, 100-110, etc. contiguous amino acids. In yet further particular aspects, the length of the sequence sharing the percent identity is equal to the length of any CDR of a variable region sequence (e.g., amino acids 11-18, 36-43, or 82-100 of SEQ ID NO:1, or amino acids 26-34, 52-58, or 97-103 of SEQ ID NO:2), or a region outside the CDRs but within the variable region of a heavy or light chain sequence, such as PM-2 antibody, produced by a cell line DSMZ Deposit No. DSM ACC 2600, or represented by heavy and light chain sequences set forth as SEQ ID NOs:1 and 2.

The term “identity” and grammatical variations thereof, mean that two or more referenced entities are the same. Thus, where two antibody sequences are identical, they have the same amino acid sequence, at least within the referenced region or portion. Where two nucleic acid sequences are identical, they have the same polynucleotide sequence, at least within the referenced region or portion. The identity can be over a defined area (region or domain) of the sequence. An “area of identity” refers to a portion of two or more referenced entities that are the same. Thus, where two protein or nucleic acid sequences are identical over one or more sequence regions they share identity within that region. Exemplary identity are antibodies and functional fragments with an amino acid sequence with 50%, 60%, 70%, 75%, 80%, 85%, 90%, 95%, 96%, 97%, 98%, or more sequence identity to a reference antibody or functional fragment, for example, PM-2 antibody, produced by a cell line DSMZ Deposit No. DSM ACC 2600, or represented by heavy and light chain sequences set forth as SEQ ID NOs:1 and 2, or a subsequence thereof.

The terms “homologous” or “homology” mean that two or more referenced entities share at least partial identity over a given region or portion. “Areas, regions or domains” of homology or identity mean that a portion of two or more referenced entities share homology or are the same. Thus, where two antibody sequences are identical over one or more sequence regions they share identity in these regions. “Substantial homology” means that a molecule is structurally or functionally conserved such that it has or is predicted to have at least partial structure or function of one or more of the structures or functions (e.g., a biological function) of the reference molecule, or relevant/corresponding region or portion of the reference molecule to which it shares homology. An antibody or functional fragment with substantial homology has or is predicted to have at least partial activity or function as the reference antibody. For example, in a particular embodiment, a PM-2 antibody, produced by a cell line DSMZ Deposit No. DSM ACC 2600, or represented by heavy and light chain sequences set forth as SEQ ID NOs:1 and 2, with one or more modifications (e.g., substitutions, deletions or additions of PM-2 antibody, produced by a cell line DSMZ Deposit No. DSM ACC 2600, or represented by heavy and light chain sequences set forth as SEQ ID NOs:1 and 2) retain the ability to at least partially compete for binding of PM-2 antibody, produced by a cell line DSMZ Deposit No. DSM ACC 2600, or represented by heavy and light chain sequences set forth as SEQ ID NOs:1 and 2, to a cell, antigen or epitope, or at least retains partial binding to a cell, antigen or epitope to which PM-2 antibody, produced by a cell line DSMZ Deposit No. DSM ACC 2600, or represented by heavy and light chain sequences set forth as SEQ ID NOs:1 and 2, binds is considered to have substantial homology to PM-2 antibody, produced by a cell line DSMZ Deposit No. DSM ACC 2600, or represented by heavy and light chain sequences set forth as SEQ ID NOs:1 and 2.

The extent of identity (homology) between two sequences can be ascertained using a computer program and mathematical algorithm known in the art. Such algorithms that calculate percent sequence identity (homology) generally account for sequence gaps and mismatches over the comparison region or area. For example, a BLAST (e.g., BLAST 2.0) search algorithm (see, e.g., Altschul et al., J. Mol. Biol. 215:403 (1990), publicly available through NCBI) has exemplary search parameters as follows: Mismatch −2; gap open 5; gap extension 2. For polypeptide sequence comparisons, a BLASTP algorithm is typically used in combination with a scoring matrix, such as PAM100, PAM 250, BLOSUM 62 or BLOSUM 50. FASTA (e.g., FASTA2 and FASTA3) and SSEARCH sequence comparison programs are also used to quantitate the extent of identity (Pearson et al., Proc. Natl. Acad. Sci. USA 85:2444 (1988); Pearson, Methods Mol. Biol. 132:185 (2000); and Smith et al., J. Mol. Biol. 147:195 (1981)). Programs for quantitating protein structural similarity using Delaunay-based topological mapping have also been developed (Bostick et al., Biochem Biophys Res Commun. 304:320 (2003)).

Antibodies and functional fragments of the invention include those that retain at least one or more partial activities or functions of PM-2 antibody, produced by a cell line DSMZ Deposit No. DSM ACC 2600, or represented by heavy and light chain sequences set forth as SEQ ID NOs:1 and 2. As disclosed herein, the antigen to which PM-2 antibody, produced by a cell line DSMZ Deposit No. DSM ACC 2600, or represented by heavy and light chain sequences set forth as SEQ ID NOs:1 and 2, binds is expressed on malignant and non-malignant, neoplastic, tumor and cancer cells. Non-limiting examples of cells that bind to PM-2 antibody, produced by a cell line DSMZ Deposit No. DSM ACC 2600, or represented by heavy and light chain sequences set forth as SEQ ID NOs:1 and 2, and therefore express a target antigen of PM-2 include a stomach adenocarcinoma, colorectal adenocarcinoma, squamous cell lung carcinoma, lung adenocarcinoma, squamous cell carcinoma of the esophagus, adenocarcinoma of the pancreas, urothel carcinoma of the urinary bladder, renal cell carcinoma of the kidney, adenocarcinoma of the prostate, ductal carcinoma of the breast, lobular carcinoma of the breast, adenocarcinoma of the ovary, adenocarcinoma of the adrenal gland, adenocarcinoma of the endometrium or a uterus cell adenocarcinoma, or HT-29 (ATCC Accession No. HTB-38; DSMZ Accession No. ACC 299), CACO-2 (ATCC Accession No. HBT-37; DSMZ Accession No. ACC 169), COLO-320 (DSMZ Accession No. ACC 144), COLO-206F (DSMZ Accession No. ACC 21), ASPC-1 (ATCC Accession No. CRL-1682), BXPC-3 (ATCC Accession No. CRL-1687) or A549 (ATCC Accession No. CCL-185) cells. Thus, in various embodiments, an antibody or functional fragment binds to one or more cells, such as a stomach adenocarcinoma, colorectal adenocarcinoma, squamous cell lung carcinoma, lung adenocarcinoma, squamous cell carcinoma of the esophagus, adenocarcinoma of the pancreas, urothel carcinoma of the urinary bladder, renal cell carcinoma of the kidney, adenocarcinoma of the prostate, ductal carcinoma of the breast, lobular carcinoma of the breast, adenocarcinoma of the ovary, adenocarcinoma of the adrenal gland, adenocarcinoma of the endometrium or a uterus cell adenocarcinoma, or HT-29 (ATCC Accession No. HTB-38; DSMZ Accession No. ACC 299), CACO-2 (ATCC Accession No. HBT-37; DSMZ Accession No. ACC 169), COLO-320 (DSMZ Accession No. ACC 144), COLO-206F (DSMZ Accession No. ACC 21), ASPC-1 (ATCC Accession No. CRL-1682), BXPC-3 (ATCC Accession No. CRL-1687) or A549 (ATCC Accession No. CCL-185) cells.

Antibodies and functional fragments that bind to a cell, antigen or epitope to which PM-2 antibody, produced by a cell line DSMZ Deposit No. DSM ACC 2600, or represented by heavy and light chain sequences set forth as SEQ ID NOs:1 and 2, binds (or competes for binding) can have greater or less relative binding affinity for a cell, antigen or epitope than PM-2 antibody, produced by a cell line DSMZ Deposit No. DSM ACC 2600, or represented by heavy and light chain sequences set forth as SEQ ID NOs:1 and 2. Additional antibodies and functional fragments of the invention therefore include those that have greater than, about the same or less than the binding affinity of PM-2 antibody, produced by a cell line DSMZ Deposit No. DSM ACC 2600, or represented by heavy and light chain sequences set forth as SEQ ID NOs:1 and 2, for binding to a cell, antigen or epitope. For example, an antibody or functional fragment of the invention may have an affinity greater or less than 2-5, 5-10, 10-100, 100-1000 or 1000-10,000-fold affinity, or any numerical value or range within or encompassing such values, than PM-2 antibody, produced by a cell line DSMZ Deposit No. DSM ACC 2600, or represented by heavy and light chain sequences set forth as SEQ ID NOs:1 and 2. In one embodiment, an antibody or a functional thereof has a binding affinity within about 1-5000 fold of the binding affinity of PM-2 antibody, produced by a cell line DSMZ Deposit No. DSM ACC 2600, or represented by heavy and light chain sequences set forth as SEQ ID NOs:1 and 2, for binding to a neoplastic, cancer, tumor or metastatic cell. In another embodiment, an antibody or a functional thereof has a binding affinity within about 1-5000 fold of the binding affinity of PM-2 antibody, produced by a cell line DSMZ Deposit No. DSM ACC 2600, or represented by heavy and light chain sequences set forth as SEQ ID NOs:1 and 2, for binding to a stomach adenocarcinoma, colorectal adenocarcinoma, squamous cell lung carcinoma, lung adenocarcinoma, squamous cell carcinoma of the esophagus, adenocarcinoma of the pancreas, urothel carcinoma of the urinary bladder, renal cell carcinoma of the kidney, adenocarcinoma of the prostate, ductal carcinoma of the breast, lobular carcinoma of the breast, adenocarcinoma of the ovary, adenocarcinoma of the adrenal gland, adenocarcinoma of the endometrium or a uterus cell adenocarcinoma. In a further embodiment, an antibody or a functional thereof has a binding affinity within about 1-5000 fold of the binding affinity of PM-2 antibody, produced by a cell line DSMZ Deposit No. DSM ACC 2600, or represented by heavy and light chain sequences set forth as SEQ ID NOs:1 and 2, for binding to HT-29 (ATCC Accession No. HTB-38; DSMZ Accession No. ACC 299), CACO-2 (ATCC Accession No. HBT-37; DSMZ Accession No. ACC 169), COLO-320 (DSMZ Accession No. ACC 144), COLO-206F (DSMZ Accession No. ACC 21), ASPC-1 (ATCC Accession No. CRL-1682), BXPC-3 (ATCC Accession No. CRL-1687) or A549 (ATCC Accession No. CCL-185) cells. In the foregoing embodiments binding affinity can be 1-5000 fold greater or less than the binding affinity of PM-2 antibody, produced by a cell line DSMZ Deposit No. DSM ACC 2600, or represented by heavy and light chain sequences set forth as SEQ ID NOs:1 and 2.

Binding affinity can be determined by association (Kₐ) and dissociation (K_{d}) rate. Equilibrium affinity constant, K, is the ratio of Kₐ/K_{d}. Association (Kₐ) and dissociation (K_{d}) rates can be measured using surface plasmon resonance (SPR) (Rich and Myszka, Curr. Opin. Biotechnol. 11:54 (2000); Englebienne, Analyst 123:1599 (1998)). Instrumentation and methods for real time detection and monitoring of binding rates are known and are commercially available (BiaCore 2000, Biacore AB, Upsala, Sweden; and Malmqvist, Biochem. Soc. Trans. 27:335 (1999)).

Additional specific non-limiting antibodies and functional fragments have binding affinity for (or compete for binding to) a cell, antigen or epitope to which PM-2 antibody, produced by a cell line DSMZ Deposit No. DSM ACC 2600, or represented by heavy and light chain sequences set forth as SEQ ID NOs:1 and 2, within about K_{d} 10⁻² M to about K_{d} 10⁻¹⁵ M, or within about K_{d} 10⁻⁶ M to about K_{d} 10⁻¹² M. In particular embodiments, binding affinity for is less than 5×10⁻² M, 10⁻² M, 5×10⁻³ M, 10⁻³ M 5×10⁻⁴ M, 10⁻⁴ M 5×10⁻⁵ M, 10⁻⁵ M 5×10⁻⁶ M, 10⁻⁶ M 5×10⁻⁷ M, 10⁻⁷ M 5×10⁻⁸ M, 10⁻⁸ M 5×10⁻⁹ M, 10⁻⁹ M 5×10⁻¹⁰ M, 10⁻¹⁰ M 5×10⁻¹¹ M, 10⁻¹¹ M 5×10⁻¹² M, 10⁻¹² M 5×10⁻¹³ M, 10⁻¹³ M 5×10⁻¹⁴ M, 10⁻¹⁴ M 5×10⁻¹⁵ M, and 10⁻¹⁵ M. In particular embodiments, an antibody or functional fragment has a binding affinity within about K_{d} 10⁻⁵ M to about K_{d} 10⁻¹³ M for binding to a neoplastic, cancer, tumor or metastatic cell. In additional particular embodiments, an antibody or functional fragment has a binding affinity within about K_{d} 10⁻⁵ M to about K_{d} 10⁻¹³ M for binding to an adenocarcinoma cell or a squamous cell carcinoma, such as a stomach adenocarcinoma, colorectal adenocarcinoma, squamous cell lung carcinoma, lung adenocarcinoma, squamous cell carcinoma of the esophagus, adenocarcinoma of the pancreas, urothel carcinoma of the urinary bladder, renal cell carcinoma of the kidney, adenocarcinoma of the prostate, ductal carcinoma of the breast, lobular carcinoma of the breast, adenocarcinoma of the ovary, adenocarcinoma of the adrenal gland, adenocarcinoma of the endometrium or a uterus cell adenocarcinoma. In further particular embodiments, an antibody or functional fragment has a binding affinity within about K_{d} 10⁻⁵ M to about K_{d} 10⁻¹³ M for binding to HT-29 (ATCC Accession No. HTB-38; DSMZ Accession No. ACC 299), CACO-2 (ATCC Accession No. HBT-37; DSMZ Accession No. ACC 169), COLO-320 (DSMZ Accession No. ACC 144), COLO-206F (DSMZ Accession No. ACC 21), ASPC-1 (ATCC Accession No. CRL-1682), BXPC-3 (ATCC Accession No. CRL-1687) or A549 (ATCC Accession No. CCL-185) cells.

Antibodies and functional fragments that bind to a cell, antigen or epitope to which PM-2 antibody, produced by a cell line DSMZ Deposit No. DSM ACC 2600, or represented by heavy and light chain sequences set forth as SEQ ID NOs:1 and 2, binds, or that compete with PM-2 antibody, produced by a cell line DSMZ Deposit No. DSM ACC 2600, or represented by heavy and light chain sequences set forth as SEQ ID NOs:1 and 2, for binding to a cell, antigen or epitope can have greater or less relative cell proliferation inhibiting or reducing activity, or greater or less relative cell apoptosis inducing or stimulating activity than PM-2 antibody, produced by a cell line DSMZ Deposit No. DSM ACC 2600, or represented by heavy and light chain sequences set forth as SEQ ID NOs:1 and 2. Antibodies and functional fragments of the invention therefore include those that bind to a cell, antigen or epitope to which PM-2 antibody, or compete with PM-2 antibody for binding to a cell, antigen or epitope, and have greater or less relative cell proliferation inhibiting or reducing activity, or greater or less relative cell apoptosis inducing or stimulating activity than PM-2 antibody, produced by a cell line DSMZ Deposit No. DSM ACC 2600, or represented by heavy and light chain sequences set forth as SEQ ID NOs:1 and 2.

Invention antibodies therefore include those that have a sequence distinct from PM-2 antibody, produced by a cell line DSMZ Deposit No. DSM ACC 2600, or represented by heavy and light chain sequences set forth as SEQ ID NOs:1 and 2, but that retain one or more activities or functions, at least in part, of PM-2 antibody, produced by a cell line DSMZ Deposit No. DSM ACC 2600, or represented by heavy and light chain sequences set forth as SEQ ID NOs:1 and 2. Exemplary activities and functions include, for example, binding to a cell to which PM-2 antibody binds; binding to an antigen to which PM-2 antibody binds; binding to an epitope to which PM-2 antibody binds; competing with PM-2 antibody for binding to a cell, antigen or an epitope; inhibiting or reducing cell growth or proliferation, or stimulating or inducing cell death, lysis or apoptosis (e.g., a neoplastic, tumor or cancer, or metastasis cell); binding to one or more of a stomach adenocarcinoma, colorectal adenocarcinoma, squamous cell lung carcinoma, lung adenocarcinoma, squamous cell carcinoma of the esophagus, adenocarcinoma of the pancreas, urothel carcinoma of the urinary bladder, renal cell carcinoma of the kidney, adenocarcinoma of the prostate, ductal carcinoma of the breast, lobular carcinoma of the breast, adenocarcinoma of the ovary, adenocarcinoma of the adrenal gland, adenocarcinoma of the endometrium or a uterus cell adenocarcinoma; inhibiting HT-29 (ATCC Accession No. HTB-38; DSMZ Accession No. ACC 299), CACO-2 (ATCC Accession No. HBT-37; DSMZ Accession No. ACC 169), COLO-320 (DSMZ Accession No. ACC 144), COLO-206F (DSMZ Accession No. ACC 21), ASPC-1 (ATCC Accession No. CRL-1682), BXPC-3 (ATCC Accession No. CRL-1687) or A549 (ATCC Accession No. CCL-185) cell growth or proliferation, or stimulating or inducing HT-29 (ATCC Accession No. HTB-38; DSMZ Accession No. ACC 299), CACO-2 (ATCC Accession No. HBT-37; DSMZ Accession No. ACC 169), COLO-320 (DSMZ Accession No. ACC 144), COLO-206F (DSMZ Accession No. ACC 21), ASPC-1 (ATCC Accession No. CRL-1682), BXPC-3 (ATCC Accession No. CRL-1687) or A549 (ATCC Accession No. CCL-185) cell death, lysis or apoptosis, etc.

Thus, in accordance with the invention there are also provided modified antibodies and functional fragments provided that the modified form retains, at least a part of an activity or function of unmodified or reference antibody, or functional fragment. In one embodiment, an antibody or a functional fragment thereof includes a heavy or a light chain variable region sequence with one or more amino acid additions, deletions or substitutions of PM-2 antibody, produced by a cell line DSMZ Deposit No. DSM ACC 2600, or represented by heavy and light chain sequences set forth as SEQ ID NOs:1 and 2, provided said antibody or functional fragment retains at least partial activity or function of intact full length PM-2 antibody, produced by a cell line DSMZ Deposit No. DSM ACC 2600, or represented by heavy and light chain sequences set forth as SEQ ID NOs:1 and 2. In one aspect, an antibody or a functional fragment with one or more amino acid additions, deletions or substitutions of PM-2 antibody, produced by a cell line DSMZ Deposit No. DSM ACC 2600, or represented by heavy and light chain sequences set forth as SEQ ID NOs:1 and 2, competes for binding to a cell, antigen or epitope to which PM-2 antibody, produced by a cell line DSMZ Deposit No. DSM ACC 2600, or represented by heavy and light chain sequences set forth as SEQ ID NOs:1 and 2, binds. In another aspect, an antibody or a functional fragment with one or more amino acid deletions, substitutions or additions of PM-2 antibody, produced by a cell line DSMZ Deposit No. DSM ACC 2600, or represented by heavy and light chain sequences set forth as SEQ ID NOs:1 and 2, binds to a cell, antigen or epitope to which PM-2 antibody binds. In an additional aspect, an antibody or a functional fragment with one or more amino acid deletions, substitutions or additions of PM-2 antibody, produced by a cell line DSMZ Deposit No. DSM ACC 2600, or represented by heavy and light chain sequences set forth as SEQ ID NOs:1 and 2, inhibits or reduces proliferation of a cell in which PM-2 antibody inhibits or reduces proliferation. In a further aspect, an antibody or a functional fragment with one or more amino acid deletions, substitutions or additions of PM-2 antibody, produced by a cell line DSMZ Deposit No. DSM ACC 2600, or represented by heavy and light chain sequences set forth as SEQ ID NOs:1 and 2, stimulates or induces death, lysis or apoptosis of a cell in which PM-2 antibody stimulates or induces death, lysis or apoptosis. In still further particular aspects, cell growth or proliferation is inhibited, decreased or reduced at least 20%, 30%, 40%, 50%, 60%, 75%, or more relative to a control (untreated) cell, or any numerical value or range within or encompassing such percent values. In yet further particular aspects, cell death, lysis or apoptosis is at least 20%, 30%, 40%, 50%, 60%, 75%, or more relative to a control (untreated) cell, or any numerical value or range within or encompassing such percent values.

As used herein, the term “modify” and grammatical variations thereof, means that the composition deviates from a reference composition. Such modified proteins, nucleic acids and other compositions may have greater or less activity than or a distinct function from a reference unmodified protein, nucleic acid, or composition.

Modifications, which include substitutions, additions and deletions, can also be referred to as “variants.” Specific non-limiting examples of amino acid variants include PM-2 antibody, produced by a cell line DSMZ Deposit No. DSM ACC 2600, or represented by heavy and light chain sequences set forth as SEQ ID NOs:1 and 2, fragments and subsequences. Exemplary PM-2 antibody subsequences and fragments include a portion of the PM-2 antibody, produced by a cell line DSMZ Deposit No. DSM ACC 2600, or represented by heavy and light chain sequences set forth as SEQ ID NOs:1 and 2, that at least partially competes with PM-2 antibody for binding to a cell, antigen or epitope, or that retains at least partial binding activity to a cell, antigen or epitope to which PM-2 antibody, produced by a cell line DSMZ Deposit No. DSM ACC 2600, or represented by heavy and light chain sequences set forth as SEQ ID NOs:1 and 2, binds, or that retains an ability to inhibit or reduce proliferation of a cell in which PM-2 antibody inhibits or reduces proliferation, or that retains an ability to stimulate or induce death, lysis or apoptosis of a cell in which PM-2 antibody stimulates or induces death, lysis or apoptosis.

As used herein, the term “fragment” or “subsequence” means a portion of the full length molecule. Thus, a fragment or subsequence of an antibody has one or more less amino acids than a full length intact reference antibody (e.g. one or more internal or terminal amino acid deletions from either amino or carboxy-termini of heavy or light chain variable or constant regions). A nucleic acid fragment has at least one less nucleotide than a full length comparison nucleic acid sequence. Fragments therefore can be any length up to the full length native molecule.

The terms “functional fragment” and “functional subsequence” when referring to an antibody refers to a portion of an antibody with a function or activity. For example, a functional fragment can retain one or more partial functions or activities as an intact reference antibody, e.g., a function or activity of PM-2 antibody, produced by a cell line DSMZ Deposit No. DSM ACC 2600, or represented by heavy and light chain sequences set forth as SEQ ID NOs:1 and 2. For example, a PM-2 antibody subsequence that competes for binding of full length intact PM-2 antibody, produced by a cell line DSMZ Deposit No. DSM ACC 2600, or represented by heavy and light chain sequences set forth as SEQ ID NOs:1 and 2, to a cell or to an antigen, or that binds to a cell or antigen to which full length intact PM-2 antibody binds is considered a functional subsequence.

Antibody fragments, including single-chain antibodies, can include all or a portion of heavy or light chain variable region(s) (e.g., one or more CDRs, such as CDR1, CDR2 or CDR3, respectively amino acids 11-18, 36-43, or 82-100 of SEQ ID NO:1, and amino acids 26-34, 52-58, or 97-103 of SEQ ID NO:2) alone or in combination with all or a portion of one or more of the following: hinge region, CH1, CH2, and CH3 domains. Also included are antigen-binding subsequences of any combination of heavy or light chain variable region(s) (e.g., one or more CDRs, such as CDR1, CDR2 or CDR3, respectively amino acids 11-18, 36-43, or 82-100 of SEQ ID NO:1, and amino acids 26-34, 52-58, or 97-103 of SEQ ID NO:2) with a hinge region, CH1, CH2, and CH3 domains.

Exemplary antibody subsequences and fragments of the invention include Fab, Fab′, F(ab′)₂, Fv, Fd, single-chain Fv (scFv), disulfide-linked Fvs (sdFv), V_{L}, V_{H}, trispecific (Fab₃), bispecific (Fab₂), diabody ((V_{L}-V_{H})₂ or (V_{H}-V_{L})₂), triabody (trivalent), tetrabody (tetravalent), minibody ((scF_{V}-C_{H}3)₂), bispecific single-chain Fv (Bis-scFv), IgGdeltaCH2, scFv-Fc and (scFv)₂-Fc. Such subsequences and fragments can have binding affinity as the full length antibody, the binding specificity as the full length antibody, or one or more activities or functions of as a full length antibody, e.g., a function or activity of PM-2 antibody, produced by a cell line DSMZ Deposit No. DSM ACC 2600, or represented by heavy and light chain sequences set forth as SEQ ID NOs:1 and 2.

Antibody subsequences and fragments can be combined. For example, a V_{L or V}_{H} subsequences can be joined by a linker sequence thereby forming a V_{L}-V_{H} chimera. In particular, a heavy chain variable sequence of PM-2 antibody, produced by a cell line DSMZ Deposit No. DSM ACC 2600, or represented by heavy and light chain sequences set forth as SEQ ID NOs:1 and 2, can be combined with a light chain variable sequence of PM-2 antibody, produced by a cell line DSMZ Deposit No. DSM ACC 2600, or represented by heavy and light chain sequences set forth as SEQ ID NOs:1 and 2. The invention therefore provides: 1) heavy chain variable sequence of PM-2 antibody, produced by a cell line DSMZ Deposit No. DSM ACC 2600, or represented by heavy chain variable sequence set forth as SEQ ID NO:1; and 2) light chain variable sequence of PM-2 antibody, produced by a cell line DSMZ Deposit No. DSM ACC 2600, or represented by light chain variable sequence set forth as SEQ ID NO:2 alone and in combination with each other. A combination of single-chain Fvs (scFv) subsequences can be joined by a linker sequence thereby forming a scFv-scFv chimera. Antibody subsequences and fragments include single-chain antibodies or variable region(s) alone or in combination with all or a portion of other subsequences.

Modified proteins further include amino acid substitutions. Substitutions can be conservative or non-conservative and may be in a constant or variable (e.g., hypervariable, such as CDR or FR) region of PM-2 antibody, produced by a cell line DSMZ Deposit No. DSM ACC 2600, or represented by heavy and light chain sequences set forth as SEQ ID NOs:1 and 2. In particular embodiments, a modified PM-2 antibody, produced by a cell line DSMZ Deposit No. DSM ACC 2600, or represented by heavy and light chain sequences set forth as SEQ ID NOs:1 and 2, has one or a few conservative or non-conservative amino acid substitutions.

Antibody structural determinants that contribute to antigen binding, such as complementarity determining regions (CDR, of which there are three in each heavy and light chain sequence, conveniently denoted as HC-CDR1, HC-CDR2 and HC-CDR3; and LC-CDR1, LC-CDR2 and LC-CDR3; respectively amino acids 11-18, 36-43, and 82-100 of SEQ ID NO:1, and amino acids 26-34, 52-58, and 97-103 of SEQ ID NO:2) within hypervariable regions are known to the skilled artisan. The location of additional regions, such as D- and J-regions are also known to the skilled artisan. Antibodies and subsequences thereof in which one or more CDR sequences have sufficient sequence identity to PM-2 antibody, produced by a cell line DSMZ Deposit No. DSM ACC 2600, or represented by heavy and light chain sequences set forth as SEQ ID NOs:1 and 2, so as to retain at least partial function or activity of PM-2 antibody, produced by a cell line DSMZ Deposit No. DSM ACC 2600, or represented by heavy and light chain sequences set forth as SEQ ID NOs:1 and 2, e.g., cell, antigen or epitope binding, binding affinity (e.g., K_{d}), cell proliferation inhibition, or stimulating or inducing cell apoptosis, etc.

Accordingly, amino acid substitutions in constant or variable regions of PM-2 antibody, produced by a cell line DSMZ Deposit No. DSM ACC 2600, or represented by heavy and light chain sequences set forth as SEQ ID NOs:1 and 2, are likely to be tolerated. One or a few substitutions in a variable region outside of a CDR of PM-2 antibody, produced by a cell line DSMZ Deposit No. DSM ACC 2600, or represented by heavy and light chain sequences set forth as SEQ ID NOs:1 and 2, is also likely to be tolerated at least to the extent that at least partial cell, antigen or epitope binding activity is retained, or partial cell proliferation inhibiting or apoptosis stimulating or inducing activity is retained. One or a few conservative substitutions in a CDR of PM-2 antibody, produced by a cell line DSMZ Deposit No. DSM ACC 2600, or represented by heavy and light chain sequences set forth as SEQ ID NOs:1 and 2 (e.g., amino acids 11-18, 36-43, or 82-100 of SEQ ID NO:1, and amino acids 26-34, 52-58, or 97-103 of SEQ ID NO:2), is also likely to be tolerated at least to the extent that at least partial cell, antigen or epitope binding activity is retained (i.e., cell or antigen binding is not destroyed), or partial cell proliferation inhibiting or apoptosis stimulating or inducing activity is retained. Non-conservative substitution of many amino acids in hypervariable regions (e.g., CDRs) of PM-2 antibody, produced by a cell line DSMZ Deposit No. DSM ACC 2600, or represented by heavy and light chain sequences set forth as SEQ ID NOs:1 and 2, is likely to affect one or more of cell, antigen or epitope binding activity, binding affinity (e.g., K_{d}), or antibody function or activity, such as cell proliferation inhibition, stimulating or inducing cell apoptosis, etc.

A “conservative substitution” is the replacement of one amino acid by a biologically, chemically or structurally similar residue. Biologically similar means that the substitution does not destroy a biological activity, e.g., cell binding or cell proliferation inhibiting or apoptosis inducing or stimulating activity. Structurally similar means that the amino acids have side chains with similar length, such as alanine, glycine and serine, or a similar size. Chemical similarity means that the residues have the same charge or are both hydrophilic or hydrophobic. Particular examples include the substitution of one hydrophobic residue, such as isoleucine, valine, leucine or methionine for another, or the substitution of one polar residue for another, such as the substitution of arginine for lysine, glutamic for aspartic acids, or glutamine for asparagine, serine for threonine, and the like.

In particular embodiments, a heavy or light chain hypervariable region sequence or a region therein, such as a CDR (CDR1, CDR2 or CDR3; amino acids 11-18, 36-43, or 82-100 of SEQ ID NO:1, or amino acids 26-34, 52-58, or 97-103 of SEQ ID NO:2) or FR will have 1-10, 1-5, 1-3 or fewer (e.g., 1 or 2) amino acid substitutions. In an additional embodiment, an amino acid substitution within a heavy or light chain hypervariable region sequence is not within more than one CDR. In an additional embodiment, a substitution within a heavy or light chain hypervariable region sequence is not within a CDR. In another embodiment, a substitution within a hypervariable region sequence is not within an FR.

The effect of a given modification can be readily assayed in order to identify antibodies and functional fragments retaining at least a part of the cell or antigen binding activity, affinity or antibody function or activity of unmodified antibody, e.g., PM-2 antibody, produced by a cell line DSMZ Deposit No. DSM ACC 2600, or represented by heavy and light chain sequences set forth as SEQ ID NOs:1 and 2. For example, an amino acid substitution in a variable region (e.g., within or outside of CDR1, CDR2 or CDR3) of PM-2 antibody, produced by a cell line DSMZ Deposit No. DSM ACC 2600, or represented by heavy and light chain sequences set forth as SEQ ID NOs:1 and 2, can be assayed for cell. antigen or epitope binding, cell proliferation inhibiting or reducing activity, inducing or stimulating cell death, lysis or apoptosis, etc.

Regional mutability analysis can be used to predict the effect of particular substitutions in complementarity determining regions (CDR) and framework regions (FR) (Shapiro et al., J. Immunol. 163:259 (1999)). In brief, sequence comparison indicates a hierarchy of mutability among di- and trinucleotide sequences located within Ig intronic DNA, which predicts regions that are more or less mutable. Quantitative structure-activity relationship (QSAR) can be used to identify the nature of the antibody recognition domain and, therefore, amino acids that participate in ligand binding. Predictive models based upon OSAR can in turn be used to predict the effect of substitutions (mutations). For example, the effect of mutations on the association and dissociation rate of an antibody interacting with its antigen has been used to construct quantitative predictive models for both kinetic (Kₐ and K_{d}) constants, which in turn is used to predict the effect of other mutations on the antibody (De Genst et al., J Biol. Chem. 277:29897 (2002)). The skilled artisan can therefore use such analysis to identify amino acid substitutions of antibodies and functional fragments that are likely to result in an antibody or functional fragment that retains at least partial activity or function of non-substituted antibody or functional fragment.

Another method for identifying residues or regions for mutagenesis is called “alanine scanning mutagenesis” which is described, for example, by Cunningham and Wells (Science 244:1081 (1989)). A residue or group of target residues are identified (e.g., charged residues such as arg, asp, his, lys, and glu) and replaced by a neutral or negatively charged amino acid (most desirably alanine or polyalanine) to affect the interaction of the amino acids with the surrounding aqueous environment in or outside the cell. The domains demonstrating functional sensitivity to the substitutions then are refined by introducing further or other variants at or for the sites of substitution. Thus, while the site for introducing an amino acid sequence variation is predetermined, the mutation need not be predetermined. For instance, to optimize the performance of a mutation at a given site, alanine scanning or random mutagenesis may be conducted at the target codon or region and the expressed variants are screened for antigen or cell binding, or the ability to induce apoptosis or inhibit proliferation of a neoplastic, tumor, cancer or metastatic cell.

Amino acid substitutions may be with the same amino acid, except that a naturally occurring L-amino acid is substituted with a D-form amino acid. Modifications therefore include one or more D-amino acids substituted for L-amino acids, or mixtures of D-amino acids substituted for L-amino acids. Modifications also include structural and functional analogues, for example, peptidomimetics having synthetic or non-natural amino acids or amino acid analogues and derivatized forms.

Modified forms further include derivatized sequences, for example, amino acids in which free amino groups form amine hydrochlorides, p-toluene sulfonyl groups, carbobenzoxy groups; the free carboxy groups from salts, methyl and ethyl esters; free hydroxyl groups that form O-acyl or O-alkyl derivatives, as well as naturally occurring amino acid derivatives, for example, 4-hydroxyproline, for proline, 5-hydroxylysine for lysine, homoserine for serine, ornithine for lysine, etc. Modifications can be produced using methods known in the art (e.g., PCR based site-directed, deletion and insertion mutagenesis, chemical modification and mutagenesis, cross-linking, etc.).

Modified forms include additions and insertions. For example, an addition can be the covalent or non-covalent attachment of any type of molecule to a protein (e.g., antibody), nucleic acid or other composition. Typically additions and insertions confer a distinct function or activity.

Additions and insertions include fusion (chimeric) polypeptide or nucleic acid sequences, which is a sequence having one or more molecules not normally present in a reference native (wild type) sequence covalently attached to the sequence. A particular example is an amino acid sequence of another protein (e.g., antibody) to produce a multifunctional protein (e.g., multispecific antibody).

In accordance with the invention, there are provided antibodies, nucleic acids, and other compositions that include a heterologous domain. Thus, a heterologous domain can consist of any of a variety of different types of small or large functional moieties. Such moieties include nucleic acid, peptide, carbohydrate, lipid or small organic compounds, such as a drug (e.g., a cell anti-proliferative agent), metals (gold, silver), etc. A heterologous domain can be an amino acid addition or insertion.

Particular non-limiting examples of heterologous domains include, for example, tags, detectable labels and cytotoxic agents. Specific examples of tags and detectable labels include enzymes (horseradish peroxidase, urease, catalase, alkaline phosphatase, beta-galactosidase, chloramphenicol transferase); enzyme substrates; ligands (e.g., biotin); receptors (avidin); radionuclides (e.g., C¹⁴, S³⁵, P³², P³³, H³, I¹²⁵, I¹³¹, gallium-67 and 68, scantium-47, indium-111, radium-223); T7-, His-, myc-, HA- and FLAG-tags; electron-dense reagents; energy transfer molecules; paramagnetic labels; fluorophores (fluorescein, fluorescamine, rhodamine, phycoerythrin, phycocyanin, allophycocyanin); chromophores; chemi-luminescent (imidazole, luciferase, acridinium, oxalate); and bio-luminescent agents. Specific examples of cytotoxic agents (cytotoxins) include diphtheria, toxin, cholera toxin and ricin.

Additional examples of heterologous domains include, for example, anti-cell proliferative agents (e.g., anti-neoplastic, anti-tumor or anti-cancer, or anti-metastasis agents). Specific non-limiting examples of anti-cell proliferative agents (e.g., anti-neoplastic, anti-tumor or anti-cancer, or anti-metastasis agents, cytotoxins, etc.) are disclosed herein and known in the art.

Linker sequences may be inserted between the protein (e.g., antibody), nucleic acid, or other composition and the addition or insertion (e.g., heterologous domain) so that the two entities maintain, at least in part, a distinct function or activity. Linker sequences may have one or more properties that include a flexible structure, an inability to form an ordered secondary structure or a hydrophobic or charged character which could promote or interact with either domain. Amino acids typically found in flexible protein regions include Gly, Asn and Ser. Other near neutral amino acids, such as Thr and Ala, may also be used in the linker sequence. The length of the linker sequence may vary (see, e.g., U.S. Pat. No. 6,087,329). Linkers further include chemical cross-linking and conjugating agents, such as sulfo-succinimidyl derivatives (sulfo-SMCC, sulfo-SMPB), disuccinimidyl suberate (DSS), disuccinimidyl glutarate (DSG) and disuccinimidyl tartrate (DST).

Further examples of additions include glycosylation, fatty acids, lipids, acetylation, phosphorylation, amidation, formylation, ubiquitination, and derivatization by protecting/blocking groups and any of numerous chemical modifications. Other permutations and possibilities will be readily apparent to those of ordinary skill in the art, and are considered to be within the scope of the invention.

The term “isolated” used as a modifier of a composition means that the composition is made by the hand of man or is separated from one or more other components in their naturally occurring in vivo environment. Generally, compositions so separated are substantially free of one or more materials with which they normally associate with in nature, for example, one or more protein, nucleic acid, lipid, carbohydrate, cell membrane. Thus, an isolated composition is substantially separated from other biological components in the cell of the organism in which the composition naturally occurs, or from the artificial medium in which it is produced (e.g., synthetically or through cell culture). For example, an isolated polypeptide is substantially separated from other polypeptides and nucleic acid and does not include a library of polypeptides or polynucleotides present among millions of polypeptide or nucleic acid sequences, such as a polypeptide, genomic or cDNA library, for example. An isolated nucleic acid is substantially separated from other polypeptides and nucleic acid and does not include a library of polypeptides or polynucleotides present among millions of polypeptide or nucleic acid sequences, such as a polypeptide, genomic or cDNA library, for example. The term “isolated” does not exclude alternative physical forms of the composition, for example, an isolated protein could include protein multimers, post-translational modifications (e.g., glycosylation, phosphorylation) or derivatized forms.

The term “purified” used as a modifier of a composition refers to a composition free of most or all of the materials with which it typically associates with in nature. Thus, a protein separated from cells is considered to be substantially purified when separated from cellular components by standard methods while a chemically synthesized nucleic acid sequence is considered to be substantially purified when separated from its chemical precursors. Purified therefore does not require absolute purity. Furthermore, a “purified” composition can be combined with one or more other molecules. Thus, the term “purified” does not exclude combinations of compositions.

“Purified” proteins and nucleic acid include proteins and nucleic acids produced by standard purification methods. The term also includes proteins and nucleic acids produced by recombinant expression in a host cell as well as chemical synthesis. “Purified” can also refer to a composition in which the level of contaminants is below a level that is acceptable to a regulatory agency for administration to a human or non-human animal, for example, the Food and Drug administration (FDA).

Substantial purity can be at least about 60% or more of the molecule by mass. Purity can also be about 70% or 80% or more, and can be greater, for example, 90% or more. Purity can be less, for example, in a pharmaceutical carrier the amount of a molecule by weight % can be less than 60% but the relative proportion of the molecule compared to other components with which it is normally associated with will be greater. Purity can be determined by any appropriate method, including, for example, UV spectroscopy, chromatography (e.g., HPLC, gas phase), gel electrophoresis (e.g., silver or coomassie staining) and sequence analysis (peptide and nucleic acid).

Methods of producing polyclonal and monoclonal antibodies are known in the art. For example, PM-2 antigen or an immunogenic fragment thereof, optionally conjugated to a carrier such as keyhole limpet hemocyanin (KLH) or ovalbumin (e.g., BSA), or mixed with an adjuvant such as Freund's complete or incomplete adjuvant, and used to immunize an animal. Using conventional hybridoma technology, splenocytes from immunized animals that respond to PM-2 antigen can be isolated and fused with myeloma cells. Monoclonal antibodies produced by the hybridomas can be screened for reactivity with PM-2 antigen, for example, via ELISA. Additional non-limiting particular methods of antibody and functional fragment screening and selection include phage display, protein-mRNA link via ribosome and mRNA display, display on yeast, bacteria, mammalian cells or retroviruses, microbead via in vitro compartmentalization, protein-DNA display, growth selection via yeast 2-hybrid, protein fragment complementation (Hoogenboom, R., Nature Biotechnol. 23:1105 (2005)).

Antibodies that compete with PM-2 antibody, produced by a cell line DSMZ Deposit No. DSM ACC 2600, or represented by heavy and light chain sequences set forth as SEQ ID NOs:1 and 2, for binding to a cell, antigen or epitope can be screened and identified using a conventional competition binding assays. Screened antibodies are selected based upon an ability to compete with PM-2 antibody, produced by a cell line DSMZ Deposit No. DSM ACC 2600, or represented by heavy and light chain sequences set forth as SEQ ID NOs:1 and 2, for binding to a cell, antigen or epitope. The ability of an antibody to compete with PM-2 antibody for binding to a cell or antigen, or to inhibit, prevent or block binding of PM-2 antibody to a cell, antigen or epitope, can be determined by various assays know in the art, including enzyme linked immunosorbent assay (ELISA).

Proteins and antibodies, subsequences and fragments thereof, as well as other modified sequences can be produced by genetic methodology. Such techniques include expression of all or a part of the gene encoding the protein or antibody into a host cell such as Cos cells or E. coli. Such host cells can express full length or a fragment, for example, an scFv (see, e.g., Whitlow et al., In: Methods: A Companion to Methods in Enzymology 2:97 (1991), Bird et al., Science 242:423 (1988); and U.S. Pat. No. 4,946,778). Antibodies and functional fragments, and nucleic acid sequences can also be produced by chemical synthesis using methods known to the skilled artisan, for example, an automated peptide synthesis apparatus (see, e.g., Applied Biosystems, Foster City, Calif.).

Cells or antigen suitable for generating antibodies can be produced by any of a variety of standard protein purification or recombinant expression techniques known in the art. For example, PM-2 antigen is present on cells, such as HT-29 (ATCC Accession No. HTB-38; DSMZ Accession No. ACC 299), CACO-2 (ATCC Accession No. HBT-37; DSMZ Accession No. ACC 169), COLO-320 (DSMZ Accession No. ACC 144), COLO-206F (DSMZ Accession No. ACC 21), ASPC-1 (ATCC Accession No. CRL-1682), BXPC-3 (ATCC Accession No. CRL-1687), A549 (ATCC Accession No. CCL-185) MKN and CRL cells. Accordingly, whole cells, or preparations, cell extracts or fractions of such cells can be used to immunize animals in order to produce antibodies that compete with PM-2 antibody, produced by a cell line DSMZ Deposit No. DSM ACC 2600, or represented by heavy and light chain sequences set forth as SEQ ID NOs:1 and 2, for binding of to a cell or antigen, or that bind to a cell or antigen to which PM-2 antibody, produced by a cell line DSMZ Deposit No. DSM ACC 2600, or represented by heavy and light chain sequences set forth as SEQ ID NOs:1 and 2, binds, for example.

Animals that may be immunized include mice, rats, rabbits, goats, sheep, cows or steer, guinea pigs or primates. Initial and any optional subsequent immunization may be through intravenous, intraperitoneal, intramuscular, or subcutaneous routes. Subsequent immunizations may be at the same or at different concentrations of PM-2 antigen preparation, and may be at regular or irregular intervals.

Animals include those genetically modified to include human IgG gene loci, which can therefore be used to produce human antibodies. Transgenic animals with one or more human immunoglobulin genes that do not express endogenous immunoglobulins are described, for example in, U.S. Pat. No. 5,939,598. Additional methods for producing human polyclonal antibodies and human monoclonal antibodies are described (see, e.g., Kuroiwa et al., Nat. Biotechnol. 20:889 (2002); WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; and 5,939,598). An overview of the technology for producing human antibodies is described in Lonberg and Huszar (Int. Rev. Immunol. 13:65 (1995)).

Antibodies can also be generated using other techniques including hybridoma, recombinant, and phage display technologies, or a combination thereof (see U.S. Pat. Nos. 4,902,614, 4,543,439, and 4,411,993; see, also Monoclonal Antibodies, Hybridomas: A New Dimension in Biological Analyses, Plenum Press, Kennett, McKearn, and Bechtol (eds.), 1980, and Harlow et al., Antibodies: A Laboratory Manual, Cold Spring Harbor Laboratory Press, 2nd ed. 1988).

Antibody subsequences and fragments can be prepared by proteolytic hydrolysis of the antibody, for example, by pepsin or papain digestion of whole antibodies. Antibody subsequences and fragments produced by enzymatic cleavage with pepsin provide a 5S fragment denoted F(ab′)₂. This fragment can be further cleaved using a thiol reducing agent to produce 3.5S Fab′ monovalent fragments. Alternatively, an enzymatic cleavage using pepsin produces two monovalent Fab′ fragments and the Fc fragment directly (see, e.g., U.S. Pat. Nos. 4,036,945 and 4,331,647; and Edelman et al., Methods Enzymol. 1:422 (1967)). Single-chain Fvs and antibodies can be produced as described in U.S. Pat. Nos. 4,946,778 and 5,258,498; Huston et al., Methods Enzymol. 203:46 (1991); Shu et al., Proc. Natl. Acad. Sci. USA 90:7995 (1993); and Skerra et al., Science 240:1038 (1988). Other methods of cleaving antibodies, such as separation of heavy chains to form monovalent light-heavy chain fragments, further cleavage of fragments, or other enzymatic or chemical may also be used.

Modified antibodies and functional fragments having altered characteristics, such as increased binding affinity, can be produced using methods known to the skilled artisan art. For example, affinity maturation techniques can be used to improve antibody binding affinity (US 2004/0162413 A1; U.S. Pat. Nos. 6,656,467, 6,531,580, 6,590,079 and 5,955,358; Fiedler et al., Protein Eng. 15:931 (2002); Pancook et al., Hybrid. Hybridomics 20:383 (2001); Daugherty et al., Protein Eng. 11:825 (1998); Wu et al., Proc. Nat'l Acad. Sci. USA 95:6037 (1998); and Osbourn et al., Immunotechnology 2:181 (1996)).

Antibodies can be humanized using a variety of techniques known in the art including, for example, CDR-grafting (EP 239,400; WO91/09967; U.S. Pat. Nos. 5,225,539; 5,530,101; and 5,585,089), veneering or resurfacing (EP 592,106; EP 519,596; Padlan, Molecular Immunol. 28:489 (1991); Studnicka et al., Protein Engineering 7:805 (1994); Roguska. et al., Proc. Nat'l. Acad. Sci. USA 91:969 (1994)), and chain shuffling (U.S. Pat. No. 5,565,332). Human consensus sequences (Padlan, Mol. Immunol. 31:169 (1994); and Padlan, Mol. Immunol. 28:489 (1991)) have previously used to produce humanized antibodies (Carter et al., Proc. Natl. Acad. Sci. USA 89:4285 (1992); and Presta et al, J. Immunol. 151:2623 (1993)).

Methods for producing chimeric antibodies are known in the art (e.g., Morrison, Science 229:1202 (1985); Oi et al., BioTechniques 4:214 (1986); Gillies et al., J. Immunol. Methods 125:191 (1989); and U.S. Pat. Nos. 5,807,715; 4,816,567; and 4,816,397). Chimeric antibodies in which a variable domain from an antibody of one species is substituted for the variable domain of another species are described, for example, in Munro, Nature 312:597 (1984); Neuberger et al., Nature 312:604 (1984); Sharon et al., Nature 309:364 (1984); Morrison et al., Proc. Nat'l. Acad. Sci. USA 81:6851 (1984); Boulianne et al., Nature 312:643 (1984); Capon et al., Nature 337:525 (1989); and Traunecker et al., Nature 339:68 (1989).

Suitable techniques that additionally may be employed in antibody methods include affinity purification, non-denaturing gel purification, HPLC or RP-HPLC, size exclusion, purification on protein A column, or any combination of these techniques. The antibody isotype can be determined using an ELISA assay, for example, a human Ig can be identified using mouse Ig-absorbed anti-human Ig.

In accordance with the invention, further provided are methods of producing antibodies and functional fragments. In one embodiment, a method includes administering a PM-2 antigen, or cell expressing a PM-2 antigen, to an animal, screening the animal for expression of an antibody that binds to the PM-2 antigen or cell expressing a PM-2 antigen, selecting an animal that produces an antibody that binds to PM-2 antigen or cell expressing a PM-2 antigen, and isolating the antibody from the selected animal. In another embodiment, a method includes administering PM-2 antigen or cell expressing a PM-2 antigen to an animal capable of expressing a human immunoglobulin; isolating spleen cells from an animal that produces antibody that binds to the PM-2 antigen or cell expressing a PM-2 antigen, fusing the spleen cells with a myeloma cell to produce a hybridoma, and screening the hybridoma for expression of an antibody that binds to PM-2 antigen or cell expressing a PM-2 antigen.

In accordance with the invention, there are provided host cells that express antibodies and functional fragments of the antibodies as set forth herein. In particular embodiments, host cells are purified or isolated, and optionally have not been transformed with a nucleic acid that encodes the expressed antibody or functional fragment. In additional embodiments, a host cell expresses an antibody or functional fragment that includes a heavy or light chain sequence with 50%, 60%, 70%, 75%, 80%, 85%, 90%, 95%, 96%, 97%, 98%, or more sequence identity to PM-2 antibody, produced by a cell line DSMZ Deposit No. DSM ACC 2600, or represented by heavy and light chain sequences set forth as SEQ ID NOs:1 and 2. In further embodiments, a host cell expresses a heavy or light chain sequence with at least 80-85%, 85-90%, 90-95%, 95-100% identity to one or more CDRs in heavy chain variable region sequence or light chain variable region sequence of PM-2 antibody, produced by a cell line DSMZ Deposit No. DSM ACC 2600, or represented by heavy and light chain sequences set forth as SEQ ID NOs:1 and 2 (e.g., amino acids 11-18, 36-43, or 82-100 of SEQ ID NO:1, and amino acids 26-34, 52-58, or 97-103 of SEQ ID NO:2).

In accordance with the invention, there are provided isolated and purified nucleic acids. Nucleic acids of the invention include, among other things, nucleic acid sequences 1) encoding antibodies and functional fragments that are structurally or functionally related to PM-2 antibody, produced by a cell line DSMZ Deposit No. DSM ACC 2600, or represented by heavy and light chain sequences set forth as SEQ ID NOs:1 and 2; 2) encode PM-2 antibody, produced by a cell line DSMZ Deposit No. DSM ACC 2600, or represented by heavy and light chain sequences set forth as SEQ ID NOs:1 and 2, or antibodies and functional fragments that include all or a portion of a sequence of SEQ ID NOs:1 or 2 (e.g., one or more CDRs, amino acids 11-18, 36-43, or 82-100 of SEQ ID NO:1, or amino acids 26-34, 52-58, or 97-103 of SEQ ID NO:2, such as nucleotides 31-54, 106-129, and 244-300 of SEQ ID NO:3 and nucleotides 76-102, 154-174, or 289-309 of SEQ ID NO:4); 3) that exhibit a degree of complementarity or identity with nucleic acid sequences encoding antibodies and functional fragments with sequence identity to PM-2 antibody, produced by a cell line DSMZ Deposit No. DSM ACC 2600, or represented by heavy and light chain sequences set forth as SEQ ID NOs:1 and 2; and 4) that hybridize to sequences encoding antibodies and functional fragments that have sequence identity to PM-2 antibody, produced by a cell line DSMZ Deposit No. DSM ACC 2600, or represented by heavy and light chain sequences set forth as SEQ ID NOs:1 and 2, such as nucleotides 31-54, 106-129, and 244-300 of SEQ ID NO:3 and nucleotides 76-102, 154-174, or 289-309 of SEQ ID NO:4.

In particular embodiments, a nucleic acid sequence encodes a heavy or light chain sequence of PM-2 antibody, produced by a cell line DSMZ Deposit No. DSM ACC 2600, or represented by heavy and light chain sequences set forth as SEQ ID NOs:1 and 2, or a functional fragment thereof. In another embodiment, a nucleic acid sequence is 75-100% complementary or identical to a nucleic acid sequence that encodes SEQ ID NO:1. In a further embodiment, a nucleic acid sequence is 75-100% complementary or identical to a nucleic acid sequence that encodes SEQ ID NO:2.

Proteins, such as antibodies that include amino acid substitutions, additions or deletions can be encoded by a nucleic acid. Consequently, nucleic acid sequences encoding proteins that include amino acid substitutions, additions or deletions are also provided.

The terms “nucleic acid” and “polynucleotide” and the like refer to at least two or more ribo- or deoxy-ribonucleic acid base pairs (nucleotides) that are linked through a phosphoester bond or equivalent. Nucleic acids include polynucleotides and polynucleosides. Nucleic acids include single, double or triplex, circular or linear, molecules. Exemplary nucleic acids include but are not limited to: RNA, DNA, cDNA, genomic nucleic acid, naturally occurring and non naturally occurring nucleic acid, e.g., synthetic nucleic acid.

Nucleic acids can be of various lengths. Nucleic acid lengths typically range from about 20 nucleotides to 20 Kb, or any numerical value or range within or encompassing such lengths, 10 nucleotides to 10 Kb, 1 to 5 Kb or less, 1000 to about 500 nucleotides or less in length. Nucleic acids can also be shorter, for example, 100 to about 500 nucleotides, or from about 12 to 25, 25 to 50, 50 to 100, 100 to 250, or about 250 to 500 nucleotides in length, or any numerical value or range or value within or encompassing such lengths. In particular embodiments, a nucleic acid sequence has a length from about 10-20, 20-30, 30-50, 50-100, 100-150, 150-200, 200-250, 250-300, 300-400, 400-500, 500-1000, 1000-2000, nucleotides, or any numerical value or range within or encompassing such lengths. In additional embodiments, nucleic acid sequences range in length to encode SEQ ID NOs:1 or 2, or a subsequence thereof, such as nucleotides 31-54, 106-129, and 244-300 of SEQ ID NO:3 and nucleotides 76-102, 154-174, or 289-309 of SEQ ID NO:4. Shorter polynucleotides are commonly referred to as “oligonucleotides” or “probes” of single- or double-stranded DNA. However, there is no upper limit to the length of such oligonucleotides.

Polynucleotides include L- or D-forms and mixtures thereof, which additionally may be modified to be resistant to degradation when administered to a subject. Particular examples include 5′ and 3′ linkages resistant to endonucleases and exonucleases present in various tissues or fluids of a subject.

In accordance with the invention there are provided nucleic acid sequences that hybridize to a nucleic acid that encodes all or a fragment of a PM-2 antibody, produced by a cell line DSMZ Deposit No. DSM ACC 2600, or represented by heavy and light chain sequences set forth as SEQ ID NOs:1 and 2. In one embodiment, a nucleic acid sequence specifically hybridizes to a nucleic acid encoding SEQ ID NO:1 or a portion thereof (e.g., nucleotides 31-54, 106-129, and 244-300 of SEQ ID NO:3). In another embodiment, a nucleic acid sequence specifically hybridizes to a nucleic acid encoding SEQ ID NO:2 or a portion thereof (e.g., nucleotides 76-102, 154-174, or 289-309 of SEQ ID NO:4). In a further embodiment, a nucleic acid sequence is at least 75-100% complementary or homologous to a nucleic acid sequence that encodes all or a subsequence or fragment of PM-2 antibody, produced by a cell line DSMZ Deposit No. DSM ACC 2600, or represented by heavy and light chain sequences set forth as SEQ ID NOs:1 and 2.

The term “hybridize” and grammatical variations thereof refer to the binding between nucleic acid sequences. Hybridizing sequences will generally have more than about 50% homology (e.g., 50%, 60%, 70%, 80%, 90%, or more identity) to a reference nucleic acid or a sequence complementary to a reference sequence. Hybridizing sequences that are 100% or fully complementary to a reference sequence, for example, to a nucleic acid that encodes an amino acid sequence of a reference sequence, exhibit 100% base pairing with no mismatches. The hybridization region between hybridizing sequences typically is at least about 12-15 nucleotides, 15-20 nucleotides, 20-30 nucleotides, 30-50 nucleotides, 50-100 nucleotides, 100 to 200 nucleotides or more, or any numerical value or range within or encompassing such lengths.

In accordance with the invention, there are further provided antisense polynucleotides, small interfering RNA, and ribozyme nucleic acid. In one embodiment, an antisense polynucleotide, small interfering RNA, or ribozyme nucleic acid specifically hybridizes to a nucleic acid sequence encoding PM-2 antibody, produced by a cell line DSMZ Deposit No. DSM ACC 2600, or represented by heavy and light chain sequences set forth as SEQ ID NOs:1 and 2, or SEQ ID NO:1 or 2 or a portion thereof, and optionally reduces expression of PM-2 antibody, produced by a cell line DSMZ Deposit No. DSM ACC 2600, or represented by heavy and light chain sequences set forth as SEQ ID NOs:1 and 2, or SEQ ID NO:1 or 2. In another embodiment, an antisense polynucleotide, small interfering RNA, or ribozyme nucleic acid is at least 60% or more (e.g., 65%, 70%, 75%, 80%, 85%, 90%, 95%, etc.) complementary or homologous to a nucleic acid sequence that encodes PM-2 antibody, produced by a cell line DSMZ Deposit No. DSM ACC 2600, or represented by heavy and light chain sequences set forth as SEQ ID NOs:1 and 2, or SEQ ID NO:1 or 2, or a subsequence of SEQ ID NOs:1 or 2 (e.g., nucleotides 31-54, 106-129, and 244-300 of SEQ ID NO:3 or nucleotides 76-102, 154-174, or 289-309 of SEQ ID NO:4). Antisense polynucleotides can have a length from about 10-20, 20-30, 30-50, 50-100, 100-150, 150-200, 200-250, 250-300, 300-400, 400-500, 500-1000, 1000-2000 nucleotides, or any numerical value or range within or encompassing such lengths.

As used herein, the term “antisense” refers to a polynucleotide or peptide nucleic acid capable of binding to a specific DNA or RNA sequence. Antisense includes single, double, triple or greater stranded RNA and DNA polynucleotides and peptide nucleic acids (PNAs) that bind RNA transcript or DNA. Particular examples include RNA and DNA antisense that binds to sense RNA. For example, a single stranded nucleic acid can target a protein transcript that participates in metabolism, catabolism, removal or degradation of glycogen from a cell (e.g., mRNA). Antisense molecules are typically 95-100% complementary to the sense strand but can be “partially” complementary, in which only some of the nucleotides bind to the sense molecule (less than 100% complementary, e.g., 95%, 90%, 80%, 70% and sometimes less), or any numerical value or range within or encompassing such percent values.

Triplex forming antisense can bind to double strand DNA thereby inhibiting transcription of the gene. Oligonucleotides derived from the transcription initiation site of the gene, e.g., between positions −10 and +10 from the start site, are one particular example.

Short interfering RNA (referred to as siRNA or RNAi) for inhibiting gene expression is known in the art (see, e.g., Kennerdell et al., Cell 95:1017 (1998); Fire et al., Nature, 391:806 (1998); WO 02/44321; WO 01/68836; WO 00/44895, WO 99/32619, WO 01/75164, WO 01/92513, WO 01/29058, WO 01/89304, WO 02/16620; and WO 02/29858). RNAi silencing can be induced by a nucleic acid encoding an RNA that forms a “hairpin” structure or by expressing RNA from each end of an encoding nucleic acid, making two RNA molecules that hybridize.

Ribozymes, which are enzymatic RNA molecules that catalyze the specific cleavage of RNA can be used to inhibit expression of the encoded protein. Ribozymes form sequence-specific hybrids with complementary target RNA, which is then cleaved. Specific examples include engineered hammerhead motif ribozyme molecules that can specifically and efficiently catalyze endonucleolytic cleavage of sequences encoding a protein that participates in metabolism, catabolism, removal or degradation of glycogen, for example.

Antisense, ribozymes, RNAi and triplex forming nucleic acid are referred to collectively herein as “inhibitory nucleic acid” or “inhibitory polynucleotides.” Such inhibitory nucleic acid or polynucleotides can inhibit or reduce expression of the sequence to which it binds or targets, and consequently, encoded protein as appropriate.

Inhibitory polynucleotides do not require expression control elements in order to function in vivo. Inhibitory polynucleotides can be absorbed by the cell or enter the cell via passive diffusion. Inhibitory polynucleotides can optionally be introduced into a cell using a vector. Inhibitory polynucleotides may be encoded by a nucleic acid so that it is transcribed. Furthermore, a nucleic acid encoding an inhibitory polynucleotide may be operatively linked to an expression control element for sustained or increased expression of the encoded antisense in cells or in vivo. Inhibitory nucleic acid can be designed based upon protein and nucleic acid sequences disclosed herein or available in the database.

Nucleic acid sequences further include nucleotide and nucleoside substitutions, additions and deletions, as well as derivatized forms and fusion/chimeric sequences (e.g., encoding recombinant polypeptide). For example, due to the degeneracy of the genetic code, nucleic acids include sequences and subsequences degenerate with respect to nucleic acids that encode, modified forms and variants thereof. Other examples are nucleic acids complementary to a sequence that encodes Nucleic acid deletions (subsequences and fragments) can have from about 10 to 25, 25 to 50 or 50 to 100 nucleotides. Such nucleic acids are useful for expressing polypeptide subsequences, for genetic manipulation (as primers and templates for PCR amplification), and as probes to detect the presence or an amount of a sequence encoding a protein (e.g., via hybridization), in a cell, culture medium, biological sample (e.g., tissue, organ, blood or serum), or in a subject.

Nucleic acids can be produced using various standard cloning and chemical synthesis techniques. Techniques include, but are not limited to nucleic acid amplification, e.g., polymerase chain reaction (PCR), with genomic DNA or cDNA targets using primers (e.g., a degenerate primer mixture) capable of annealing to antibody encoding sequence. Nucleic acids can also be produced by chemical synthesis (e.g., solid phase phosphoramidite synthesis) or transcription from a gene. The sequences produced can then be translated in vitro, or cloned into a plasmid and propagated and then expressed in a cell (e.g., a host cell such as yeast or bacteria, a eukaryote such as an animal or mammalian cell or in a plant).

In accordance with the invention, there are further provided vectors that comprise nucleic acid sequences of the invention. In one embodiment, a vector includes a nucleic acid sequence encoding an antibody or functional fragment as set forth herein. In another embodiment, a vector includes a nucleic acid sequence encoding

Vectors include viral, prokaryotic (bacterial) and eukaryotic (plant, fungal, mammalian) vectors. Vectors can be used for expression of nucleic acids in vitro or in vivo. Such vectors, referred to as “expression vectors,” are useful for introducing nucleic acids, including nucleic acids that encode PM-2 antibody, produced by a cell line DSMZ Deposit No. DSM ACC 2600, or represented by heavy and light chain sequences set forth as SEQ ID NOs:1 and 2, subsequences and fragments thereof, nucleic acids that encode modified forms or variants of PM-2 antibody, produced by a cell line DSMZ Deposit No. DSM ACC 2600, or represented by heavy and light chain sequences set forth as SEQ ID NOs:1 and 2, nucleic acids that encode inhibitory nucleic acid, and expressing the encoded protein or inhibitory nucleic acid (e.g., in solution or in solid phase), in cells or in a subject in vivo.

Vectors can also be used for manipulation of nucleic acids. For genetic manipulation “cloning vectors” can be employed, and to transcribe or translate the inserted nucleic acid.

A vector generally contains an origin of replication for propagation in a cell in vitro or in vivo. Control elements, including expression control elements, present within a vector, can be included to facilitate transcription and translation, as appropriate.

Vectors can include a selection marker. A “selection marker” is a gene that allows for the selection of cells containing the gene. “Positive selection” refers to a process in which cells that contain the selection marker survive upon exposure to the positive selection. Drug resistance is one example of a positive selection marker-cells containing the marker will survive in culture medium containing the selection drug, and cells lacking the marker will die. Selection markers include drug resistance genes such as neo, which confers resistance to G418; hygr, which confers resistance to hygromycin; and puro, which confers resistance to puromycin. Other positive selection marker genes include genes that allow identification or screening of cells containing the marker. These genes include genes for fluorescent proteins (GFP and GFP-like chromophores, luciferase), the lacZ gene, the alkaline phosphatase gene, and surface markers such as CD8, among others. “Negative selection” refers to a process in which cells containing a negative selection marker are killed upon exposure to an appropriate negative selection agent. For example, cells which contain the herpes simplex virus-thymidine kinase (HSV-tk) gene (Wigler et al., Cell 11:223 (1977)) are sensitive to the drug gancyclovir (GANC). Similarly, the gpt gene renders cells sensitive to 6-thioxanthine.

Viral vectors include those based upon retroviral (lentivirus for infecting dividing as well as non-dividing cells), foamy viruses (U.S. Pat. Nos. 5,624,820, 5,693,508, 5,665,577, 6,013,516 and 5,674,703; WO92/05266 and WO92/14829), adenovirus (U.S. Pat. Nos. 5,700,470, 5,731,172 and 5,928,944), adeno-associated virus (AAV) (U.S. Pat. No. 5,604,090), herpes simplex virus vectors (U.S. Pat. No. 5,501,979), cytomegalovirus (CMV) based vectors (U.S. Pat. No. 5,561,063), reovirus, rotavirus genomes, simian virus 40 (SV40) or papilloma virus (Cone et al., Proc. Natl. Acad. Sci. USA 81:6349 (1984); Eukaryotic Viral Vectors, Cold Spring Harbor Laboratory, Gluzman ed., 1982; Sarver et al., Mol. Cell. Biol. 1:486 (1981); U.S. Pat. No. 5,719,054). Adenovirus efficiently infects slowly replicating and/or terminally differentiated cells and can be used to target slowly replicating and/or terminally differentiated cells. Additional viral vectors useful for expression include parvovirus, Norwalk virus, coronaviruses, paramyxo- and rhabdoviruses, togavirus (e.g., sindbis virus and semliki forest virus) and vesicular stomatitis virus (VSV).

A nucleic acid can be expressed when the nucleic acid is operably linked to an expression control element. As used herein, the term “operably linked” refers to a physical or a functional relationship between the elements referred to that permit them to operate in their intended fashion. Thus, an expression control element “operably linked” to a nucleic acid means that the control element modulates nucleic acid transcription and as appropriate, translation of the transcript.

The term “expression control element” refers to nucleic acid that influences expression of an operably linked nucleic acid. Promoters and enhancers are particular non-limiting examples of expression control elements. A “promoter sequence” is a DNA regulatory region capable of initiating transcription of a downstream (3′ direction) sequence. The promoter sequence includes nucleotides that facilitate transcription initiation. Enhancers also regulate gene expression, but can function at a distance from the transcription start site of the gene to which it is operably linked. Enhancers function at either 5′ or 3′ ends of the gene, as well as within the gene (e.g., in introns or coding sequences). Additional expression control elements include leader sequences and fusion partner sequences, internal ribosome binding sites (IRES) elements for the creation of multigene, or polycistronic, messages, splicing signal for introns, maintenance of the correct reading frame of the gene to permit in-frame translation of mRNA, polyadenylation signal to provide proper polyadenylation of the transcript of interest, and stop codons.

Expression control elements include “constitutive” elements in which transcription of an operably linked nucleic acid occurs without the presence of a signal or stimuli. Expression control elements that confer expression in response to a signal or stimuli, which either increase or decrease expression of operably linked nucleic acid, are “regulatable.” A regulatable element that increases expression of operably linked nucleic acid in response to a signal or stimuli is referred to as an “inducible element.” A regulatable element that decreases expression of the operably linked nucleic acid in response to a signal or stimuli is referred to as a “repressible element” (i.e., the signal decreases expression; when the signal is removed or absent, expression is increased).

Expression control elements include elements active in a particular tissue or cell type, referred to as “tissue-specific expression control elements.” Tissue-specific expression control elements are typically more active in specific cell or tissue types because they are recognized by transcriptional activator proteins, or other transcription regulators active in the specific cell or tissue type, as compared to other cell or tissue types.

Tissue-specific expression control elements include promoters and enhancers active in hyperproliferative cells, such as cell proliferative disorders including neoplasias, tumors and cancers, and metastasis. Particular non-limiting examples of such promoters are hexokinase II, COX-2, alpha-fetoprotein, carcinoembryonic antigen, DE3/MUC1, prostate specific antigen, C-erB2/neu, telomerase reverse transcriptase and hypoxia-responsive promoter.

For bacterial expression, constitutive promoters include T7, as well as inducible promoters such as pL of bacteriophage λ, plac, ptrp, ptac (ptrp-lac hybrid promoter). In insect cell systems, constitutive or inducible promoters (e.g., ecdysone) may be used. In yeast, constitutive promoters include, for example, ADH or LEU2 and inducible promoters such as GAL (see, e.g., Ausubel et al., In: Current Protocols in Molecular Biology, Vol. 2, Ch. 13, ed., Greene Publish. Assoc. & Wiley Interscience, 1988; Grant et al., In: Methods in Enzymology, 153:516-544 (1987), eds. Wu & Grossman, 1987, Acad. Press, N.Y.; Glover, DNA Cloning, Vol. II, Ch. 3, IRL Press, Wash., D.C., 1986; Bitter, In: Methods in Enzymology, 152:673-684 (1987), eds. Berger & Kimmel, Acad. Press, N.Y.; and, Strathern et al., The Molecular Biology of the Yeast Saccharomyces eds. Cold Spring Harbor Press, Vols. I and II (1982)).

For mammalian expression, constitutive promoters of viral or other origins may be used. For example, SV40, or viral long terminal repeats (LTRs) and the like, or inducible promoters derived from the genome of mammalian cells (e.g., metallothionein IIA promoter; heat shock promoter, steroid/thyroid hormone/retinoic acid response elements) or from mammalian viruses (e.g., the adenovirus late promoter; mouse mammary tumor virus LTR) are used.

In accordance with the invention, there are provided host cells transformed or transfected with nucleic acids and vectors of the invention. In one embodiment, a cell is stably or transiently transformed with a nucleic acid that encodes an antibody, a functional fragment, a heavy or light chain sequence, or a portion of a heavy or light chain sequence (e.g., a variable region, or one or more CDRs, amino acids 11-18, 36-43, or 82-100 of SEQ ID NO:1, or amino acids 26-34, 52-58, or 97-103 of SEQ ID NO:2, such as nucleotides 31-54, 106-129, and 244-300 of SEQ ID NO:3 or nucleotides 76-102, 154-174, or 289-309 of SEQ ID NO:4). In another embodiment, a host cell is stably or transiently transformed with an antisense or inhibitory nucleic acid.

Host cells include but are not limited to prokaryotic and eukaryotic cells such as bacteria, fungi (yeast), plant, insect, and animal (e.g., mammalian, including primate and human) cells. The cells may be a primary cell isolate, cell culture (e.g., passaged, established or immortalized cell line), or part of a plurality of cells, or a tissue or organ ex vivo or in a subject (in vivo). For example, bacteria transformed with recombinant bacteriophage nucleic acid, plasmid nucleic acid or cosmid nucleic acid expression vectors; yeast transformed with recombinant yeast expression vectors; plant cell systems infected with recombinant virus expression vectors (e.g., cauliflower mosaic virus, CaMV; tobacco mosaic virus, TMV) or transformed with recombinant plasmid expression vectors (e.g., Ti plasmid); insect cell systems infected with recombinant virus expression vectors (e.g., baculovirus); and animal cell systems infected with recombinant virus expression vectors (e.g., retroviruses, adenovirus, vaccinia virus), or transformed animal cell systems engineered for stable expression.

The term “transformed” or “transfected” when use in reference to a cell (e.g., a host cell) or organism, means a genetic change in a cell following incorporation of an exogenous molecule, for example, a protein or nucleic acid (e.g., a transgene) into the cell. Thus, a “transfected” or “transformed” cell is a cell into which, or a progeny thereof in which an exogenous molecule has been introduced by the hand of man, for example, by recombinant DNA techniques.

The nucleic acid can be stably or transiently transfected or transformed (expressed) in the cell and progeny thereof. Host cells therefore include those that stably or transiently express antibody, functional fragment or nucleic acid. The cell(s) can be propagated and the introduced antibody expressed, or nucleic acid transcribed. A progeny of a transfected or transformed cell may not be identical to the parent cell, since there may be mutations that occur during replication.

Typically, cell transfection or transformation employs a “vector,” which refers to a plasmid, virus, such as a viral vector, or other vehicle known in the art that can be manipulated by insertion or incorporation of a nucleic acid.

A viral particle or vesicle can be designed to be targeted to particular cell types (e.g., hyperproliferating cells) by inclusion of a protein on the surface that binds to a target cell ligand or receptor. Alternatively, a cell type-specific promoter and/or enhancer can be included in the vector in order to express the nucleic acid in target cells. Thus, the viral particle or vesicle itself, viral vector, or a protein on the viral surface can be made to target cells for transfection or transformation in vitro, ex vivo or in vivo.

Introduction of compositions (e.g., protein and nucleic acid) into target cells (e.g., host cells) can also be carried out by methods known in the art such as osmotic shock (e.g., calcium phosphate), electroporation, microinjection, cell fusion, etc. Introduction of nucleic acid and polypeptide in vitro, ex vivo and in vivo can also be accomplished using other techniques. For example, a polymeric substance, such as polyesters, polyamine acids, hydrogel, polyvinyl pyrrolidone, ethylene-vinylacetate, methylcellulose, carboxymethylcellulose, protamine sulfate, or lactide/glycolide copolymers, polylactide/glycolide copolymers, or ethylenevinylacetate copolymers. A nucleic acid can be entrapped in microcapsules prepared by coacervation techniques or by interfacial polymerization, for example, by the use of hydroxymethylcellulose or gelatin-microcapsules, or poly (methylmethacrylate) microcapsules, respectively, or in a colloid system. Colloidal dispersion systems include macromolecule complexes, nano-capsules, microspheres, beads, and lipid-based systems, including oil-in-water emulsions, micelles, mixed micelles, and liposomes.

Liposomes for introducing various compositions into cells are known in the art and include, for example, phosphatidylcholine, phosphatidylserine, lipofectin and DOTAP (e.g., U.S. Pat. Nos. 4,844,904, 5,000,959, 4,863,740, and 4,975,282; and GIBCO-BRL, Gaithersburg, Md.). Piperazine based amphilic cationic lipids useful for gene therapy also are known (see, e.g., U.S. Pat. No. 5,861,397). Cationic lipid systems also are known (see, e.g., U.S. Pat. No. 5,459,127). Polymeric substances, microcapsules and colloidal dispersion systems such as liposomes are collectively referred to herein as “vesicles.” Accordingly, viral and non-viral vector means of delivery into cells, tissue or organs, in vitro, in vivo and ex vivo are included.

The invention includes in vivo methods. For example, a cell such as an undesirably proliferating cell or cell proliferative disorder to which PM-2 antibody or functional fragment binds can be present in a subject, such as a mammal (e.g., a human subject). A subject having such cells may therefore be treated by administering, for example, an antibody, or subsequence or fragment thereof, that binds to such cells.

In accordance with the invention, there are provided methods of treating undesirable cell proliferation or a cell proliferative or cellular hyperproliferative disorder in a subject. Such methods can be practiced with any of the antibodies, functional fragments, modified and variant forms set forth herein. In one embodiment, a method includes administering to a subject an amount of PM-2 antibody, produced by a cell line DSMZ Deposit No. DSM ACC 2600, or represented by heavy and light chain sequences set forth as SEQ ID NOs:1 and 2, effective to treat the undesirable cell proliferation or a cell proliferative or cell hyperproliferative disorder in the subject.

As used herein, the terms “cell proliferative disorder” and “cellular hyperproliferative disorder” and grammatical variations thereof, when used in reference to a cell, tissue or organ, refers to any undesirable, excessive or abnormal cell, tissue or organ growth, proliferation, differentiation or survival. A hyperproliferative cell denotes a cell whose growth, proliferation, or survival is greater than desired, such as a reference normal cell, e.g., a cell that is of the same tissue or organ but is not a hyperproliferative cell, or a cell that fails to differentiate normally. Undesirable cell proliferation and hyperproliferative disorders include diseases and physiological conditions, both benign hyperplastic conditions characterized by undesirable, excessive or abnormal cell numbers, cell growth, cell proliferation, cell survival or differentiation in a subject. Specific examples of such disorders include metastatic and non-metastatic neoplasia, tumors and cancers (malignancies).

In various embodiments, a method includes administering to a subject a PM-2 antibody, produced by a cell line DSMZ Deposit No. DSM ACC 2600, or represented by heavy and light chain sequences set forth as SEQ ID NOs:1 and 2, in an amount effective to treat the cell proliferative or cellular hyperproliferative disorder in the subject. In particular aspects, the disorder is a neoplasia, tumor or metastatic or non-metastatic cancer (malignancy). In additional aspects, the disorder affects or is present in part at least in breast, lung, thyroid, head and neck, nasopharynx, nose or sinuses, brain, spine, adrenal gland, thyroid, lymph, gastrointestinal (mouth, esophagus, stomach, duodenum, ileum, jejunum (small intestine), colon, rectum), genito-urinary tract (uterus, ovary, endometrium, cervix, bladder, testicle, penis, prostate), kidney, pancreas, adrenal gland, liver, bone, bone marrow, lymph, blood, muscle, skin, or the hematopoetic system.

The terms “tumor,” “cancer” and “neoplasia” are used interchangeably and refer to a cell or population of cells whose growth, proliferation or survival is greater than growth, proliferation or survival of a normal counterpart cell, e.g. a cell proliferative or differentiative disorder. Typically, the growth is uncontrolled. The term “malignancy” refers to invasion of nearby tissue. The term “metastasis” refers to spread or dissemination of a tumor, cancer or neoplasia to other sites, locations or regions within the subject, in which the sites, locations or regions are distinct from the primary tumor or cancer.

Invention methods can be used to reduce or inhibit metastasis of a primary tumor or cancer to other sites, or the formation or establishment of metastatic tumors or cancers at other sites distal from the primary tumor or cancer thereby inhibiting or reducing tumor or cancer relapse or tumor or cancer progression. Thus, methods of the invention include, among other things, 1) reducing or inhibiting growth, proliferation, mobility or invasiveness of tumor or cancer cells that potentially or do develop metastases (e.g., disseminated tumor cells, DTC); 2) reducing or inhibiting formation or establishment of metastases arising from a primary tumor or cancer to one or more other sites, locations or regions distinct from the primary tumor or cancer; 3) reducing or inhibiting growth or proliferation of a metastasis at one or more other sites, locations or regions distinct from the primary tumor or cancer after a metastasis has formed or has been established; and 4) reducing or inhibiting formation or establishment of additional metastasis after the metastasis has been formed or established.

Neoplasias, tumors and cancers include a sarcoma, carcinoma, adenocarcinoma, melanoma, myeloma, blastoma, glioma, lymphoma or leukemia. Exemplary cancers include, for example, carcinoma, sarcoma, adenocarcinoma, melanoma, neural (blastoma, glioma), mesothelioma and reticuloendothelial, lymphatic or haematopoietic neoplastic disorders (e.g., myeloma, lymphoma or leukemia). In particular aspects, a neoplasia, tumor or cancer includes a lung adenocarcinoma, lung carcinoma, diffuse or interstitial gastric carcinoma, colon adenocarcinoma, prostate adenocarcinoma, esophagus carcinoma, breast carcinoma, pancreas adenocarcinoma, ovarian adenocarcinoma, adenocarcinoma of the adrenal gland, adenocarcinoma of the endometrium or uterine adenocarcinoma.

Neoplasia, tumors and cancers include benign, malignant, metastatic and non-metastatic types, and include any stage (I, II, III, IV or V) or grade (G1, G2, G3, etc.) of neoplasia, tumor, or cancer, or a neoplasia, tumor, cancer or metastasis that is progressing, worsening, stabilized or in remission.

Neoplasias, tumors and cancers can arise from a multitude of primary tumor types, including but not limited to breast, lung, thyroid, head and neck, nasopharynx, nose or sinuses, brain, spine, adrenal gland, thyroid, lymph, gastrointestinal (mouth, esophagus, stomach, duodenum, ileum, jejunum (small intestine), colon, rectum), genito-urinary tract (uterus, ovary, endometrium, cervix, bladder, testicle, penis, prostate), kidney, pancreas, adrenal gland, liver, bone, bone marrow, lymph, blood, muscle, skin, and the hematopoetic system, and may metastasize to secondary sites.

A “solid neoplasia, tumor or cancer” refers to neoplasia, tumor or cancer (e.g., metastasis) that typically aggregates together and forms a mass. Specific examples include visceral tumors such as melanomas, breast, pancreatic, uterine and ovarian cancers, testicular cancer, including seminomas, gastric or colon cancer, hepatomas, adrenal, renal and bladder carcinomas, lung, head and neck cancers and brain tumors/cancers.

Carcinomas refer to malignancies of epithelial or endocrine tissue, and include respiratory system carcinomas (lung, small cell lung), gastrointestinal system carcinomas, genitourinary system carcinomas, testicular carcinomas, breast carcinomas, prostatic carcinomas, endocrine system carcinomas, and melanomas. The term also includes carcinosarcomas, e.g., which include malignant tumors composed of carcinomatous and sarcomatous tissues. Adenocarcinoma includes a carcinoma of a glandular tissue, or in which the tumor forms a gland like structure. Melanoma refers to malignant tumors of melanocytes and other cells derived from pigment cell origin that may arise in the skin, the eye (including retina), or other regions of the body. Additional carcinomas can form from the uterine/cervix, endometrium, lung, head/neck, colon, pancreas, testes, adrenal gland, kidney, esophagus, stomach, liver and ovary.

Sarcomas refer to malignant tumors of mesenchymal cell origin. Exemplary sarcomas include for example, lymphosarcoma, liposarcoma, osteosarcoma, chondrosarcoma, leiomyosarcoma, rhabdomyosarcoma and fibrosarcoma.

Neural neoplasias include glioma, glioblastoma, meningioma, neuroblastoma, retinoblastoma, astrocytoma, oligodendrocytoma

Specific non-limiting examples of neoplasias, tumors and cancers amenable to treatment include malignant and non-malignant neoplasias, tumors and cancers, and metastasis. In particular, a neoplasia, tumor, cancer or metastasis of any stage (e.g., stages IA, IB, IIA, IIB, IIIA, IIIB or IV) or grade (e.g., grades G1, G2 or G3). Additional non-limiting examples include a stomach adenocarcinoma, colorectal adenocarcinoma, squamous cell lung carcinoma, lung adenocarcinoma, squamous cell carcinoma of the esophagus, adenocarcinoma of the pancreas, urothel carcinoma of the urinary bladder, renal carcinoma of the kidney, adenocarcinoma of the prostate, ductal carcinoma of the breast, lobular carcinoma of the breast, adenocarcinoma of the ovary, adenocarcinoma of the adrenal gland, adenocarcinoma of the endometrium or a uterus adenocarcinoma.

A “liquid neoplasia, tumor or cancer” refers to a neoplasia, tumor or cancer of the reticuloendothelial or hematopoetic system, such as a lymphoma, myeloma, or leukemia, or a neoplasia that is diffuse in nature. Particular examples of leukemias include acute and chronic lymphoblastic, myeloblastic and multiple myeloma. Typically, such diseases arise from poorly differentiated acute leukemias, e.g., erythroblastic leukemia and acute megakaryoblastic leukemia. Specific myeloid disorders include, but are not limited to, acute promyeloid leukemia (APML), acute myelogenous leukemia (AML) and chronic myelogenous leukemia (CML); lymphoid malignancies include, but are not limited to, acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Specific malignant lymphomas include, non-Hodgkin lymphoma and variants, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

As used herein, the terms “treat,” “treating,” “treatment” and grammatical variations thereof mean subjecting an individual patient to a protocol, regimen, process or remedy, in which it is desired to obtain a physiologic response or outcome in that patient. Since every treated patient may not respond to a particular treatment protocol, regimen, process or remedy, treating does not require that the desired physiologic response or outcome be achieved in each and every patient or patient population. Accordingly, a given patient or patient population may fail to respond or respond inadequately to treatment.

Methods of the invention may be practiced by any mode of administration or by any route, systemic, regional and local administration. Exemplary administration routes include intravenous, intraarterial, intradermal, intramuscular, subcutaneous, intra-pleural, transdermal (topical), transmucosal, intra-cranial, intra-spinal, intra-ocular, rectal, oral (alimentary) and mucosal.

Methods of the invention include, among other things, methods that provide a detectable or measurable improvement in a condition of a given subject, such as alleviating or ameliorating one or more adverse (physical) symptoms or consequences associated with the presence of a cell proliferative or cellular hyperproliferative disorder, neoplasia, tumor or cancer, or metastasis, i.e., a therapeutic benefit or a beneficial effect.

A therapeutic benefit or beneficial effect is any objective or subjective, transient, temporary, or long-term improvement in the condition or pathology, or a reduction in onset, severity, duration or frequency of an adverse symptom associated with or caused by cell proliferation or a cellular hyperproliferative disorder such as a neoplasia, tumor or cancer, or metastasis. A satisfactory clinical endpoint of a treatment method in accordance with the invention is achieved, for example, when there is an incremental or a partial reduction in severity, duration or frequency of one or more associated pathologies, adverse symptoms or complications, or inhibition or reversal of one or more of the physiological, biochemical or cellular manifestations or characteristics of cell proliferation or a cellular hyperproliferative disorder such as a neoplasia, tumor or cancer, or metastasis. A therapeutic benefit or improvement therefore be a cure, such as destruction of target proliferating cells (e.g., neoplasia, tumor or cancer, or metastasis) or ablation of one or more, most or all pathologies, adverse symptoms or complications associated with or caused by cell proliferation or the cellular hyperproliferative disorder such as a neoplasia, tumor or cancer, or metastasis. However, a therapeutic benefit or improvement need not be a cure or complete destruction of all target proliferating cells (e.g., neoplasia, tumor or cancer, or metastasis) or ablation of all pathologies, adverse symptoms or complications associated with or caused by cell proliferation or the cellular hyperproliferative disorder such as a neoplasia, tumor or cancer, or metastasis. For example, partial destruction of a tumor or cancer cell mass, or a stabilization of the tumor or cancer mass, size or cell numbers by inhibiting progression or worsening of the tumor or cancer, can reduce mortality and prolong lifespan even if only for a few days, weeks or months, even though a portion or the bulk of the tumor or cancer mass, size or cells remain.

Specific non-limiting examples of therapeutic benefit include a reduction in neoplasia, tumor or cancer, or metastasis volume (size or cell mass) or numbers of cells, inhibiting or preventing an increase in neoplasia, tumor or cancer volume (e.g., stabilizing), slowing or inhibiting neoplasia, tumor or cancer progression, worsening or metastasis, stimulating, inducing or increasing neoplasia, tumor or cancer cell lysis or apoptosis or inhibiting neoplasia, tumor or cancer proliferation, growth or metastasis. An invention method may not take effect immediately. For example, treatment may be followed by an increase in the neoplasia, tumor or cancer cell numbers or mass, but over time eventual stabilization or reduction in tumor cell mass, size or numbers of cells in a given subject may subsequently occur after cell lysis or apoptosis of the neoplasia, tumor or cancer, or metastasis.

Additional adverse symptoms and complications associated with neoplasia, tumor, cancer and metastasis that can be inhibited, reduced, decreased, delayed or prevented include, for example, nausea, lack of appetite, lethargy, pain and discomfort. Thus, a partial or complete decrease or reduction in the severity, duration or frequency of an adverse symptom or complication associated with or caused by a cellular hyperproliferative disorder, an improvement in the subjects well being, such as increased energy, appetite, psychological well being, are all particular non-limiting examples of therapeutic benefit. A therapeutic benefit or improvement therefore can also include a subjective improvement in the quality of life of a treated subject.

In various embodiments, a method reduces or decreases neoplasia, tumor or cancer, or metastasis size or volume, inhibits or prevents an increase in neoplasia, tumor or cancer, metastasis size or volume, inhibits or delays neoplasia, tumor or cancer progression or worsening, stimulates neoplasia, tumor or cancer, or metastasis cell lysis or apoptosis, or inhibits, reduces, decreases or delays neoplasia, tumor or cancer proliferation or metastasis. In an additional embodiment, a method prolongs or extends lifespan of the subject. In a further embodiment, a method improves the quality of life of the subject.

Examination of a biopsied sample containing a neoplasia, tumor or cancer, or metastasis (e.g., blood or tissue sample), can establish neoplastic, tumor or cancer, or metastasis cell volume or cell numbers, and therefore whether a reduction or stabilization in mass or numbers or volume of neoplastic, tumor or cancer or metastatic cells or inhibition of neoplasia, tumor, cancer or metastatic cell establishment, formation, proliferation, growth or survival (apoptosis) has occurred. For a solid neoplasia, tumor or cancer, invasive and non-invasive imaging methods can ascertain neoplasia, tumor or cancer size or volume. Examination of blood or serum, or bone marrow, for example, for populations, numbers and types of cells (e.g., hematopoetic cellular hyperproliferative disorders, disseminated tumor cells) can establish whether a reduction or stabilization in mass or numbers of neoplastic, tumor, cancer or metastasis cells or inhibition of neoplastic, tumor, cancer or metastasis establishment, formation, proliferation, growth or survival (apoptosis) has occurred.

Invention compositions and methods can be combined with any other treatment or therapy that provides a desired effect. In particular, treatments and therapies that have been characterized as having an anti-cell proliferative activity or function are applicable. Exemplary treatments and therapies include anti-cell proliferative or immune enhancing agents or drugs.

The treatments and therapies can be performed prior to, substantially contemporaneously with any other methods of the invention, for example, an anti-cell proliferative or anti-cellular hyperproliferative disorder (e.g., a neoplasia, tumor or cancer, or metastasis).

The invention therefore provides combination methods in which the methods of the invention, in which any of the antibodies, functional fragments, and modified and variant forms, are used in a combination with any therapeutic regimen, treatment protocol or composition, such as an anti-cell proliferative protocol, agent or drug set forth herein or known in the art. In one embodiment, a method includes administering PM-2 antibody, produced by a cell line DSMZ Deposit No. DSM ACC 2600, or represented by heavy and light chain sequences set forth as SEQ ID NOs:1 and 2, and an anti-cell proliferative or immune enhancing treatment, agent or drug. The anti-cell proliferative or immune enhancing treatment, agent or drug can be administered prior to, substantially contemporaneously with or following administration of PM-2 antibody, produced by a cell line DSMZ Deposit No. DSM ACC 2600, or represented by heavy and light chain sequences set forth as SEQ ID NOs:1 and 2.

As used herein, an “anti-cell proliferative,” “anti-neoplastic,” “anti-tumor,” or “anti-cancer” treatment, therapy, activity or effect means any therapy, treatment regimen, agent, drug, protocol or process that is useful in treating pathologies, adverse symptoms or complications associated with or caused by abnormal or undesirable cell proliferation (cell hyperproliferation), a cellular hyperproliferative disorder, neoplasia, tumor or cancer, or metastasis. Particular therapies, treatment regimens, agents, drugs, protocol or processes can inhibit, decrease, slow, reduce, delay, or prevent cell proliferation, cell growth, cellular hyperproliferation, neoplastic, tumor, or cancer (malignant) growth, proliferation, survival or metastasis. Such treatments, therapies, regimens, protocols, agents and drugs, can operate by disrupting, reducing, inhibiting or delaying cell cycle progression or cell proliferation or growth; increasing, stimulating or enhancing cell apoptosis, lysis or death; inhibiting nucleic acid or protein synthesis or metabolism; reducing, decreasing, inhibiting or delaying cell division; or decreasing, reducing or inhibiting cell survival, or production or utilization of a cell survival factor, growth factor or signaling pathway (extracellular or intracellular).

Examples of anti-cell proliferative treatments and therapies include chemotherapy, immunotherapy, radiotherapy (ionizing or chemical), local or regional thermal (hyperthermia) therapy and surgical resection.

Specific non-limiting classes of anti-cell proliferative agents and drugs include alkylating agents, anti-metabolites, plant extracts, plant alkaloids, nitrosoureas, hormones (steroids), nucleoside and nucleotide analogues. Specific non-limiting examples of microbial toxins include bacterial cholera toxin, pertussis toxin, anthrax toxin, diphtheria toxin, and plant toxin ricin. Specific examples of drugs include cyclophosphamide, azathioprine, cyclosporin A, melphalan, chlorambucil, mechlorethamine, busulphan, methotrexate, 6-mercaptopurine, thioguanine, 5-fluorouracil, 5-fluorouridine, cytosine arabinoside, AZT, 5-azacytidine (5-AZC) and 5-azacytidine related compounds, bleomycin, actinomycin D, mithramycin, mitomycin C, carmustine, calicheamicin, lomustine, semustine, streptozotocin, teniposide, etoposide, hydroxyurea, cisplatin, carboplatin, levamisole, mitotane, procarbazine, dacarbazine, taxol, vinblastine, vincristine, vindesine, doxorubicin, daunomycin and dibromomannitol. Specific non-limiting examples of hormones include prednisone, prednisolone, diethylstilbesterol, flutamide, leuprolide, and gonatrophin releasing hormone antagonists.

Radiotherapy includes internal or external delivery to a subject. For example, alpha, beta, gamma and X-rays can administered to the subject externally without the subject internalizing or otherwise physically contacting the radioisotope. Specific examples of X-ray dosages range from daily doses of 50 to 200 roentgens for prolonged periods of time (3 to 5/week), to single doses of 2000 to 6000 roentgens. Dosages vary widely, and depend on duration of exposure, the half-life of the isotope, the type of radiation emitted, the cell type and location treated and the progressive stage of the disease. Specific non-limiting examples of radionuclides include, for example, ⁴⁷Sc ⁶⁷Cu, ⁷²Se, ⁸⁸Y, ⁹⁰Sr, ⁹⁰Y, ⁹⁷Ru, ⁹⁹Tc, ¹⁰⁵Rh, ¹¹¹In, ¹²⁵I, ¹³¹I, ¹⁴⁹Tb, ¹⁵³Sm, ¹⁸⁶Re, ¹⁸⁸Re, ¹⁹⁴Os, ²⁰³Pb, ²¹¹At, ²¹²Bi, ²¹³Bi, ²¹²Pb, ²²³Ra, ²²⁵Ac, ²²⁷Ac, and ²²⁸Th.

Antibodies that bind to tumor cells are a particular example of an anti-cell proliferative treatment or therapy. Anti-tumor antibodies include, for example, M195 antibody which binds to leukemia cell CD33 antigen (U.S. Pat. No. 6,599,505); monoclonal antibody DS6 which binds to ovarian carcinoma CA6 tumor-associated antigen (U.S. Pat. No. 6,596,503); human IBD12 monoclonal antibody which binds to epithelial cell surface H antigen (U.S. Pat. No. 4,814,275); and BR96 antibody which binds to Le^{x} carbohydrate epitope expressed by colon, breast, ovary, and lung carcinomas. Additional anti-tumor antibodies that can be employed include, for example, Herceptin (anti-Her-2 neu antibody), Rituxan®, Zevalin, Bevacizumab (Avastin), Bexxar, Campath®, Oncolym, 17-1A (Edrecolomab), 3F8 (anti-neuroblastoma antibody), MDX-CTLA4, IMC-C225 (Cetuximab) and Mylotarg.

As used here, the term “immune enhancing,” when used in reference to a treatment, therapy, agent or drug means that the treatment, therapy, agent or drug provides an increase, stimulation, induction or promotion of an immune response, humoral or cell-mediated. Such therapies can enhance immune response generally, or enhance immune response to a specific target, e.g., a cell proliferative or cellular hyperproliferative disorder such as a neoplasia, tumor or cancer, or metastasis.

Specific non-limiting examples of immune enhancing agents include antibody, cell growth factors, cell survival factors, cell differentiative factors, cytokines, interferons and chemokines. Additional examples of immune enhancing agents and treatments include immune cells such as lymphocytes, plasma cells, macrophages, dendritic cells, NK cells and B-cells that either express antibody against the cell proliferative disorder or otherwise are likely to mount an immune response against the cell proliferative disorder. Cytokines that enhance or stimulate immunogenicity include IL-2, IL-1α, IL-1β, L-3, IL-6, IL-7, granulocyte-macrophage-colony stimulating factor (GMCSF), IFN-γ, IL-12, TNF-α, and TNFβ, which are also non-limiting examples of immune enhancing agents. Chemokines including MIP-1α, MIP-1β, RANTES, SDF-1, MCP-1, MCP-2, MCP-3, MCP-4, eotaxin, eotaxin-2, I-309/TCA3, ATAC, HCC-1, HCC-2, HCC-3, PARC, TARC, LARC/MIP-3α, CKβ, CKβ6, CKβ7, CKβ8, CKβ9, CKβ11, CKβ12, C10, IL-8, ENA-78, GROα, GROβ, GCP-2, PBP/CTAPIIIβ-TG/NAP-2, Mig, PBSF/SDF-1, and lymphotactin are further non-limiting examples of immune enhancing agents.

Methods of the invention also include, among other things, methods that result in a reduced need or use of another treatment protocol or therapeutic regimen, process or remedy. For example, for a neoplasia, tumor or cancer, or metastasis, a method of the invention has a therapeutic benefit if in a given subject it results in a less frequent or reduced dose or elimination of an anti-cell proliferative (e.g., anti-neoplastic, anti-tumor, anti-cancer or anti-metastatic) or immune enhancing treatment or therapy, such as a chemotherapeutic drug, radiotherapy, immunotherapy, or surgery for neoplasia, tumor or cancer, or metastasis treatment or therapy.

In accordance with the invention, methods of reducing need or use of an anti-cell proliferative (e.g., anti-neoplastic, anti-tumor, anti-cancer or anti-metastasis) treatment or therapy are provided. In one embodiment, a method includes administering to a subject PM-2 antibody, produced by a cell line DSMZ Deposit No. DSM ACC 2600, or represented by heavy and light chain sequences set forth as SEQ ID NOs:1 and 2, in an amount effective to treat a cellular hyperproliferative disorder (e.g., a neoplasia, tumor or cancer, or metastasis), and to reduce or eliminate need for an anti-cell proliferative (anti-neoplasia, anti-tumor or anti-cancer, or anti-metastasis) or immune-enhancing therapy. The methods can be performed prior to, substantially contemporaneously with or following administration of an anti-neoplastic, -tumor, -cancer or -metastasis, or immune-enhancing therapy.

The doses or “amount effective” or “amount sufficient” in a method of treatment or therapy in which it is desired to achieve a therapeutic benefit or improvement includes, for example, any objective or subjective alleviation or amelioration of one, several or all pathologies, adverse symptoms or complications associated with or caused by the target (e.g., cellular hyperproliferative disorder), to a measurable or detectable extent, although preventing, inhibiting or delaying a progression or worsening of the target (e.g., cellular hyperproliferative disorder) pathology, adverse symptom or complication, is a satisfactory outcome. Thus, in the case of a cellular hyperproliferative disorder, the amount will be sufficient to provide a therapeutic benefit to a given subject or to alleviate or ameliorate a pathology, adverse symptom or complication of the disorder in a given subject. Single or multiple doses may be administered or the dose may be proportionally increased or reduced as indicated by the status of treatment or therapeutic target (e.g., cellular hyperproliferative disorder) or any side effect(s) of the treatment or therapy.

Exemplary non-limiting amounts (doses) are in a range of about 0.1 mg/kg to about 100 mg/kg, and any numerical value or range or value within such ranges. Greater or lesser amounts (or doses) can be administered, for example, 0.01-500 mg/kg, and any numerical value or range or value within such ranges. Additional exemplary non-limiting amounts (or doses) range from about 0.1-50 mg/kg, 0.5-50 mg/kg, 1.0-25 mg/kg, 1.0-10 mg/kg, and any numerical value or range or value within such ranges.

Methods of the invention may be practiced one or more times (e.g., 1-10, 1-5 or 1-3 times) per day, week, month, or year. The skilled artisan will know when it is appropriate to delay or discontinue administration. An exemplary non-limiting dosage schedule is 1-7 times per week, for 1, 2, 3, 4, 5, 6, 7, 8, 9, 10, 15, 20 or more weeks, and any numerical value or range or value within such ranges.

Of course, as is typical for any treatment or therapy, different subjects will exhibit different responses to treatment and some may not respond or respond inadequately to a particular treatment protocol, regimen or process. Amounts effective or sufficient will therefore depend at least in part upon the disorder treated (e.g., cell proliferation, benign hyperplasia or a neoplasia, tumor or cancer and the type or stage, e.g., the tumor or cancer grade and if it is advanced, late or early stage), the therapeutic effect desired, as well as the individual subject (e.g., the bioavailability within the subject, gender, age, etc.) and the subject's response to the treatment based upon genetic and epigenetic variability (e.g., pharmacogenomics).

Cell toxicity and viability (cell apoptosis, lysis, growth proliferation, etc.) can be measured in a variety of ways on the basis of calorimetric, luminescent, radiometric, or fluorometric assays known in the art. Colorimetric techniques for determining cell viability include, for example, Trypan Blue exclusion. In brief, cells are stained with Trypan Blue and counted using a hemocytometer. Viable cells exclude the dye whereas dead and dying cells take up the blue dye and are easily distinguished under a light microscope. Neutral Red is adsorbed by viable cells and concentrates in cell lysosomes; viable cells can be determined with a light microscope by quantitating numbers of Neutral Red stained cells.

Fluorometric techniques for determining cell viability include, for example, propidium iodide, a fluorescent DNA intercalating agent. Propidium iodide is excluded from viable cells but stains the nucleus of dead cells. Flow cytometry of propidium iodide labeled cells can then be used to quantitate viable and dead cells. Release of lactate dehydrogenase (LDH) indicates structural damage and death of cells, and can be measured by a spectrophotometric enzyme assay. Bromodeoxyuridine (BrdU) is incorporated into newly synthesized DNA and can be detected with a fluorochrome-labeled antibody. The fluorescent dye Hoechst 33258 labels DNA and can be used to quantitate proliferation of cells (e.g., flow cytometry). Quantitative incorporation of the fluorescent dye carboxyfluorescein diacetate succinimidyl ester (CFSE or CFDA-SE) can provide cell division analysis (e.g., flow cytometry). This technique can be used either in vitro or in vivo. 7-aminoactinomycin D (7-AAD) is a fluorescent intercalator that undergoes a spectral shift upon association with DNA, and can provide cell division analysis (e.g., flow cytometry).

Radiometric techniques for determining cell proliferation include, for example, [³H]-Thymidine, which is incorporated into newly synthesized DNA of living cells and frequently used to determine proliferation of cells. Chromium (⁵¹Cr)-release from dead cells can be quantitated by scintillation counting in order to quantitate cell viability.

Luminescent techniques for determining cell viability include, for example, the CellTiter-Glo luminescent cell viability assay (Promega Madison Wis.). This technique quantifies the amount of ATP present to determine the number of viable cells.

Commercially available kits for determining cell viability and cell proliferation include, for example, Cell Proliferation Biotrak ELISA (Amersham Biosciences Piscataway, N.J.); the Guava ViaCount™ Assay, which provides rapid cell counts and viability determination based on differential uptake of fluorescent reagents (Guava Technologies, Hayward, Calif.); the CyQUANT® Cell Proliferation Assay Kit (Molecular Probes, Inc., Eugene, Oreg.); and the CytoLux Assay Kit (PerkinElmer Life Sciences Inc., Boston, Mass.). The DELFIA® Assay Kits (PerkinElmer Life Sciences Inc., Boston, Mass.) can determine cell proliferation and viability using a time-resolved fluorometric method. The Quantos™ Cell Proliferation Assay is a fluorescence-based assay that measures the fluorescence of a DNA-dye complex from lysed cells (Stratagene, La Jolla, Calif.). The CellTiter-Glo cell viability assay is a luminescent assay for measuring cell viability (Promega, Madison Wis.).

The terms “subject” and “patient” are used interchangeably herein and refer to animals, typically mammals, such as humans, non-human primates (gorilla, chimpanzee, orangutan, macaque, gibbon), domestic animals (dog and cat), farm and ranch animals (horse, cow, goat, sheep, pig), laboratory and experimental animals (mouse, rat, rabbit, guinea pig). Subjects include disease model animals (e.g., such as mice, rats and non-human primates) for studying in vivo efficacy (e.g., a neoplasia, tumor or cancer, or metastasis animal model). Human subjects include children, for example, newborns, infants, toddlers and teens, between the ages of 1 and 5, 5 and 10 and 10 and 18 years, adults between the ages of 18 and 60 years, and the elderly, for example, between the ages of 60 and 65, 65 and 70 and 70 and 100 years.

Subjects include mammals (e.g., humans) in need of treatment, that is, they have undesirable or aberrant cell proliferation (cell hyperproliferation) or a cellular hyperproliferative disorder. Subjects also include those at risk of having a undesirable cell proliferation or a cellular hyperproliferative disorder. Subjects further include a subject in need of an anti-cell proliferative or immune enhancing treatment or therapy due to a lab or clinical diagnosis warranting such treatment, subjects undergoing an anti-cell proliferative or immune enhancing therapy, and subjects having undergone an anti-cell proliferative or immune enhancing therapy and are at risk of relapse or recurrence.

At risk subjects include those with a family history, genetic predisposition, or who have suffered a previous affliction with a cell proliferative or cellular hyperproliferative disorder (e.g., a benign hyperplasia, neoplasia, tumor or cancer, or metastasis), and are at risk of relapse or recurrence. At risk subjects further include environmental exposure to carcinogens or mutagens, such as smokers, or those in an occupational (industrial, chemical, agricultural) setting. Such subjects at risk for developing a cell proliferative or cellular hyperproliferative disorder such as neoplasia, tumor or cancer can be identified with genetic screens for tumor associated genes, gene deletions or gene mutations. Subjects that lack Brca1 are at risk for developing breast cancer, for example. Subjects at risk for developing colon cancer have deleted or mutated tumor suppressor genes, such as adenomatous polyposis coli (APC), for example. At risk subjects having particular genetic predisposition towards cell proliferative disorders are known (see, e.g., The Genetic Basis of Human Cancer 2^{nd} ed. by Bert Vogelstein (Editor), Kenneth W. Kinzler (Editor) (2002) McGraw-Hill Professional; The Molecular Basis of Human Cancer. Edited by W B Coleman and G J Tsongalis (2001) Humana Press; and The Molecular Basis of Cancer. Mendelsohn et al., W B Saunders (1995)).

At risk subjects can therefore be treated in order to inhibit or reduce the likelihood of developing a cell proliferative or cellular hyperproliferative disorder, or after having been cured of a cell proliferative disorder, suffering a relapse or recurrence of the same or a different cell proliferative or cellular hyperproliferative disorder. The result of such treatment can be to reduce the risk of developing a cell proliferative or cellular hyperproliferative disorder, or to prevent a cell proliferative or cellular hyperproliferative disorder, or a pathology, adverse symptom or complication thereof in the treated at risk subject.

The invention further provides kits, including antibodies, functional fragments, modified and variants forms, nucleic acids, agents, drugs and pharmaceutical formulations, packaged into suitable packaging material, optionally in combination with instructions for using the kit components, e.g., instructions for performing a method of the invention. In one embodiment, a kit includes a PM-2 antibody, produced by a cell line DSMZ Deposit No. DSM ACC 2600, or represented by heavy and light chain sequences set forth as SEQ ID NOs:1 and 2. In one aspect, the instructions are for treating undesirable cell proliferation or hyperproliferation, or a cellular hyperproliferative disorder. In another aspect, the instructions are for treating a neoplasia, tumor or cancer, or metastasis. In a further embodiment, a kit includes a PM-2 antibody, produced by a cell line DSMZ Deposit No. DSM ACC 2600, or represented by heavy and light chain sequences set forth as SEQ ID NOs:1 and 2, and instructions for treating undesirable cell proliferation or hyperproliferation, or a cellular hyperproliferative disorder, and an anti-cell proliferative or immune enhancing treatment, agent or drug. In various aspects, a kit includes an anti-neoplastic, anti-cancer or anti-tumor agent. In still a further aspects, a kit includes an article of manufacture, for example, an article of manufacture for delivering the antibody or nucleic acid, anti-cell proliferative or immune enhancing treatment, agent or drug into a subject locally, regionally or systemically.

The term “packaging material” refers to a physical structure housing the components of the kit. The packaging material can maintain the components sterilely, and can be made of material commonly used for such purposes (e.g., paper, corrugated fiber, glass, plastic, foil, ampules, etc.). The label or packaging insert can include appropriate written instructions, for example, practicing a method of the invention, e.g., treating a cell proliferative or cellular hyperproliferative disorder, an assay for screening for, detecting or identifying a PM-2 antigen or epitope, or a cell to which PM-2 antibody, produced by a cell line DSMZ Deposit No. DSM ACC 2600, or represented by heavy and light chain sequences set forth as SEQ ID NOs:1 and 2, binds, etc. Thus, in additional embodiments, a kit includes a label or packaging insert including instructions for practicing a method of the invention in solution, in vitro, in vivo, or ex vivo.

Instructions can therefore include instructions for practicing any of the methods of the invention described herein. For example, invention pharmaceutical compositions can be included in a container, pack, or dispenser together with instructions for administration to a subject to treat a cell proliferative or cellular hyperproliferative disorder, such as a neoplasia, tumor or cancer, or metastasis. Instructions may additionally include indications of a satisfactory clinical endpoint or any adverse symptoms or complications that may occur, storage information, expiration date, or any information required by regulatory agencies such as the Food and Drug Administration for use in a human subject.

The instructions may be on “printed matter,” e.g., on paper or cardboard within the kit, on a label affixed to the kit or packaging material, or attached to a vial or tube containing a component of the kit. Instructions may comprise voice or video tape and additionally be included on a computer readable medium, such as a disk (floppy diskette or hard disk), optical CD such as CD- or DVD-ROM/RAM, magnetic tape, electrical storage media such as RAM and ROM and hybrids of these such as magnetic/optical storage media.

Invention kits can additionally include a buffering agent, a preservative, or a protein/nucleic acid stabilizing agent. The kit can also include control components for assaying for activity, e.g., a control sample or a standard. Each component of the kit can be enclosed within an individual container or in a mixture and all of the various containers can be within single or multiple packages.

Antibodies (e.g., PM-2 antibody, produced by a cell line DSMZ Deposit No. DSM ACC 2600, or represented by heavy and light chain sequences set forth as SEQ ID NOs:1 and 2) nucleic acids, and other compositions and methods of the invention can be included in or employ pharmaceutical formulations. Such pharmaceutical formulations are useful for treatment of, or administration or delivery to, a subject in vivo or ex vivo.

Pharmaceutical formulations include “pharmaceutically acceptable” and “physiologically acceptable” carriers, diluents or excipients. As used herein the terms “pharmaceutically acceptable” and “physiologically acceptable” include solvents (aqueous or non-aqueous), solutions, emulsions, dispersion media, coatings, isotonic and absorption promoting or delaying agents, compatible with pharmaceutical administration. Such formulations can be contained in a liquid; emulsion, suspension, syrup or elixir, or solid form; tablet (coated or uncoated), capsule (hard or soft), powder, granule, crystal, or microbead. Supplementary compounds (e.g., preservatives, antibacterial, antiviral and antifungal agents) can also be incorporated into the formulations.

Pharmaceutical formulations can be made to be compatible with a particular local, regional or systemic administration or delivery route. Thus, pharmaceutical formulations include carriers, diluents, or excipients suitable for administration by particular routes. Specific non-limiting examples of routes of administration for compositions of the invention are parenteral, e.g., intravenous, intraarterial, intradermal, intramuscular, subcutaneous, intra-pleural, transdermal (topical), transmucosal, intra-cranial, intra-spinal, intra-ocular, rectal, oral (alimentary), mucosal administration, and any other formulation suitable for the treatment method or administration protocol.

Solutions or suspensions used for parenteral application can include: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide.

Pharmaceutical formulations for injection include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (for example, glycerol, propylene glycol, and liquid polyethylene glycol, and the like), and suitable mixtures thereof. Fluidity can be maintained, for example, by the use of a coating such as lecithin, by the maintenance of the required particle size in the case of dispersion and by the use of surfactants. Antibacterial and antifungal agents include, for example, parabens, chlorobutanol, phenol, ascorbic acid and thimerosal. Isotonic agents, for example, sugars, polyalcohols such as mannitol, sorbitol, sodium chloride can be included in the composition. Including an agent which delays absorption, for example, aluminum monostearate or gelatin can prolong absorption of injectable compositions.

Sterile injectable formulations can be prepared by incorporating the active composition in the required amount in an appropriate solvent with one or a combination of above ingredients. Generally, dispersions are prepared by incorporating the active composition into a sterile vehicle containing a basic dispersion medium and any other ingredient. In the case of sterile powders for the preparation of sterile injectable solutions, methods of preparation include, for example, vacuum drying and freeze-drying which yields a powder of the active ingredient plus any additional desired ingredient from a previously prepared solution thereof.

For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays, inhalation devices (e.g., aspirators) or suppositories. For transdermal administration, the active compounds are formulated into ointments, salves, gels, creams or patches.

The pharmaceutical formulations can be prepared with carriers that protect against rapid elimination from the body, such as a controlled release formulation or a time delay material such as glyceryl monostearate or glyceryl stearate. The formulations can also be delivered using articles of manufacture such as implants and microencapsulated delivery systems to achieve local, regional or systemic delivery or controlled or sustained release.

Biodegradable, biocompatible polymers can be used, such as ethylene vinyl acetate, polyanhydrides, polyglycolic acid, collagen, polyorthoesters, and polylactic acid. Methods for preparation of such formulations are known to those skilled in the art. The materials can also be obtained commercially from Alza Corporation (Palo Alto, Calif.). Liposomal suspensions (including liposomes targeted to cells or tissues using antibodies or viral coat proteins) can also be used as pharmaceutically acceptable carriers. These can be prepared according to known methods, for example, as described in U.S. Pat. No. 4,522,811.

Additional pharmaceutical formulations appropriate for administration are known in the art (see, e.g., Gennaro (ed.), Remington: The Science and Practice of Pharmacy, 20^{th} ed., Lippincott, Williams & Wilkins (2000); Ansel et al., Pharmaceutical Dosage Forms and Drug Delivery Systems, 7^{th} ed., Lippincott Williams & Wilkins Publishers (1999); Kibbe (ed.), Handbook of Pharmaceutical Excipients American Pharmaceutical Association, 3^{rd} ed. (2000); and Remington's Pharmaceutical Principles of Solid Dosage Forms, Technonic Publishing Co., Inc., Lancaster, Pa., (1993)).

The compositions used in accordance with the invention, including proteins (antibodies), nucleic acid (inhibitory), treatments, therapies, agents, drugs and pharmaceutical formulations can be packaged in dosage unit form for ease of administration and uniformity of dosage. “Dosage unit form” as used herein refers to physically discrete units suited as unitary dosages treatment; each unit contains a quantity of the composition in association with the carrier, excipient, diluent, or vehicle calculated to produce the desired treatment or therapeutic (e.g., beneficial) effect. The unit dosage forms will depend on a variety of factors including, but not necessarily limited to, the particular composition employed, the effect to be achieved, and the pharmacodynamics and pharmacogenomics of the subject to be treated.

The invention provides cell-free (e.g., in solution, in solid phase) and cell-based (e.g., in vitro or in vivo) methods of screening, detecting and identifying a cell, antigen or epitope to which PM-2 antibody, produced by a cell line DSMZ Deposit No. DSM ACC 2600, or represented by heavy and light chain sequences set forth as SEQ ID NOs:1 and 2, binds. The methods can be performed in solution, in vitro using a biological material or sample, and in vivo, for example, using neoplastic, tumor or cancer, or metastasis cells, tissue or organ (e.g., a biopsy) from an animal.

In accordance with the invention, there are provided methods of identifying, detecting or screening for a cell, antigen or epitope to which PM-2 antibody, produced by a cell line DSMZ Deposit No. DSM ACC 2600, or represented by heavy and light chain sequences set forth as SEQ ID NOs:1 and 2, binds. In one embodiment, a method includes contacting a biological material or sample with a PM-2 antibody, produced by a cell line DSMZ Deposit No. DSM ACC 2600, or represented by heavy and light chain sequences set forth as SEQ ID NOs:1 and 2, binds under conditions allowing binding of the antibody to a cell, antigen or epitope; and assaying for binding of the antibody to the cell, antigen or epitope. The binding of the antibody to a cell, antigen or epitope detects their presence. In one aspect, the biological material or sample is obtained from a mammalian subject. In a further aspect, the antibody that binds to the cell, antigen or epitope is distinct from PM-2 antibody, produced by a cell line DSMZ Deposit No. DSM ACC 2600, or represented by heavy and light chain sequences set forth as SEQ ID NOs:1 and 2, binds.

The invention also provides cell-free (e.g., in solution, in solid phase) and cell-based (e.g., in vitro or in vivo) methods of diagnosing and monitoring progression of a subject having or at increased risk of having undesirable or aberrant cell proliferation or a cellular hyperproliferative disorder (e.g., neoplasia, tumor or cancer, or metastasis). The methods can be performed in solution, in vitro using a biological material or sample, for example, a biopsy of suspicious cells that may comprise or be indicative of neoplastic, tumor or cancer, or metastasis cells, tissue or organ. The methods can also be preformed in vivo, for example, in an animal.

In accordance with the invention, there are provided methods of diagnosing and monitoring progression of a subject having or at increased risk of having undesirable or aberrant cell proliferation or a cellular hyperproliferative disorder (e.g., neoplasia, tumor or cancer, or metastasis). In one embodiment, a method includes providing a biological material or sample from a subject, contacting the biological material or sample with a PM-2 antibody, produced by a cell line DSMZ Deposit No. DSM ACC 2600, or represented by heavy and light chain sequences set forth as SEQ ID NOs:1 and 2, under conditions allowing binding of the antibody to a cell, antigen or epitope; and assaying for binding of the antibody to the cell, antigen or epitope. The binding of the antibody to the cell, antigen or epitope diagnoses the subject as having or at increased risk of having undesirable or aberrant cell proliferation or a cellular hyperproliferative disorder (e.g., neoplasia, tumor or cancer, or metastasis). In one aspect, the biological material or sample is obtained from a human. In another aspect, the biological material or sample comprises a biopsy (e.g., a biopsy of lung, pancreas, stomach, breast, esophagus, ovary or uterus). Methods of monitoring progression of undesirable or aberrant cell proliferation or a cellular hyperproliferative disorder (e.g., neoplasia, tumor or cancer, or metastasis) can be performed at a regular or irregular intervals, for example, daily, bi-weekly, weekly, bimonthly, monthly, quarterly, semi- or bi-annually, annually, etc., as appropriate.

Identifying, detecting, screening and diagnostic assays of the invention can be practiced by analysis of suspect hyperproliferating cells, for example, a cell of a cellular hyperproliferative disorder or an appropriate sample. Cells include hyperproliferating, immortalized, neoplastic, tumor and cancer cell lines and primary isolates derived from breast, lung, thyroid, head and neck, nasopharynx, nose or sinuses, brain, spine, adrenal gland, thyroid, lymph, gastrointestinal (mouth, esophagus, stomach, duodenum, ileum, jejunum (small intestine), colon, rectum), genito-urinary tract (uterus, ovary, endometrium, cervix, bladder, testicle, penis, prostate), kidney, pancreas, adrenal gland, liver, bone, bone marrow, lymph, blood, muscle, skin, and the hematopoetic system, and metastasis or secondary sites.

The term “contacting,” when used in reference to a composition such as a protein (e.g., antibody), material, sample, or treatment, means a direct or indirect interaction between the composition (e.g., protein such as an antibody) and the other referenced entity. A particular example of direct interaction is binding. A particular example of an indirect interaction is where the composition acts upon an intermediary molecule, which in turn acts upon the referenced entity. Thus, for example, contacting a cell (e.g., that comprises a cellular hyperproliferative disorder) with an antibody includes allowing the antibody to bind to the cell, or allowing the antibody to act upon an intermediary (e.g., antigen) that in turn acts upon the cell.

The terms “assaying” and “measuring” and grammatical variations thereof are used interchangeably herein and refer to either qualitative or quantitative determinations, or both qualitative and quantitative determinations. When the terms are used in reference to binding, any means of assessing the relative amount, affinity or specificity of binding is contemplated, including the various methods set forth herein and known in the art. For example, antibody binding can be assayed or measured by an ELISA assay, Western blot or immunoprecipitation assay.

Unless otherwise defined, all technical and scientific terms used herein have the same meaning as commonly understood by one of ordinary skill in the art to which this invention relates. Although methods and materials similar or equivalent to those described herein can be used in the practice or testing of the invention, suitable methods and materials are described herein.

All publications, patents, Genbank accession numbers and other references cited herein are incorporated by reference in their entirety. In case of conflict, the present specification, including definitions, will control.

As used herein, singular forms “a”, “and,” and “the” include plural referents unless the context clearly indicates otherwise. Thus, for example, reference to an “antibody” includes a plurality of antibodies and reference to “a treatment or therapy” can include multiple simultaneous, consecutive or sequential doses, treatments or therapies, and so forth.

As used herein, all numerical values or numerical ranges include whole integers within or encompassing such ranges and fractions of the values or the integers within or encompassing ranges unless the context clearly indicates otherwise. Thus, for example, reference to a range of 90-100%, includes any numerical value or range within or encompassing such values, such as 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and any numerical range within such a range, such as 90-92%, 90-95%, 95-98%, 96-98%, 99-100%, etc. In an additional example, reference to a range of 1-5,000 fold includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and any numerical range within such a range, such as 1-2,5-10, 10-50, 50-100, 100-500, 100-1000, 500-1000, 1000-2000, 1000-5000, etc. In a further example, reference to a range of KD 10⁻⁵ M to about KD 10⁻¹³ M includes any numerical value or range within or encompassing such values.

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed.

A number of embodiments of the invention have been described. Nevertheless, it will be understood that various modifications may be made without departing from the spirit and scope of the invention. Accordingly, the following examples are intended to illustrate but not limit the scope of invention described in the claims.

### EXAMPLES

### Example 1

This example includes a description of various materials and methods.

### Cell Culture

The following human cell lines were used: BXPC-3 (pancreatic adenocarcinoma; ATCC (American Type Culture Collection, P.O. Box 1549, Manassas, Va. 20108) Accession No. CRL-1687), CACO-2 (colon adenocarcinoma), Colo-206F (colon carcinoma) and or A549 (lung). The cell lines were cultured in RPM1-1640 media (PAA, Vienna, Austria) supplemented with 10% fetal calf serum (FCS), 2 mM glutamine and penicillin/streptomycin (both 1%) and incubated in a humidified, 5% CO₂ atmosphere at 37° C. For the assays described, cells were grown to sub-confluency, detached with trypsin/EDTA and washed twice with phosphate-buffered saline (PBS) before use.

### Producing Hybridomas

Lymphocytes were immortalized by fusing them to the HAB-1 heteromyeloma as follows: HAB-1 heteromyeloma cells were washed twice with RPMI 1640 (PAA, Vienna, Austria) without additives and centrifuged the cells for 5 minutes at 1500 rpm. We then thawed frozen lymphocytes obtained from either the spleen or the lymph nodes and we washed these cells twice with RPMI 1640 without additives and centrifuged these cells at 1500 rpm for 5 minutes. Both the HAB-1 and the lymphocyte cell pellets were resuspended in 10 ml RPMI 1640 without additives and were counted in a Neubauer cell counting chamber. We washed the cells again, added the HAB-1 cells and the lymphocytes together in a ratio of 1:2 to 1:3, mixed them, and centrifuged the mixture for 8 minutes at 1500 rpm. We pre-warmed Polyethylene Glycol 1500 (PEG) to 37° C. and carefully let the PEG run drop-wise onto the pellet while slightly rotating the 50 ml tube. Next, we gently resuspended the pellet and rotated the tube for exactly 90 seconds in a 37° C. water bath. We washed the cells twice with a full 10 ml pipette of RPMI without additives and centrifuged the cells for 5 minutes at 1500 rpm. We added 1 ml of RPMI 1640 with HAT supplement (PAA, Vienna, Austria) and 10% FCS, 1% glutamine, and 1% penicillin/streptomycin (“RPMI 1640 HAT”) into each well of a 24-well plate.

The cell pellet was dissolved in RPMI 1640 HAT and 0.5 ml of the cells was added to each well of the 24-well plate. We then placed the 24-well plates into a 37° C. incubator and changed the RPMI 1640 HAT medium weekly. After four to six weeks, the cell culture supernatants were screened for antibody production in an enzyme-linked immunosorbent assay (ELISA).

Using this protocol, approximately 80% to 90% of the triomas generated are viable and approximately 50% secrete immunoglobulins. Positive clones were tested immunohistochemically on autologous tumor tissue sections and clones that showed a positive reaction were subsequently re-cloned.

cDNA Synthesis and RT-PCR

To obtain the sequence of the antibody, we isolated whole RNA from the trioma using the RNASE Kit from Qiagen. Total RNA may also be prepared using methods standard in the art, e.g., those described in Krenn et al. (Clin. Exp. Immunol. 115:168-175, 1999). cDNA synthesis from total RNA obtained from hybridoma cell line PM-2 was performed with 5 μg total RNA using Gibco BRL (Eggenstein, Germany) M-MLV Reverse Transcriptase according to the manufacturer's instructions. The amplification of V_{H} and V_{L} genes was carried out in a 25 μl volume with 1.75 mM MgCl₂, 0.4 μM primer, 200 μM of each dNTP, and 1 U Taq polymerase (MBI Fermentas, St. Leon-Rot, Germany). The PCR-products were amplified using the following cycle profiles: 95° C. for 2 min, followed by 35 cycles of 94° C. for 30 sec; 65° C. for 30 sec (for VH3 and VH4 primers), 60° C. for VH1, VH2, VH5, VH6 and 52° C. for VL primers respectively; a final extension at 72° C. for 4 min.

### Sequencing the Antibody

The PCR products were purified using gel electrophoresis through 2% agarose (Roth, Karlsruhe, Germany) followed by gel extraction of the PCR product using a Jetsorb gel extraction kit (Genomed, Bad Oeynhausen, Germany). The PCR product were then cloned using the pCR-Script Amp SK⁺ cloning kit (Stratagene, Heidelberg, Germany). Ten positive clones were sequenced using the DyeDeoxy termination cycle sequencing kit (Applied BioSystems Inc., Weiterstadt, Germany) and analysed with an ABIPrism373 automated DNA sequencer (both strands were sequenced using T3 and T7 primers). The sequences were analysed using the DNASIS for Windows sequence comparison software and the GenBank and IMGT/V-QUEST databases. The International Immunogenetics (“IMGT”) database is coordinated by Marie-Paule Lefranc at the Université Montpellier, Montpellier, France.

### Immunohistochemical Staining of Paraffin Sections

Paraffin-embedded human tissues were sectioned (2 μm), and the paraffin was removed as follows: Two xylene washes for 5 minutes each, Two 100% ethanol washes for 5 minutes each, Two 90% ethanol washes for 5 minutes each, Two 70% ethanol washes for 5 minutes each, and Three washes in distilled H₂O.

The slides containing the tissue sections were incubated in 75 ml distilled H₂O and 25 ml de-masking solution (Demaskierungslösung G, Biologo, Kronshagen, Germany) in a preheated water-bath at 100° C. for 20 minutes. The slides were placed into Tris/NaCl (3 grams Tris, 40.5 grams NaCl in 5 litres of distilled H₂O and pH adjusted to 7.4 with HCl) for 5 minutes, blocked for 15-30 minutes with 15 μl of 0.5% Bovine Serum Albumin Fraction V (“BSA;” Roth, Karlsruhe, Germany) in phosphate buffered saline (“PBS”) per slide, and washed once with Tris/NaCl.

The sections were incubated with PM-2 antibody, and unrelated, human monoclonal IgM antibodies (ChromPure IgM, Dianova, Hamburg, Germany, 10 μg/ml) or mouse CAM 5.2 antibody diluted 1:50 with BSA/PBS (Dako, Hamburg, Germany) for 2.5 hours in a humidified incubator at 37° C. The sections were then washed three times with Tris/NaCl (3 grams Tris, 140.5 grams NaCl in 5 litres of distilled H₂O and pH adjusted to 7.4 with HCl), followed by incubation with peroxidase-labeled rabbit anti-human or rabbit anti-mouse conjugate (Dako) diluted 1:50 in PBS containing 30% rabbit serum (for antibody 103/51) at RT for 1 hour. After washing three times with Tris/NaCl the tissue sections were incubated in PBS for 10 minutes before staining with diaminobenzidine (0.05%)-hydrogen peroxide (0.02%) for 10 minutes at room temperature (RT). The reaction was stopped using running tap water and the sections counterstained with hematoxylin. After mounting with glycerol-gelatin, the sections were analyzed using light microscopy.

Immunohistochemical Staining of Cryo-Sections from Autologous Tumors

Frozen human tissues were sectioned (4 μm), fixed in acetone, air-dried and washed with Tris/NaCl (3 grams Tris, 40.5 grams NaCl in 5 litres of distilled H₂O and pH adjusted to 7.4 with HCl). The cryo-sections were then blocked with PBS containing 3% milk powder for 30 minutes at RT. After washing three times with Tris/NaCl the sections were incubated with PM-2 human IgM antibodies, unrelated human monoclonal IgM (Chrompure IgM, Dianova, 10 μg/ml) or mouse CAM 5.2 antibody diluted 1:50 with BSA/PBS (Dako) for 30 minutes at RT. The sections were washed three times with Tris/NaCl, followed by incubation with secondary antibodies (peroxidase-labeled rabbit anti-human or rabbit anti-mouse conjugate 1:50) for 30 minutes at RT. After washing three times with Tris/NaCl and incubation in PBS for 10 minutes, the sections were stained with diaminobenzidine (0.05%)-hydrogen peroxide (0.02%) for 10 minutes at RT. The reaction was stopped under running tap water and the sections counterstained with hematoxylin. After mounting with glycerol-gelatin, the sections were analyzed using light microscopy.

### Preparation of Tumor Cell Membrane Extracts

Isolation of membrane proteins from tumor cells was performed as described using standard methods in the art, as described, for example, in Hensel et al. (Int. J. Cancer 81:229-235, 1999). In particular, confluent BXPC-3 tumor cells were washed twice with PBS, harvested with a cell scraper, centrifuged, and resuspended in hypotonic buffer (20 mM HEPES, 3 mM KCl, 3 mM MgCl₂) and incubated for 15 minutes on ice. The cells were then sonicated for 5 minutes and the nuclei were pelleted by centrifugation at 10,000×g for 10 min. The supernatant was centrifuged for 40 minutes at 100,000×g in a swing-out rotor to pellet the membranes. After washing the pellet with hypotonic buffer, the pellet was resuspended in membrane lysis buffer (50 mM HEPES pH 7.4, 0.1 mM EDTA, 10% glycerol, and 1% Triton X-100). Complete protease inhibitor (Boehringer, Mannheim, Germany) also was added to all solutions.

### Western Blotting

Western blots were preformed using standard techniques as described, for example, in Hensel et al. (Int. J. Cancer 81:229-235, 1999). In short, blotted nitrocellulose membranes were blocked with PBS containing 3% low fat milk powder, followed by incubation for 1 hour with 20-40 μg of PM-2 human IgM antibodies or unrelated human control IgM (ChromPure IgM, Dianova). The secondary antibody (peroxidase-coupled rabbit anti-human IgM antibody 1:1,000, Dianova) was detected with the SUPERSIGNAL chemiluminescence kit from Pierce (KMF, St. Augustin, Germany).

### Cytospin Preparation

The adherent growing cells were detached by adding Trypsin/EDTA (PAA, Vienna, Austria) followed by a 5 minute incubation in an humidified incubator (37° C., 5% CO₂) and centrifugation for 5 minutes at 1,500 rpm. The cells then were washed twice with 10 ml of RPMI-1640 cell culture medium (PAA, Vienna, Austria). The cell number was adjusted to a density of 1×10⁵ cells/ml. From this solution, 100 μl were centrifuged onto microscope slides with a cytospin centrifuge (CYTOSPIN 2, Shandon, UK) for 2 minutes at 50 rpm. The resultant cytospins were dried for at least 2 hours and stained as specified below.

### Immunoperoxidase Staining of Cytospins and Cryosections

Cytospins were dried for at least two hours at room temperature or cryosections were dried for at least two hours after they were cut. The sections or cytospins were then fixed for 10 minutes in acetone. The fixed cryosections/cytospins were dried for 30 minutes at room temperature, washed three times with Tris-NaCl (3 grams Tris, 40.5 grams NaCl in 5 litres of distilled H₂O and pH adjusted to 7.4 with HCl), and placed into Tris/NaCl for 5 minutes. The cryosections/cytospins were blocked for 15-30 minutes with 3% milk powder in PBS (100 μl per cryosection/cytospin) and washed three times with Tris-NaCl. The cryosections/cytospins were incubated in 100 μl of primary antibody per cryosection/cytospin (e.g., at 20 μg/ml in 0.5% BSA/PBS; CK 8 at 1:50 in BSA/PBS; CAM 5.2 at 1:10 in BSA/PBS; or RPMI 1640 media (PAA, Vienna, Austria) as a negative control) for 30 minutes in a humidified chamber at room temperature. Following the incubation, the cryosections/cytospins were washed three times with Tris-NaCl.

The cryosections/cytospins were then incubated in 100 μl of a solution containing the secondary antibody (70% PBS+30% rabbit or human serum+e.g., 1:50 rabbit anti-mouse antibody, peroxidase coupled or 1:50 rabbit anti-human IgM antibody, peroxidase coupled; Dako, Hamburg, Germany) per cryosection/cytospin for 30 minutes in a humidified chamber at room temperature and washed three times with Tris-NaCl and placed into PBS for 10 minutes. The cryosections/cytospins where then incubated for 10 minutes in 100 μl of a solution containing 0.05% diaminobenzidine and 0.02% hydrogen peroxide (Sigma, Taufkirchen (München), Germany). Following the incubation, the cryosections/cytospins were washed with distilled H₂O and placed into a hematoxylin staining solution (Roth, Karlsruhe, Germany) for 5 minutes. The cryosections/cytospins were then rinsed for 15 minutes under running tap water, washed with distilled H₂O, and cover with pre-warmed glycerol-gelatin.

### Example 2

This example includes a description of the generation of the cell line expressing PM-2 monoclonal antibody.

As described above, we obtained the PM-2 monoclonal antibody expressing hybridoma by fusing lymphocytes obtained from the spleen or lymph nodes of a cancer patient with the heteromyeloma cell line HAB-1 (Faller, et al., Br. J. Cancer 62:595-598, 1990). The lymphoid sources were not pre-selected in terms of the age or sex of the patient. The resultant cell is a type of hybridoma known as a trioma, as it is the fusion of three cells. Like normal B-lymphocytes, this trioma has to ability to produce antibodies. The specificity of the antibody is determined by the specificity of the original lymphocyte from the patient that was used to generate the trioma.

The hybridoma supernatants were screened for antibody production using an ELISA assay. Following ELISA, antibodies were primarily tested immunohistochemically against their autologous tumor for tumor specific reactivity. PM-2 was generated from the lymphocytes of a pancreatic cancer patient.

The amino acid sequence (SEQ ID NO: 1) and the nucleic acid sequence (SEQ ID NO:3) of the variable region of the heavy chain of human monoclonal antibody PM-2 are disclosed herein. CDR1 of the PM-2 variable region heavy chain spans nucleotides 31-54 which encode amino acids 11-18, CDR2 spans nucleotides 106-129 which encode amino acids 36-43, and CDR3 spans nucleotides 244-300, which encode amino acids 82-100.

The amino acid sequence (SEQ ID NO:2) and the nucleic acid sequence (SEQ ID NO:4) of the variable region of the light chain of human monoclonal antibody PM-2 are disclosed herein. CDR1 of the PM-2 variable region light chain spans nucleotides 76-102 which encode amino acids 26-34, CDR2 spans nucleotides 154-174 which encode amino acids 52-58, and CDR3 spans nucleotides 289-309, which encode amino acids 97-103.

### Example 3

This example includes a description of immunohistochemical characterization of PM-2 antibody.

To characterize the PM-2 monoclonal antibody secreted by a hybridoma, we tested the antibody against a panel of normal and tumor tissues using an immunoperoxidase assay as described in the materials and methods. This assay provided us with an overview of which tissues were stained by the antibody and of the distribution of the antigen.

First, we tested antibody against the same types of tumors from different patients. We then tested antibodies against tumors of other organs and, finally, against normal tissues. Using these assays, we identified the human PM-2 monoclonal antibody. The PM-2 monoclonal antibody is of the IgM/λ isotype (Table 1).

TABLE 1
Origin of Monoclonal IgM Antibodies and Clinical Data of Cancer Patients

| | | | Tumour | Tumour | | | Source of | Ig |
|---|---|---|---|---|---|---|---|---|
| Antibody | Organ | Tumour type | stage | grade | Age | Sex | Lymphocytes | Class |
| PM-2 | Pancreas | Adenocarcinoma | T1N1 | G2-3 | 47 | M | Spleen | IgM/λ |
| | | | | | | | | IgM/λ |

To investigate the genetic origin of the human monoclonal IgM antibody the V_{H} and V_{L} genes were amplified, cloned and sequenced. The sequences were compared with germ-line sequences in the IMGT/V-QUEST database to identify the most homologous germ-line genes and to detect somatic mutations. The results are represented in Table 2. The degree of identity of the nucleotide sequences of the V_{H} segment to those of the closest reported germ-line V_{H} genes ranged from 97.2 to 100% as summarized in Table 2.

TABLE 2
Characterization of Variable Heavy and Light Chain Regions of Monoclonal IgM Antibodies

| | Heavy chain | | | | Light chain | | | |
|---|---|---|---|---|---|---|---|---|
| | Germ- | | | | Germ- | | | |
| | line | Homology | R/S | R/S | line | Homology | R/S | R/S |
| Antibody | gene | (%) | Frame | CDR | gene | (%) | Frame | CDR |
| PM-2 | IGHV3-23*01 | 100 | 0/0 | 0/0 | IGLV5-45*01 | 98.2 | 3/2 | 0/0 |

The high homology of the VH region to the germ-line gene and the low R/S ratio, which is an indicator for affinity maturation of antibodies, indicates that the antibody did not undergo affinity maturation by somatic mutation due to antigen contact. The degree of identity of the nucleotide sequence of the V_{L} segment to their most homologous V_{L} germ-line gene ranged from 97.2 to 99.0%, utilizing a λ-light chain gene. The data indicate that PM-2 belongs to the family of naturally occurring, non-affinity matured antibodies.

After initial testing on autologous tumors, the reaction pattern of PM-2 antibody was investigated in greater detail using immunohistochemical staining on a variety of paraffin- and cryo-embedded carcinomas and normal tissues. The PM-2 antibody exhibited no detectable binding to normal tissues (Table 3).

TABLE 3
Reaction Pattern of the Monoclonal IgM PM-2 on Normal Tissues

| Tissue | PM-2 |
|---|---|
| Stomach | − |
| Colon | − |
| Lung | − |
| Esophagus | − |
| Urinary bladder | − |
| Prostate | − |
| Breast | − |
| Pancreas | − |
| Small Intestine | − |

Antibody PM-2 gave a broad staining pattern on a variety of tumor tissues that were tested including adenocarcinoma of pancreas and invasive ductal carcinoma of breast. PM-2 antibody binds to a large number (99%) of the 147 different tumor samples (carcinomas) screened (Table 4).

TABLE 4
Reaction Pattern of Monoclonal IgM
Antibody PM-2 on Tumor Tissues

| | Carcinoma | | | IgM- |
|---|---|---|---|---|
| Tissue | type | PM-2 +/− | CAM5.2 | Control |
| Stomach | Adeno | 26/1 | + | − |
| Colon | Adeno | 3/0 | + | − |
| Small Intestine | | 1/0 | + | − |
| Lung | Adeno | 31/0 | + | − |
| | Squamous cell | 31/0 | +(CK5/6) | |
| Liver | | 2/0 | + | − |
| Esophagus | Squamous cell | 3/0 | +(CK5/6) | − |
| Pancreas | Adeno | 27/0 | + | − |
| Urinary bladder | Urothel | 1/0 | + | − |
| Kidney | Renal cell | 1/0 | − | − |
| | Adeno | n.d. | | − |
| Prostate | Adeno | 4/1 | + | − |
| Breast | Invasive | 3/0 | + | − |
| | (ductal) | | | |
| | Invasive | 3/0 | + | − |
| | (lobular) | | | |
| Ovary | Adeno | 3/0 | + | − |
| Uterus | Adeno | 3/0 | + | − |
| Adrenal Gland | Adeno | 1/0 | + | − |

The positive control antibody in these experiments was a mouse monoclonal antibody against human cytokeratin 5/6 (“CK 5/6;” Dako A/S, Denmark) or a mouse monoclonal antibody against human cytokeratin (“CAM 5.2;” Becton Dickinson, N.J.).

To determine the antigen recognized by PM-2, western blots were performed with membrane extracts of pancreas adenocarcinoma cell line BXPC-3. Antibody PM-2 reacted with protein(s) antigen of about 115 kDa on cell line BXPC-3. Antibody PM-2 reacted with the protein antigen on MKN and CRL cell membrane fractions, which sometimes appears as a double band of about 100 and 115 kDa.

PM-2 also specifically stained a number of carcinoma cell lines. In particular, PM-2 antibody specifically binds to the CACO-2 human colorectal adenocarcinoma cell line (ATCC Accession No. HBT-37, DSMZ Accession No. ACC 169), the human colon carcinoma cell line COLO-320 (DSMZ Accession No. ACC 144), the human colon carcinoma cell line COLO-206F (DSMZ Accession No. ACC 21), the HT-29 human colorectal adenocarcinoma cell line (ATCC Accession No. HTB-38), ASPC-1 pancreatic carcinoma cells, BXPC-3 pancreatic carcinoma cell line and A549 lung carcinoma cells.

### Example 4

This example includes a description of PM-2 antibody inducing apoptosis. A

A number of assays standard in the art may be used to determine if an antibody induces apoptosis of a cell. For example, the CELL DEATH DETECTION ELISA^{PLUS} (Roche, Mannheim, Germany) was used to analyze the extent to which PM-2 antibody induces apoptosis. The cell death detection ELISA is based on a quantitative sandwich-enzyme-immunoassay principle using mouse monoclonal antibodies directed against DNA and histones, respectively. This assay allows the specific determination of mono- and oligo-nucleosomes which are released into the cytoplasm of cells which die from apoptosis.

In particular, 1×10⁴ BXPC-3 tumor cells were plated on 96-well plates and incubated in presence of different concentrations of the human IgM-antibodies for 24 hours at 37° C. and 7% C0₂ in an C0₂ incubator. Depleted cell culture supernatant with unrelated IgM antibodies served as negative control. After the incubation period, cells were centrifuged for 10 minutes and the supernatants were removed. The resulting cell pellets were then incubated with lysis-buffer for 30 minutes at room temperature. After centrifugation the supernatants were transferred into a streptavidin-coated microtiter plate (MTP) and immunoreagent (a mixture of 10% Anti-Histone-Biotin, 10% Anti-DNA-peroxidase (Anti-DNA POD) and 80% incubation buffer) added before incubation for 2 hours at room temperature on a MTP shaker at 250 rpm. Following the incubation period, unbound components were removed by a washing step with incubation buffer. POD was determined photometrically with ABTS™ as a substrate (1 ABTS™ (2,2′-Azino-di[3-ethyl-benz-thiazolin-sulfonat) tablet in 5 ml substrate buffer). Antibody-induced apoptosis was measured by determining the color intensity of the green precipitate that it formed as a result of this reaction using an ELISA reader at a wavelength of 415 nm in comparison to ABTS™ solution as a blank (reference wavelength of approximately 490 nm). Based on this color intensity, we calculated the level of the antibody-induced apoptosis. These experiments clearly showed that PM-2 induces apoptosis in BXPC-3 pancreas tumor cell line after 24 hours of incubation.

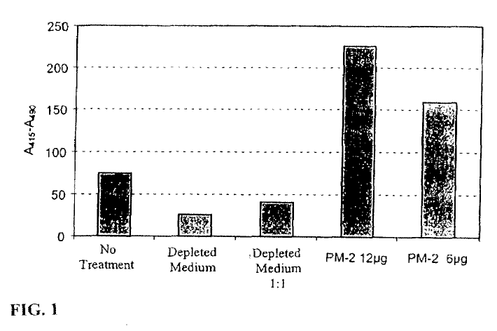

In addition, as shown in FIG. 1, PM-2 monoclonal antibody induces apoptosis in BXPC-3 human pancreatic carcinoma cells after a 24 hour incubation period when compared to a negative control. The Y-axis is the difference between the absorbance at 415 nm and at the 490 nm reference wavelength (A₄₁₅-A₄₉₀) and the negative control is RPMI 1460 medium. The concentration of the PM-2 antibody was either 6 μg or 12 μg/ml in supernatant.

### Example 5

This example includes a description of antibody inhibiting cell proliferation.

Cell proliferation may be assayed by a number of methods that are standard in the art, for example, by the reduction of tetrazolium salts. The yellow tetrazolium salt 3-(4,5-dimethylthiazol-2-yl)-2,5-diphenyltetrazolium bromide (“MTT”) (Sigma, St. Louis, Mo.), is reduced by metabolically active cells, in part by the action of mitochondrial dehydrogenase enzymes to generate reducing equivalents such as NADH and NADPH. The resulting intracellular purple formazan can be solubilized and quantified by spectrophotometric means. The MTT cell proliferation assay measures the rate of cell proliferation and, when metabolic events lead to apoptosis, the reduction in cell viability.

For the MTT assay, we trypsinized BXPC-3 cells and resuspended the cells in 10 ml of RPMI-1460 medium contains 10% Fetal Calf Serum (FCS), 1% glutamine, and 1% penicillin/streptomycin (complete medium). The cells were then counted and diluted to 1×10⁶ cells/ml. 50 μl of this suspension were pipetted into wells of a 96-well plate, resulting in approximately 5×10⁴ cells/well. The first row of wells was left empty. We then added 50 μl of the antibody diluted in complete medium to each well. The 96-well plate was then incubated for 24 or 48 hours in a 37° C. incubator. After the incubation period, 50 μl MTT solution (5 mg/ml in PBS) were added to each well. The 96-well plate was incubated for 30 minutes at 37° C. and centrifuged for 5 minutes at 800×g. The supernatant was aspirated, 150 μl of dimethylsulphoxide (DMSO) were added to each well, and the cell pellet was resuspended. Absorption was determined at a wavelength of 540 nm and at a reference wavelength of 690 nm in an ELISA reader.

After 24 or 48 hours, PM-2 antibody inhibited cell proliferation of the respective tumor cell lines in a concentration-dependent manner, while the controls with depleted cell culture supernatant remained unchanged.

Similarly, incubation of BXPC-3 pancreatic carcinoma cells with PM-2 monoclonal antibody resulted in a decrease in proliferation and cell viability after both a 24 hour and a 48 hour period.

### Example 6

This example includes a description of in vivo imaging of a neoplasm.

A patient suspected of having a neoplasm, such as a colorectal carcinoma, may be given a dose of radioiodinated PM-2 antibody, or another tumor-specific polypeptide, and radiolabeled unspecific antibody using the methods described herein. Localization of the tumor for imaging may be effected according to the procedure of Goldenberg et al. (N. Engl. J. Med., 298:1384, 1978). By I.V. an infusion of equal volumes of solutions of ¹³¹I-PM-2 antibody and Tc-99m-labeled unspecific antibody may be administered to a patient. Prior to administration of the reagents I.V., the patient is typically pre-tested for hypersensitivity to the antibody preparation (unlabeled) or to antibody of the same species as the antibody preparation. To block thyroid uptake of ¹³¹I, Lugol's solution is administered orally, beginning one or more days before injection of the radioiodinated antibody, at a dose of 5 drops twice or three-times daily. Images of various body regions and views may be taken at 4, 8, and 24 hours after injection of the labeled preparations. If present, the neoplasm, e.g., a colorectal carcinoma, is detected by gamma camera imaging with subtraction of the Tc-99m counts from those of ¹³¹I, as described for ¹³¹I-labeled anti-CEA antibody and Tc-99m-labeled human serum albumin by DeLand et al. (Cancer Res. 40:3046, 1980). At 8 hours after injection, imaging is usually clear and improves with time up to the 24 hour scans.

### Example 7

This example includes a description of studies of disseminated tumor cells (DTC).

Detection of disseminated tumor cells (DTC) in the blood is dependent upon many variables including shedding of cells from the tumor, preparing for sampling, and time point of sampling. Thus, positive or negative results in blood of small animals may be variable.

Detection of DTC in bone marrow may be a more accurate indicator of DTC cells in animals, because bone marrow is a much more “steady” compartment than blood. In addition, tumor cells in bone marrow are more locally fixed and once these cells have entered bone marrow are more likely to remain in the marrow. Thus, detection of DTC in bone marrow is less dependent upon the variables that affect measurement of DTC the blood.

To determine the effect of PM-2 antibody on DTC, a mouse animal model of xenotransplanted gastric cancer (a gastric adenocarcinoma cell line) was used (Illert et al., Clin. Exp. Metastasis 20:549 (2003)). In this animal model, when the gastric adenocarcinoma cell line is transplanted into stomach there is local tumor growth and formation of distant metastasis. DTC is also detected in bone marrow and in blood in this model. Development of DTC in this animal model correlated with metastasizing tumor growth (Illert et al., Clin. Exp. Metastasis 20:549 (2003)).

Expression of cytokeratin 20 (CK20) is a marker for DTC in this gastric cancer animal model. The presence of DTC in bone marrow and blood of animals with gastric cancer treated with antibody PM-2 or control IgM was determined by measuring CK20 levels. The data indicate that CK20 levels were reduced in animals treated with PM-2 antibody as compared to control IgM, indicating that PM-2 reduced DTC in animals.

DTC in bone marrow in animals treated with a 300 μg dosage, 5 times (20 animals per group): for IgM 14 animals were positive for DTC; whereas in the PM-2 treated group only 7 animals were positive for DTC. This data indicates a reduction of DTC in the bone marrow of PM-2 treated animals from 70% to 35% (p=0.0562; fisher's exact test).

As a measurement of total tumor load in animals, DTC was determined in bone marrow and in blood of animals. In the control IgM group 10 out of 20 animals had DTC in both bone marrow and blood; after PM-2 therapy only 3 of 20 animals had DTC in both bone marrow and blood (50% vs. 15%, p=0.0407, fisher's exact test).

Comparison of a higher frequency and dose (dosage 5×300 μg) of PM-2 with a lower frequency and dose (3×200 μg) shows a reduction of DTC in bone marrow with increased dose. At a lower frequency and dose (3×200 μg) 7/10 animals were positive for DTC (70%) in bone marrow. In contrast, at the higher frequency and dose (dosage 5×300 μg) 7/20 animals were positive for DTC (35%) in bone marrow.

The foregoing results indicate that PM-2 antibody can reduce the establishment and formation of metastasis.

### Example 8

This example includes a description of additional immunohistochemical characterization of PM-2 antibody.

Immunohistochemistry analysis revealed that PM-2 antibody binds to various forms of cancer. In particular, PM-2 binds to all grades and stages of lung adenocarcinoma, and no differences between males or females were detected. PM-2 binds to all grades and stages of lung squamous cell carcinoma, and no differences between males or females were detected. Thus, PM-2 antigen is therefore ubiquitously expressed on all grades and stages of lung adenocarcinoma and lung squamous cell carcinoma of both males and females. PM-2 antigen is therefore a target and PM-2 antibodies and functional fragments thereof a therapy for treating all stages of lung adenocarcinoma and lung squamous cell carcinoma in both males and females.

Immunohistochemistry analysis also revealed that PM-2 antibody binds to various metastatic forms of cancer. In particular, PM-2 binds to lymph node and brain metastasis arising from lung adenocarcinoma and lung squamous cell carcinoma. PM-2 also binds to lymph node metastasis arising from breast invasive ductal and invasive lobular cancer. PM-2 further binds to liver and lymph node metastasis arising from colon adenocarcinoma. PM-2 additionally binds to lymph node metastasis arising from stomach adenocarcinoma (intestinal and diffuse), arising from pancreas adenocarcinoma and arising from head and neck squamous cell carcinoma. PM-2 moreover binds to malignant melanoma (metastasis) of rectum, esophagus, parotid gland, skin, nose, colon and adrenal gland.

PM-2 antigen is therefore a good target and PM-2 antibodies and functional fragments thereof a good therapy for reducing or inhibiting establishment or formation of metastatic tumors, or growth of established metastatic tumors, arising from these and other cancers, and reducing the risk of cancer relapse or progression to metastatic tumor formation or establishment, or growth or proliferation of established or formed metastasis.

1.-3. (canceled)

4. A method for reducing or inhibiting formation or establishment of metastases arising from a neoplasia, tumor or cancer in a subject in need of treatment, comprising administering to the subject an amount of an antibody or functional fragment thereof that competes with PM-2 antibody, produced by a cell line DSMZ Deposit No. DSM ACC 2600, or represented by heavy and light chain sequences set forth as SEQ ID NOs:1 and 2, for binding to HT-29, CACO-2, COLO-320, COLO-206F, ASPC-1, BXPC-3 or A549 cells, effective to reduce or inhibit formation or establishment of metastases arising from a neoplasia, tumor or cancer in the subject.

5. A method for reducing or inhibiting growth, proliferation, mobility or invasiveness of neoplastic, tumor or cancer cells that can develop into or give rise to a metastasis in a subject in need of treatment, comprising administering to the subject an amount of an antibody or functional fragment thereof that competes with PM-2 antibody, produced by a cell line DSMZ Deposit No. DSM ACC 2600, or represented by heavy and light chain sequences set forth as SEQ ID NOs:1 and 2, for binding to HT-29, CACO-2, COLO-320, COLO-206F, ASPC-1, BXPC-3 or A549 cells, effective to reduce or inhibit growth, proliferation, mobility or invasiveness of neoplastic, tumor or cancer cells that can develop into or give rise to the metastasis.

6. A method for reducing or inhibiting neoplasia, tumor or cancer relapse, or neoplasia, tumor or cancer progression in a subject in need of treatment, comprising administering to the subject an amount of an antibody or functional fragment thereof that competes with PM-2 antibody, produced by a cell line DSMZ Deposit No. DSM ACC 2600, or represented by heavy and light chain sequences set forth as SEQ ID NOs:1 and 2, for binding to HT-29, CACO-2, COLO-320, COLO-206F, ASPC-1, BXPC-3 or A549 cells, effective to reduce or inhibit neoplasia, tumor or cancer relapse, or neoplasia, tumor or cancer progression in the subject.

7.-8. (canceled)

9. The method of claim 5, wherein the neoplasia, tumor, cancer, or metastasis affects or is present at least in part in brain, spine, head or neck, breast, esophagus, mouth, nasopharynx, nose or sinuses, thyroid, head or neck, gastrointestinal tract, stomach, small intestine, duodenum, ileum, jejunum, lung, liver, pancreas, kidney, adrenal gland, bladder, colon, rectum, genito-urinary tract, prostate, uterus, endometrium, cervix, ovary, bone marrow, lymph, blood, bone, testes, penis, skin or muscle, or hematopoetic system.

10. The method of claim 5, wherein the neoplasia, tumor, cancer, or metastasis is haematopoetic.

11. The method of claim 5, wherein the neoplasia, tumor, cancer, or metastasis comprises a sarcoma, carcinoma, adenocarcinoma, melanoma, myeloma, blastoma, glioma, lymphoma or leukemia.

12. The method of claim 5, wherein neoplasia, tumor, cancer, or metastasis comprises a lung adenocarcinoma, lung carcinoma, diffuse or interstitial gastric carcinoma, colon adenocarcinoma, prostate adenocarcinoma, esophagus carcinoma, breast carcinoma, pancreas adenocarcinoma, ovarian adenocarcinoma, adenocarcinoma of the adrenal gland, adenocarcinoma of the endometrium or uterine adenocarcinoma.

13. The method of claim 5, wherein the neoplasia, tumor, cancer, or metastasis comprises a stage I, II, III, IV or V neoplasia, tumor cancer, or metastasis.

14.-15. (canceled)

16. The method of claim 5, wherein the neoplasia, tumor, cancer, or metastasis is solid or liquid.

17. The method of claim 5, wherein the PM-2 antibody or functional fragment is administered to the subject locally, regionally, or systemically.

18. (canceled)

19. The method of claim 5, wherein the treatment reduces or decreases metastasis numbers, volume or size, inhibits or prevents an increase in metastasis numbers, volume or size, inhibits progression or worsening of the neoplasia, tumor, cancer, or metastasis, stimulates metastasis cell lysis or apoptosis, or inhibits, reduces or decreases metastasis growth, proliferation or survival.

20.-21. (canceled)

22. The method of claim 5, further comprising administering to the subject an anti-cell proliferative or immune-enhancing treatment or therapy.

23.-29. (canceled)

30. The method of claim 5, wherein the subject is a mammal.

31. The method of claim 30, wherein the subject is a human.

32. (canceled)

33. A method for treating metastasis of a neoplasia, tumor or cancer in a subject in need of treatment, comprising administering to the subject an amount of an antibody or functional fragment comprising a heavy and a light chain sequence at least 70% identical to a heavy or light chain sequence set forth as SEQ ID NOs:1 or 2, effective to treat metastasis of the neoplasia, tumor or cancer in the subject.

34.-64. (canceled)

65. The method of claim 33, wherein the antibody or functional fragment thereof comprises a heavy and a light chain sequence at least 70% identical to a heavy and a light chain sequence set forth as SEQ ID NOs:1 and 2.

66. The method of claim 33, wherein the antibody or functional fragment thereof comprises a heavy and a light chain sequence with one or more CDRs at least 80% identical to one or more CDRs of the heavy and light chain sequences set forth as SEQ ID NOs:1 and 2.

67. The method of claim 33, wherein the antibody or functional fragment thereof comprises a heavy and a light chain sequence with one or more CDRs at least 90% identical to one or more CDRs of the heavy and light chain sequences set forth as SEQ ID NOs:1 and 2.

68. The method of claim 33, wherein the antibody or functional fragment thereof comprises a heavy and a light chain sequence with one or more CDRs at least 100% identical to one or more CDRs of the heavy and light chain sequences set forth as SEQ ID NOs:1 and 2.

69. The method of any of claims 5 or 33, wherein the antibody or functional fragment thereof binds to an epitope or an antigen to which PM-2 antibody, produced by a cell line DSMZ Deposit No. DSM ACC 2600, or represented by heavy and light chain sequences set forth as SEQ ID NOs:1 and 2 binds.

70. The method of claim 69, wherein the antigen comprises a protein antigen of about 115 kDa expressed on BXPC-3, MKN or CRL cells.

71. The method of claim 69, wherein the antibody or functional fragment thereof competes with PM-2 antibody, produced by a cell line DSMZ Deposit No. DSM ACC 2600, or represented by heavy and light chain sequences set forth as SEQ ID NOs:1 and 2, for binding to HT-29, CACO-2, COLO-320, COLO-206F, ASPC-1, BXPC-3 or A549 cells.

72. The method of any claim 69, wherein the antibody or functional fragment thereof binds to an epitope to which PM-2 antibody, produced by a cell line DSMZ Deposit No. DSM ACC 2600, or represented by heavy and light chain sequences set forth as SEQ ID NOs:1 and 2 binds, wherein the epitope is present on a protein antigen of about 115 kDa expressed on BXPC-3, MKN or CRL cells.

73. (canceled)
